SEQUENCE LISTING (7 sequences)

SEQ ID NO: 1 - protein, 808 aa, Artificial sequence
  SuSy_At, variant PM1-54-2-E05 (engineered sucrose synthase; source of gene Arabidopsis thaliana)
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLDFLRLHSHQGKNLMLSEKIQNLNTLQHTLRKAEEYLAELKSETLYEEFEAKFEEIGLERGWGDNAERVLDMIRLLLDLLEAPDPSTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENDEHLCVLKDKKKPILFTMARLDRVKNLSGLVEWYGKNTRLRELVNLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADLLADFFTKCKEDPSHWDEISKGGLQRIEEKYTWQIYSQRLLTLTGVYGFWKHVSNLDRLEHRRYLEMFYALKYRPLAQAVPLAQDD

SEQ ID NO: 2 - protein, 460 aa, Stevia rebaudiana
  UGT74G1 (glucosyltransferase)
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 3 - protein, 481 aa, Stevia rebaudiana
  UGT85C2 (glucosyltransferase)
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 4 - protein, 442 aa, Artificial sequence
  UGTSl2 variant 0234 (engineered glucosyltransferase; source of gene Solanum lycopersicum)
MATNLRVLMFPWLAYGHISPFLNIAKQLADRGFLIYLCSTRINLESIIKKIPEKYADSIHLIELQLPELPELPPHYHTTNGLPPHLNPTLHKALKMSKPNFSRILQNLKPDLLIYDVLQPWAEHVANEQGIPAGKLLVSCAAVFSYFFSFRKNPGVEFPFPAIHLPEVEKVKIREILAKEPEEGGRLDEGNKQMMLMCTSRTIEAKYIDYCTELCNWKVVPVGPPFQDLITNDADNKELIDWLGTKPENSTVFVSFGSEYFLSKEDMEEIAFALEASNVNFIWVVRFPKGEERNLEDALPEGFLERIGERGRVLDKFAPQPRILNHPSTGGFISHCGWNSVMESIDFGVPIIAMPIHNDQPINAKLMVELGVAVEIVRDDDGKIHRGEIAEALKSVVTGETGEILRAKVREISKNLKSIRDEEMDAVAEELIQLCRNSNKSK

SEQ ID NO: 5 - protein, 458 aa, Artificial sequence
  UGT76G1 variant 0042 (engineered glucosyltransferase; source of gene Stevia rebaudiana)
MENKTETTVRRRRRIILFPVPFQGHINPILQLANVLYSKGFAITILHTNFNKPKTSNYPHFTFRFILDNDPQDERISNLPTHGPLAGMRIPIINEHGADELRRELELLMLASEEDEEVSCLITDALWYFAQDVADSLNLRRLVLMTSSLFNFHAHVSLPQFDELGYLDPDDKTRLEEQASGFPMLKVKDIKSAYSNWQIGKEILGKMIKQTKASSGVIWNSFKELEESELETVIREIPAPSFLIPLPKHLTASSSSLLDHDRTVFEWLDQQAPSSVLYVSFGSTSEVDEKDFLEIARGLVDSGQSFLWVVRPGFVKGSTWVEPLPDGFLGERGKIVKWVPQQEVLAHPAIGAFWTHSGWNSTLESVCEGVPMIFSSFGGDQPLNARYMSDVLRVGVYLENGWERGEVVNAIRRVMVDEEGEYIRQNARVLKQKADVSLMKGGSSYESLESLVSYISSL

SEQ ID NO: 6 - protein, 462 aa, Oryza sativa Japonica
  EUGT11 (glucosyltransferase)
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 7 - protein, 473 aa, Stevia rebaudiana
  UGT91D2 (glucosyltransferase)
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEVPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNTRAVAIDHES

(19) United States
(12) Patent Application Publication
(10) Pub. No.: US 2024/0360489 A1
(43) Pub. Date: Oct. 31, 2024

(54) HIGH-PURITY STEVIOL GLYCOSIDES

(71) Applicant: PURECIRCLE SDN BHD, Negeri Sembilan (MY)

(72) Inventors: Avetik MARKOSYAN, Negeri Sembilan (MY); Siew Yin CHOW, Negeri Sembilan (MY); Kristina CHKHAN, Negeri Sembilan (MY); Khairul NIZAM BIN NAWI, Negeri Sembilan (MY); Saravanan A/L RAMANDACH, Negeri Sembilan (MY); Mohamad Afzaal Bin HASIM, Negeri Sembilan (MY); Pei Chen KOH, Negeri Sembilan (MY)

(21) Appl. No.: 18/571,856

(22) PCT Filed: Jun. 28, 2022
(86) PCT No.: PCT/MY2022/050056

§ 371 Date: Dec. 19, 2023

(30) Foreign Application Priority Data
Jun. 29, 2021 (MY) PI2021003679

(51) Int. Cl. C12P 19/56; A23L 2/52; A23L 2/56; A23L 2/60; A23L 27/00; A23L 27/30; C07H 15/256; C12N 9/10

(52) U.S. Cl. CPC C12P 19/56; A23L 2/52; A23L 2/56; A23L 2/60; A23L 27/36; A23L 27/88; C07H 15/256; C12N 9/1051

(57) ABSTRACT

Methods of preparing highly purified steviol glycosides are described. The methods include utilizing enzyme preparations and recombinant microorganisms for converting various starting compositions to target steviol glycosides. The highly purified steviol glycosides are useful as non-caloric sweetener, flavor enhancer, sweetness enhancer, flavor stabilizer, flavoring with modifying properties (FMP), foaming suppressor and solubility enhancing agent in consumable products such as any food, beverages, pharmaceutical compositions, tobacco products, nutraceutical compositions, and oral hygiene compositions.

### TECHNICAL FIELD

The present invention relates to a process for preparing compositions comprising steviol glycosides, including highly purified steviol glycoside compositions.

### BACKGROUND OF THE INVENTION

High intensity sweeteners possess a sweetness level that is many times greater than the sweetness level of sucrose. They are essentially non-caloric and are commonly used in diet and reduced-calorie products, including foods and beverages. High intensity sweeteners do not elicit a glycemic response, making them suitable for use in products targeted to diabetics and others interested in controlling for their intake of carbohydrates.

Steviol glycosides are a class of compounds found in the leaves of Stevia rebaudiana Bertoni, a perennial shrub of the Asteraceae (Compositae) family native to certain regions of South America. They are characterized structurally by a single base, steviol, differing by the presence of carbohydrate residues at positions C13 and C19. They accumulate in Stevia leaves, composing approximately 10%-20% of the total dry weight. On a dry weight basis, the four major glycosides found in the leaves of Stevia typically include stevioside (9.1%), rebaudioside A (3.8%), rebaudioside C (0.6-1.0%) and dulcoside A (0.3%). Other known steviol glycosides include rebaudioside B, C, D, E, F and M, steviolbioside and rubusoside.

Although methods are known for preparing steviol glycosides from Stevia rebaudiana, many of these methods are unsuitable for use commercially.

Accordingly, there remains a need for simple, efficient, and economical methods for preparing compositions comprising steviol glycosides, including highly purified steviol glycoside compositions.

### SUMMARY OF THE INVENTION

As used herein, the abbreviation term “reb” refers to “rebaudioside”. Both terms have the same meaning and may be used interchangeably.

As used herein, “biocatalysis” or “biocatalytic” refers to the use of natural or genetically engineered biocatalysts, such as enzymes, or cells comprising one or more enzyme, capable of single or multiple step chemical transformations on organic compounds. Biocatalysis processes include fermentation, biosynthesis, bioconversion and biotransformation processes. Both isolated enzymes, and whole-cell biocatalysis methods are known in the art. Biocatalyst protein enzymes can be naturally occurring or recombinant proteins.

As used herein, the term “steviol glycoside(s)” refers to a glycoside of steviol, including, but not limited to, steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM, rebaudioside M4, naturally occurring steviol glycosides, synthetic steviol glycosides, e.g. enzymatically glucosylated steviol glycosides, and combinations thereof.

The present invention provides a process for preparing a composition comprising a target steviol glycoside by contacting a starting composition comprising an organic substrate with a microbial cell and/or enzyme preparation, thereby producing a composition comprising a target steviol glycoside.

The starting composition can be any organic compound comprising at least one carbon atom. In one embodiment, the starting composition is selected from the group consisting of steviol glycosides, polyols or sugar alcohols, various carbohydrates.

The target steviol glycoside can be any steviol glycoside. In one embodiment, the target steviol glycoside is steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM, rebaudioside M4 or other synthetic steviol glycoside.

In one embodiment, the target steviol glycoside is rebaudioside E.

In one embodiment, the target steviol glycoside is rebaudioside E2.

In one embodiment, the target steviol glycoside is rebaudioside E3.

In one embodiment, the target steviol glycoside is rebaudioside E8.

In one embodiment, the target steviol glycoside is rebaudioside E9.

In one embodiment, the target steviol glycoside is rebaudioside E10.

In one embodiment, the target steviol glycoside is rebaudioside E11.

In one embodiment, the target steviol glycoside is rebaudioside E12.

In one embodiment, the target steviol glycoside is rebaudioside D9.

In one embodiment, the target steviol glycoside is rebaudioside D10.

In one embodiment, the target steviol glycoside is rebaudioside D11.

In one embodiment, the target steviol glycoside is rebaudioside D12.

In one embodiment, the target steviol glycoside is rebaudioside AM.

In one embodiment, the target steviol glycoside is rebaudioside M4.

In some preferred embodiments enzyme preparation comprising one or more enzymes, or a microbial cell comprising one or more enzymes, capable of converting the starting composition to target steviol glycosides are used. The enzyme can be located on the surface and/or inside the cell. The enzyme preparation can be provided in the form of a whole cell suspension, a crude lysate or as purified enzyme(s). The enzyme preparation can be in free form or immobilized to a solid support made from inorganic or organic materials.

In some embodiments, a microbial cell comprises the necessary enzymes and genes encoding thereof for converting the starting composition to target steviol glycosides. Accordingly, the present invention also provides a process for preparing a composition comprising a target steviol glycoside by contacting a starting composition comprising an organic substrate with a microbial cell comprising at least one enzyme capable of converting the starting composition to target steviol glycosides, thereby producing a medium comprising at least one target steviol glycoside.

The enzymes necessary for converting the starting composition to target steviol glycosides include the steviol biosynthesis enzymes, NDP-glucosyltransferases (NGTs), ADP-glucosyltransferases (AGTs), CDP-glucosyltransferases (CGTs), GDP-glucosyltransferases (GGTs), TDP-glucosyltransferases (TDPs), UDP-glucosyltransferases (UGTs) and/or NDP-recycling enzyme, ADP-recycling enzyme, CDP-recycling enzyme, GDP-recycling enzyme, TDP-recycling enzyme, and/or UDP-recycling enzyme.

In one embodiment, the steviol biosynthesis enzymes include mevalonate (MVA) pathway enzymes.

In another embodiment, the steviol biosynthesis enzymes include non-mevalonate 2-C-methyl-D-erythritol-4-phosphate pathway (MEP/DOXP) enzymes.

In one embodiment the steviol biosynthesis enzymes are selected from the group including geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase etc.

The UDP-glucosyltransferase can be any UDP-glucosyltransferase capable of adding at least one glucose unit to steviol and/or a steviol glycoside substrate to provide the target steviol glycoside.

As used hereinafter, the term “SuSy_AT”, unless specified otherwise, refers to sucrose synthase having amino-acid sequence “SEQ ID 1” or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 1 polypeptide as well as isolated nucleic acid molecules that code for those polypeptides. Sucrose synthase produces UDP-glucose by transferring glucose from a glucose donor, e.g. sucrose to UDP. UDP-glucose is then used by glucosyltransferase for transferring the glucose to a steviol-containing compound to produce a target compound.

As used hereinafter, the term “UGT74G1”, unless specified otherwise, refers to UDP-glucosyltransferase having amino-acid sequence SEQ ID 2 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 2 polypeptide as well as isolated nucleic acid molecules that code for those polypeptides. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to the SEQ ID 2 polypeptide.

As used hereinafter, the term “UGT85C2”, unless specified otherwise, refers to UDP-glucosyltransferase having amino-acid sequence SEQ ID 3 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 3 polypeptide as well as isolated nucleic acid molecules that code for those polypeptides. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to the SEQ ID 3 polypeptide.

As used hereinafter, the term “UGTS12”, unless specified otherwise, refers to UDP-glucosyltransferase having amino-acid sequence SEQ ID 4 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 4 polypeptide as well as isolated nucleic acid molecules that code for those polypeptides. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to the SEQ ID 4 polypeptide.

As used hereinafter, the term “UGT76G1”, unless specified otherwise, refers to UDP-glucosyltransferase having amino-acid sequence SEQ ID 5 or a polypeptide having substantial (>85%, >86%, >87%, >88%, >89%, >90%, >91%, >92%, >93%, >94%, >95%, >96%, >97%, >98%, >99%) amino-acid sequence identity to the SEQ ID 5 polypeptide as well as isolated nucleic acid molecules that code for those polypeptides. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to the SEQ ID 5 polypeptide.

In one embodiment, steviol biosynthesis enzymes and UDP-glucosyltransferases are produced in a microbial cell. The microbial cell may be, for example, E. coli, Saccharomyces sp., Aspergillus sp., Pichia sp., Bacillus sp., Yarrowia sp. etc. In another embodiment, the UDP-glucosyltransferases are synthesized.

In one embodiment, the UDP-glucosyltransferase is selected from group including UGT74G1, UGT85C2, UGTS12, UGT76G1, EUGT11, UGT91D2 and UGTs having substantial (>85%, >86%, >87%, >88%, >89%, >90%, >91%, >92%, >93%, >94%, >95%, >96%, >97%, >98%, >99%) amino-acid sequence identity to SEQ ID 2, SEQ ID 3, SEQ ID 4, SEQ ID 5, SEQ ID 6 and SEQ ID 7, respectively as well as isolated nucleic acid molecules that code for these UGTs. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to these polypeptides.

In one embodiment, steviol biosynthesis enzymes, UGTs, and UDP-glucose recycling system are present in one microorganism (microbial cell). The microorganism may be for example, E. coli, Saccharomyces sp., Aspergillus sp., Pichia sp., Bacillus sp., Yarrowia sp.

In one embodiment, the UDP-glucosyltransferase is any UDP-glucosyltransferase capable of adding at least one glucose unit to steviol or any starting steviol glycoside bearing an —OH functional group at C13 to give a target steviol glycoside having an —O-glucose beta glucopyranoside glycosidic linkage at C13. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to steviol or any starting steviol glycoside bearing a —COOH functional group at C19 to give a target steviol glycoside having a —COO-glucose beta-glucopyranoside glycosidic linkage at C19. In a particular embodiment, the UDP-glucosyltransferase is UGT74G1, or a UGT having >85% amino-acid sequence identity with SEQ ID 2.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1->2 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1->3 glucopyranoside glycosidic linkage(s) at the newly formed bond glycosidic bond(s). In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1-4 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1->6 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1->2 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1->3 glucopyranoside glycosidic linkage(s) at the newly formed bond glycosidic bond(s). In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1-4 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1->6 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In one embodiment, the UDP-glucosyltransferase is any UDP-glucosyltransferase capable of adding at least one glucose unit to steviol to form steviolmonoside. In a particular embodiment, the UDP-glucosyltransferase is UGT85C2 or a UGT having >85% amino-acid sequence identity with UGT85C2 or a UGT having >85% amino-acid sequence identity with SEQ ID 3.

In another embodiment, the UDP-glucosyltransferase is any UDP-glucosyltransferase capable of adding at least one glucose unit to steviol to form steviolmonoside A. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to steviolmonoside to form steviolbioside. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to steviolmonoside to form rubusoside. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to steviolmonoside to form steviolbioside F. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to steviolmonoside A to form rubusoside. In a particular embodiment, the UDP-glucosyltransferase is UGT85C2 or a UGT having >85% amino-acid sequence identity with UGT85C2 or a UGT having >85% amino-acid sequence identity with SEQ ID 3.

In another embodiment, the UDP-glucosyltransferase is any UDP-glucosyltransferase capable of adding at least one glucose unit to steviolmonoside A to form steviolbioside A. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to steviolmonoside A to form steviolbioside B. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to steviolmonoside A to form steviolbioside G. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to steviolbioside to form stevioside. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to steviolbioside to form stevioside L In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rubusoside to form stevioside. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rubusoside to form stevioside A. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rubusoside to form stevioside B. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rubusoside to form stevioside J. In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rubusoside to form stevioside L. In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to steviolbioside A to form stevioside A. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to steviolbioside A to form stevioside C. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to steviolbioside A to form stevioside K. In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to steviolbioside B to form stevioside B. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to steviolbioside B to form stevioside C. In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to steviolbioside G to form stevioside K. In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to steviolbioside G to form stevioside L. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to stevioside to form rebaudioside E. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside to form rebaudioside E2. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside to form rebaudioside E8. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside to form rebaudioside E9. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside A to form rebaudioside E. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside A to form rebaudioside E3. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside A to form rebaudioside E11. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside A to form rebaudioside E12. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside B to form rebaudioside E2. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside B to form rebaudioside E3. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside B to form rebaudioside E10. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside C to form rebaudioside E3. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to stevioside I to form rebaudioside E9. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to stevioside J to form rebaudioside E9. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside J to form rebaudioside E10. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside J to form rebaudioside E11. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside K to form rebaudioside E12. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to stevioside L to form rebaudioside E8. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside L to form rebaudioside E12. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E to form rebaudioside D9. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E to form rebaudioside D10. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside E to form rebaudioside AM. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside E2 to form rebaudioside D11. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside E2 to form rebaudioside AM. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E3 to form rebaudioside D12. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside E3 to form rebaudioside AM. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E8 to form rebaudioside D9. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E9 to form rebaudioside D10. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E9 to form rebaudioside D11. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside E10 to form rebaudioside D11. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E10 to form rebaudioside D12. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E11 to form rebaudioside D10. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E11 to form rebaudioside D12. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside E12 to form rebaudioside D9. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside D10 to form rebaudioside M4. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside D11 to form rebaudioside M4. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside D12 to form rebaudioside M4. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside AM to form rebaudioside M4. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside to form rebaudioside D9. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside to form rebaudioside M4. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

Optionally, the method of the present invention further comprises recycling UDP to provide UDP-glucose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-glucosyltransferase and UDP-glucose. The UDP recycling enzyme can be sucrose synthase SuSy_At or a sucrose synthase having >85% amino-acid sequence identity with SuSy_At and the recycling substrate can be sucrose.

In one embodiment, the recycling catalyst is sucrose synthase SuSy_At or a sucrose synthase having >85% amino-acid sequence identity with SuSy_At.

In one embodiment, the recycling substrate for UDP-glucose recycling catalyst is sucrose.

Optionally, the method of the present invention further comprises the use of transglycosidases that use oligo- or poly-saccharides as the sugar donor to modify recipient target steviol glycoside molecules. Non-limiting examples include cyclodextrin glycosyltransferase (CGTase), fructofuranosidase, amylase, saccharase, glucosucrase, beta-h-fructosidase, beta-fructosidase, sucrase, fructosylinvertase, alkaline invertase, acid invertase, fructofuranosidase. In some embodiments, glucose and sugar(s) other than glucose, including but not limited to fructose, xylose, rhamnose, arabinose, deoxyglucose, galactose are transferred to the recipient target steviol glycosides. In one embodiment, the recipient steviol glycoside is stevioside, rebaudioside E, rebaudioside E2, rebaudioside E8, rebaudioside E9, rebaudioside D9, rebaudioside D10, rebaudioside D11, rebaudioside AM and/or rebaudioside M4.

Optionally, the method of the present invention further comprises separating the target steviol glycoside from the medium to provide a highly purified target steviol glycoside composition. The target steviol glycoside can be separated by at least one suitable method, such as, for example, crystallization, separation by membranes, centrifugation, extraction, chromatographic separation or a combination of such methods.

In one embodiment, the target steviol glycoside can be produced by the enzyme. In another embodiment, the target steviol glycoside is produced by enzymatic conversion. In one another embodiment, the converted steviol glycoside can be continuously removed from the medium. In yet another embodiment, the target steviol glycoside is separated after the completion of the conversion reaction.

In one embodiment, the target steviol glycoside can be produced within the microorganism. In another embodiment, the target steviol glycoside can be secreted out in the medium. In one another embodiment, the released steviol glycoside can be continuously removed from the medium. In yet another embodiment, the target steviol glycoside is separated after the completion of the conversion reaction.

In one embodiment, separation produces a composition comprising greater than about 80% by weight of the target steviol glycoside on an anhydrous basis, i.e., a highly purified steviol glycoside composition. In another embodiment, separation produces a composition comprising greater than about 90% by weight of the target steviol glycoside. In particular embodiments, the composition comprises greater than about 95% by weight of the target steviol glycoside. In other embodiments, the composition comprises greater than about 99% by weight of the target steviol glycoside.

Unless otherwise indicated, weight percentages presented herein (e.g. percent by weight) are by weight of the total composition.

The target steviol glycoside can be in any polymorphic or amorphous form, including hydrates, solvates, anhydrous or combinations thereof.

Purified target steviol glycosides can be used in consumable products as a sweetener, flavor stabilizer, flavoring with modifying properties (FMP), foaming suppressor and/or solubility enhancing agent. Suitable consumer products include, but are not limited to, food, beverages, pharmaceutical compositions, tobacco products, nutraceutical compositions, oral hygiene compositions, and cosmetic compositions.

### BRIEF DESCRIPTION OF THE DRAWINGS

FIG. 1a shows the chemical structure of Steviolbioside F.

FIG. 1b shows the chemical structure of Steviolbioside G.

FIG. 1c shows the chemical structure of Stevioside I.

FIG. 1d shows the chemical structure of Stevioside J.

FIG. 1e shows the chemical structure of Stevioside K.

FIG. 1f shows the chemical structure of Stevioside L.

FIG. 1g shows the chemical structure of Rebaudioside E2.

FIG. 1h shows the chemical structure of Rebaudioside E8.

FIG. 1i shows the chemical structure of Rebaudioside E9.

FIG. 1j shows the chemical structure of Rebaudioside E10.

FIG. 1k shows the chemical structure of Rebaudioside E11.

FIG. 1l shows the chemical structure of Rebaudioside E12.

FIG. 1m shows the chemical structure of Rebaudioside D9.

FIG. 1n shows the chemical structure of Rebaudioside D10.

FIG. 1o shows the chemical structure of Rebaudioside D11.

FIG. 1p shows the chemical structure of Rebaudioside D12.

FIG. 1q shows the chemical structure of Rebaudioside M4.

FIG. 2a-2d show the pathways of producing rebaudioside D9 and various steviol glycosides from steviol.

FIG. 3a-3e show the pathways of producing rebaudioside M4 and various steviol glycosides from steviol.

FIG. 4a shows the biocatalytic production of rebaudioside E from stevioside using the enzymes UGTS12 and UGT76G1 and concomitant recycling of UDP to UDP-glucose via sucrose synthase SuSy_At.

FIG. 4b shows the biocatalytic production of rebaudioside E2 from stevioside using the enzymes UGTS12 and UGT76G1 and concomitant recycling of UDP to UDP-glucose via sucrose synthase SuSy_At.

FIG. 4c shows the biocatalytic production of rebaudioside E8 from stevioside using the enzymes UGTS12 and UGT76G1 and concomitant recycling of UDP to UDP-glucose via sucrose synthase SuSy_At.

FIG. 4d shows the biocatalytic production of rebaudioside E9 from stevioside using the enzymes UGTS12 and UGT76G1 and concomitant recycling of UDP to UDP-glucose via sucrose synthase SuSy_At.

FIG. 4e shows the biocatalytic production of rebaudioside D9 from stevioside using the enzymes UGTS12 and UGT76G1 and concomitant recycling of UDP to UDP-glucose via sucrose synthase SuSy_At.

FIG. 4f shows the biocatalytic production of rebaudioside D10 from stevioside using the enzymes UGTS12 and UGT76G1 and concomitant recycling of UDP to UDP-glucose via sucrose synthase SuSy_At.

FIG. 4g shows the biocatalytic production of rebaudioside D11 from stevioside using the enzymes UGTS12 and UGT76G1 and concomitant recycling of UDP to UDP-glucose via sucrose synthase SuSy_At.

FIG. 4h shows the biocatalytic production of rebaudioside AM from stevioside using the enzymes UGTS12 and UGT76G1 and concomitant recycling of UDP to UDP-glucose via sucrose synthase SuSy_At.

FIG. 4i shows the biocatalytic production of rebaudioside M4 from stevioside using the enzymes UGTS12 and UGT76G1 and concomitant recycling of UDP to UDP-glucose via sucrose synthase SuSy_At.

FIG. 5a shows the biocatalytic production of rebaudioside D9 from rebaudioside E using the enzymes UGTS12 and UGT76G1 and concomitant recycling of UDP to UDP-glucose via sucrose synthase SuSy_At.

FIG. 5b shows the biocatalytic production of rebaudioside D9 from rebaudioside E8 using the enzymes UGTS12 and UGT76G1 and concomitant recycling of UDP to UDP-glucose via sucrose synthase SuSy_At.

FIG. 5c shows the biocatalytic production of rebaudioside D10 from rebaudioside E using the enzymes UGTS12 and UGT76G1 and concomitant recycling of UDP to UDP-glucose via sucrose synthase SuSy_At.

FIG. 5d shows the biocatalytic production of rebaudioside D10 from rebaudioside E9 using the enzymes UGTS12 and UGT76G1 and concomitant recycling of UDP to UDP-glucose via sucrose synthase SuSy_At.

FIG. 5e shows the biocatalytic production of rebaudioside D11 from rebaudioside E2 using the enzymes UGTS12 and UGT76G1 and concomitant recycling of UDP to UDP-glucose via sucrose synthase SuSy_At.

FIG. 5f shows the biocatalytic production of rebaudioside D11 from rebaudioside E9 using the enzymes UGTS12 and UGT76G1 and concomitant recycling of UDP to UDP-glucose via sucrose synthase SuSy_At.

FIG. 5g shows the biocatalytic production of rebaudioside AM from rebaudioside E using the enzymes UGTS12 and UGT76G1 and concomitant recycling of UDP to UDP-glucose via sucrose synthase SuSy_At.

FIG. 5h shows the biocatalytic production of rebaudioside AM from rebaudioside E2 using the enzymes UGTS12 and UGT76G1 and concomitant recycling of UDP to UDP-glucose via sucrose synthase SuSy_At.

FIG. 5i shows the biocatalytic production of rebaudioside M4 from rebaudioside D10 using the enzymes UGTS12 and UGT76G1 and concomitant recycling of UDP to UDP-glucose via sucrose synthase SuSy_At.

FIG. 5j shows the biocatalytic production of rebaudioside M4 from rebaudioside D11 using the enzymes UGTS12 and UGT76G1 and concomitant recycling of UDP to UDP-glucose via sucrose synthase SuSy_At.

FIG. 5k shows the biocatalytic production of rebaudioside M4 from rebaudioside AM using the enzymes UGTS12 and UGT76G1 and concomitant recycling of UDP to UDP-glucose via sucrose synthase SuSy_At.

FIG. 6a shows the HPLC chromatogram of stevioside. The peak with retention time of 20.958 minutes corresponds to stevioside. The peak with retention time 20.725 minutes corresponds to rebaudioside A. The peak at 32.925 minutes corresponds to rebaudioside B. The peak at 33.930 minutes corresponds to steviolbioside.

FIG. 6b shows the HPLC chromatogram of the product of the biocatalytic production of rebaudioside D9 and rebaudioside M4 molecules from stevioside. The peak at 5.896 minutes corresponds to rebaudioside D9. The peak at 8.775 minutes corresponds to rebaudioside M4. The peak at 9.825 minutes corresponds to rebaudioside AM. The peak at 13.845 minutes corresponds to rebaudioside M. The peak at 32.974 minutes corresponds to rebaudioside B. The peak at 33.979 minutes corresponds to steviolbioside.

FIG. 6c shows the LCMS DAD chromatogram of rebaudioside D9 after purification by HPLC. The peak with retention time of 7.115 minutes corresponds to rebaudioside D9.

FIG. 6d shows the LCMS DAD chromatogram of rebaudioside M4 after purification by HPLC. The peak with retention time of 9.081 minutes corresponds to rebaudioside M4.

FIG. 7a shows the MSD chromatogram of rebaudioside D9.

FIG. 7b shows the mass spectrum of rebaudioside D9.

FIG. 8a shows the MSD chromatogram of rebaudioside M4.

FIG. 8b shows the mass spectrum of rebaudioside M4.

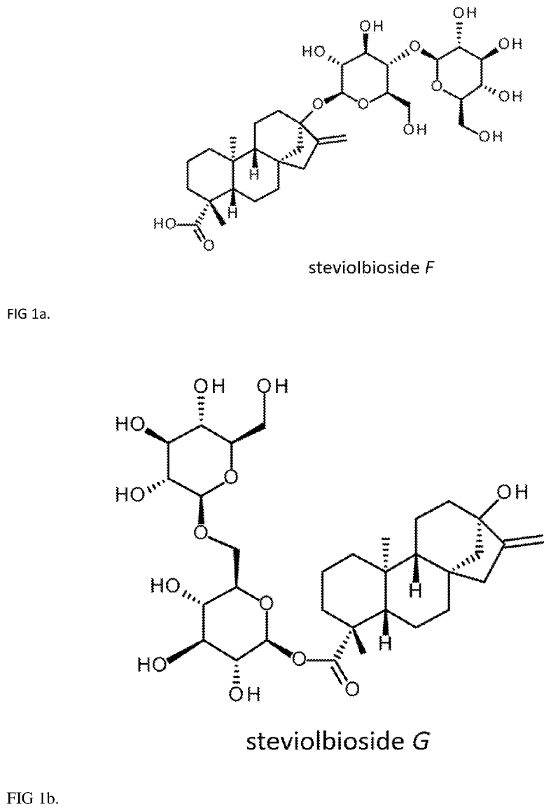

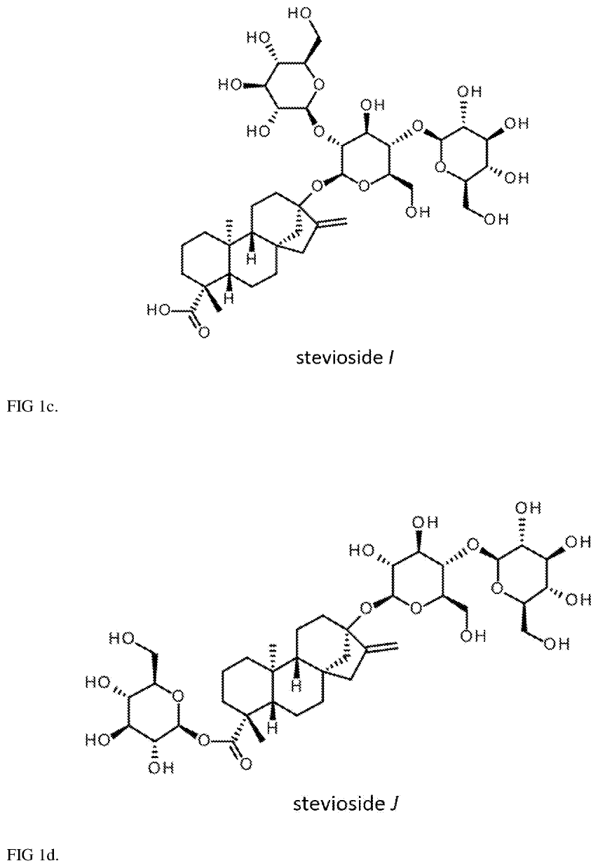

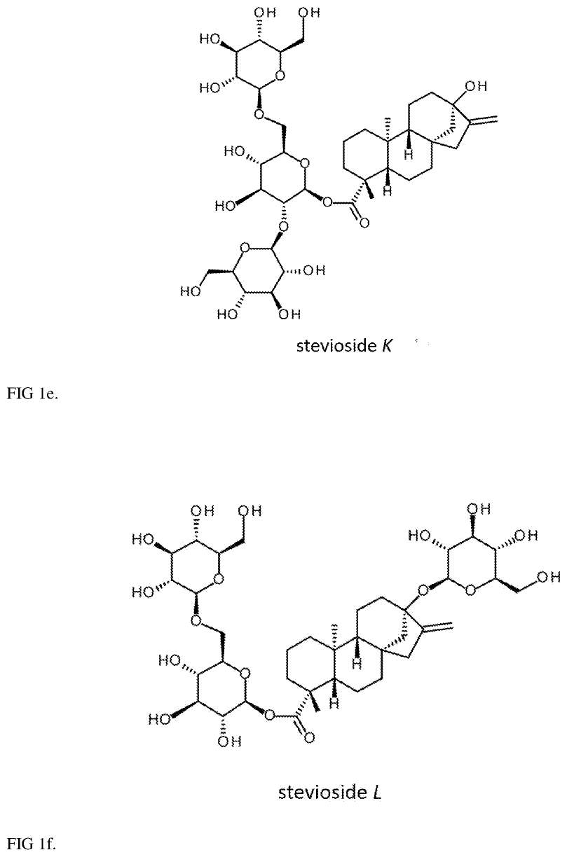

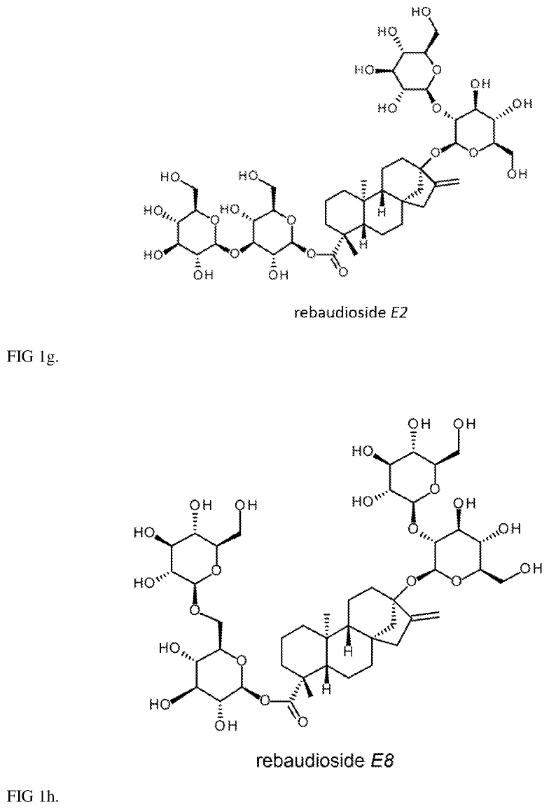

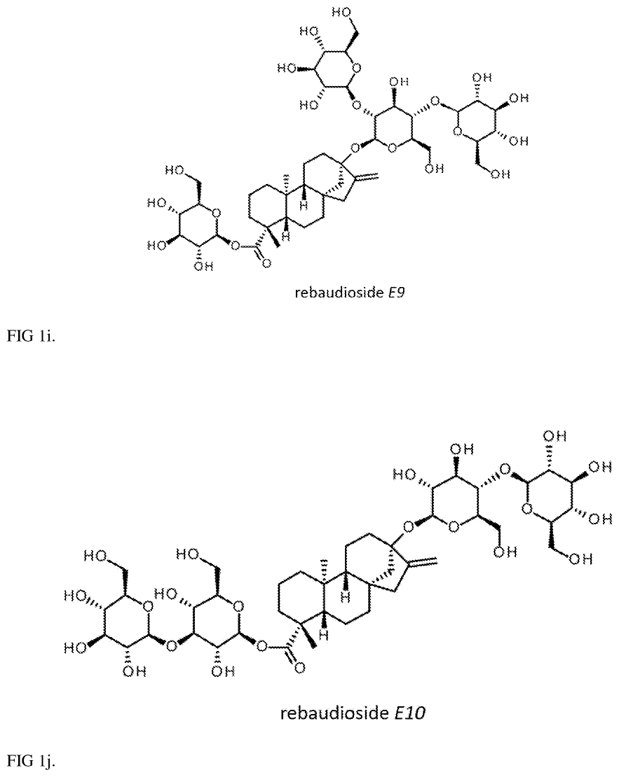

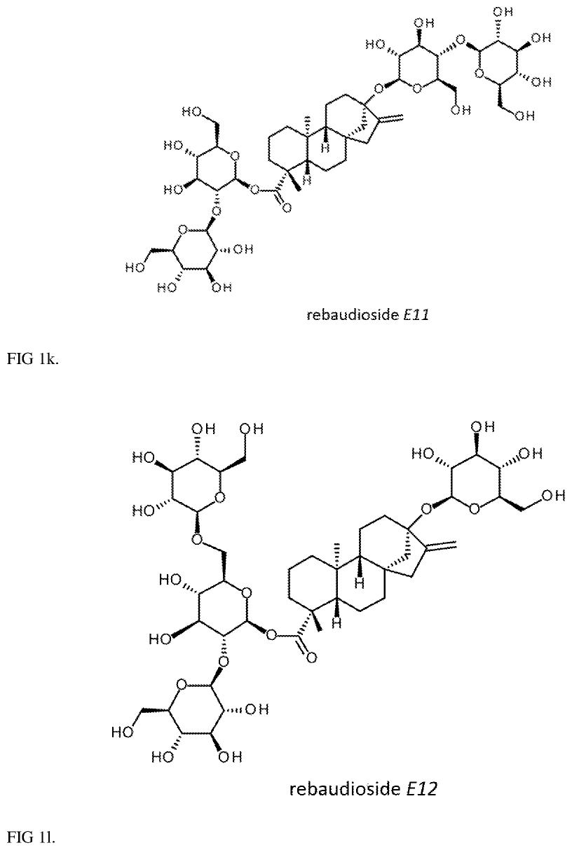

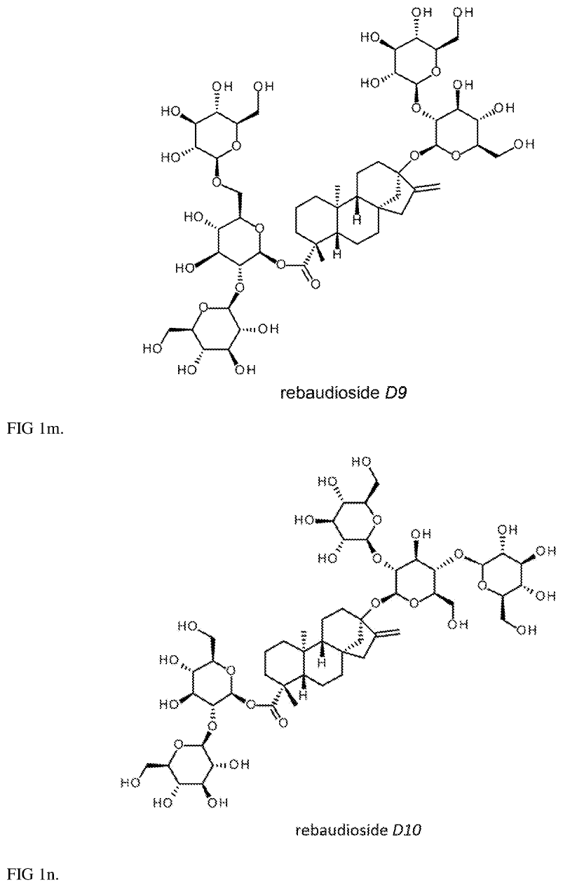

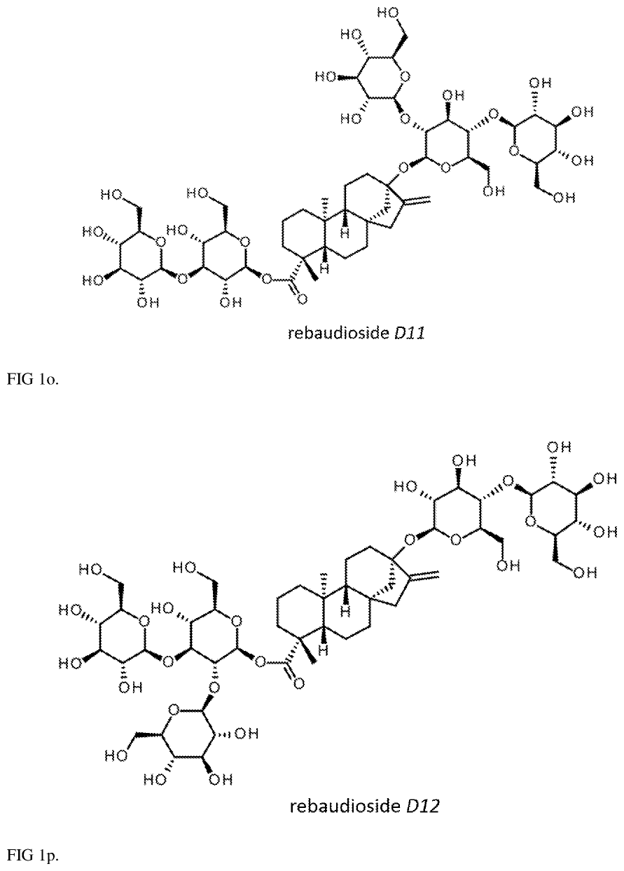

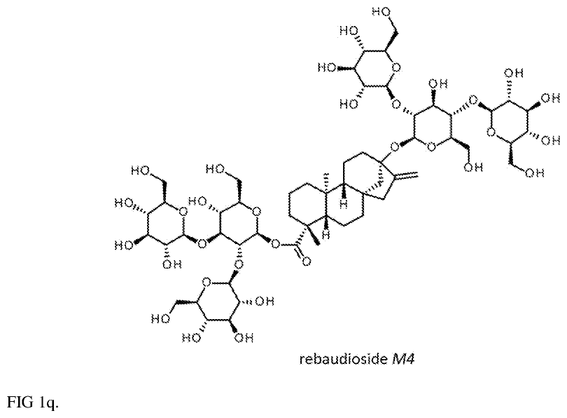

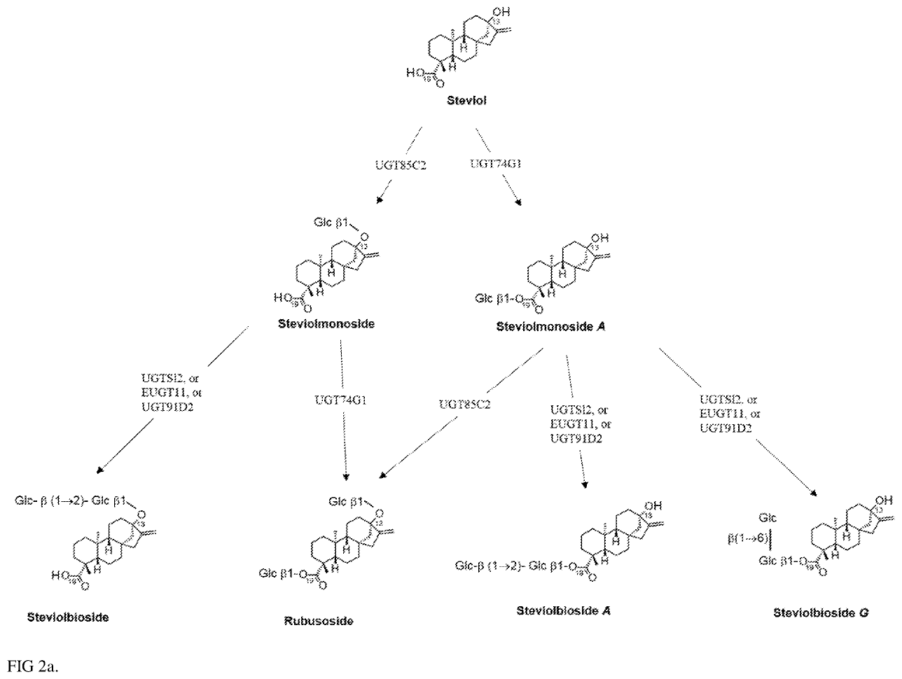

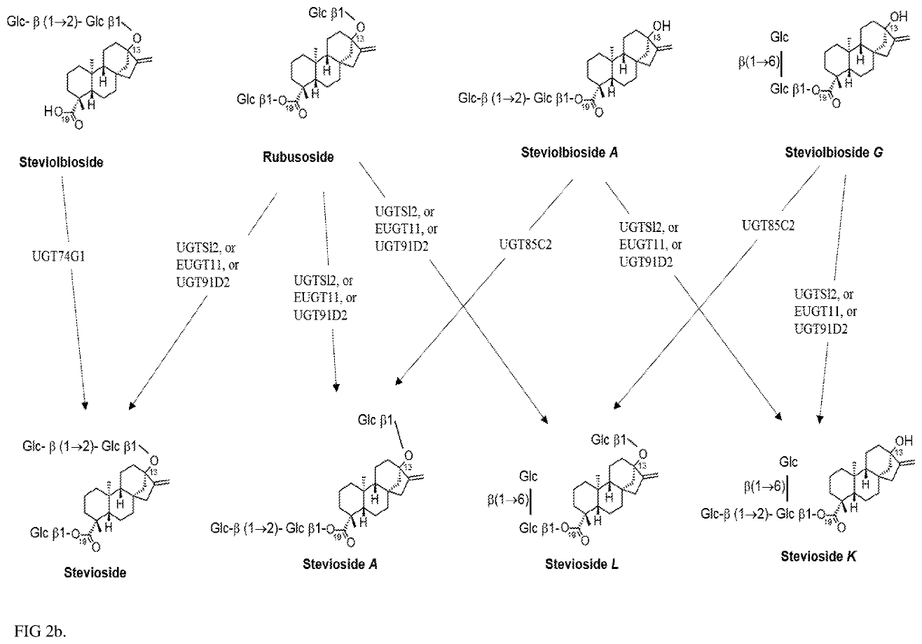

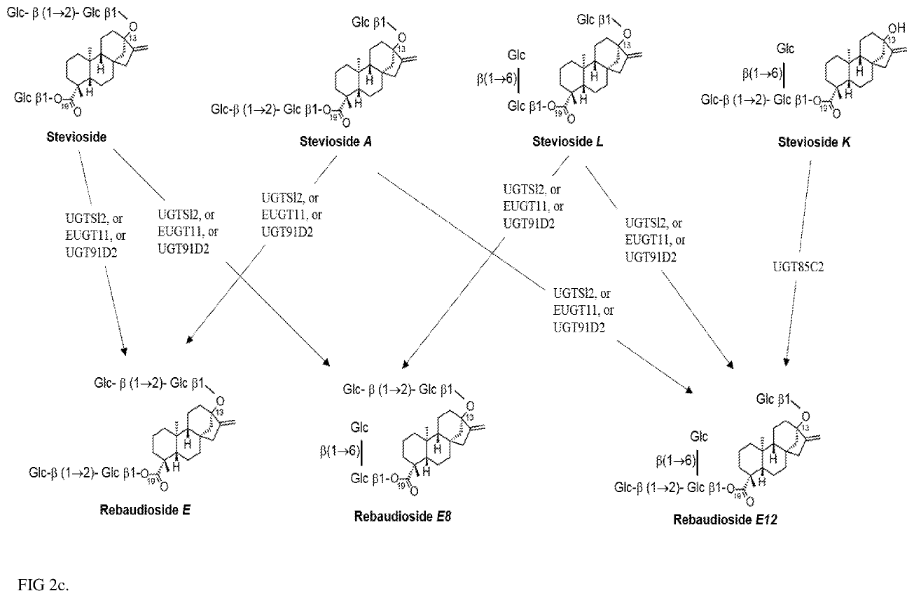

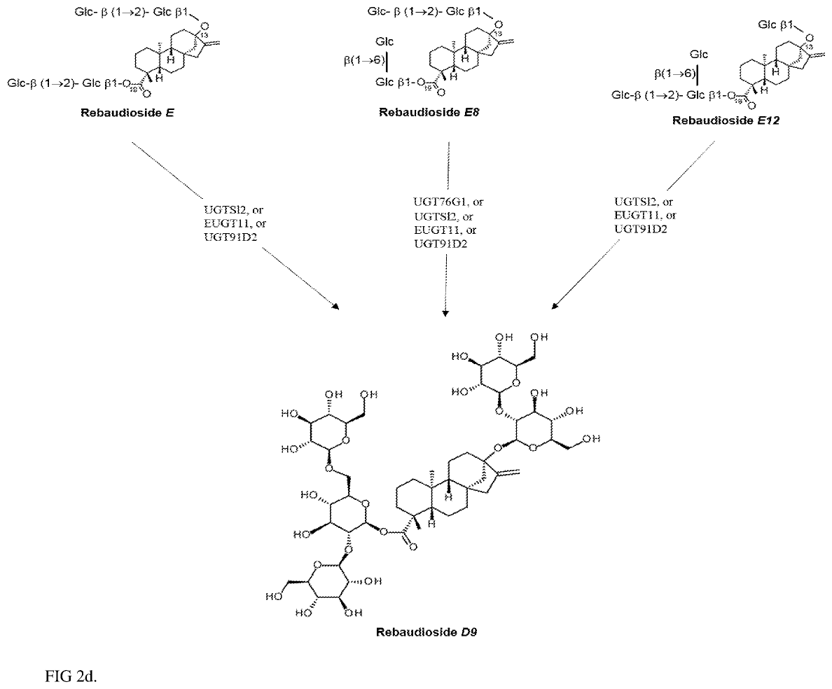

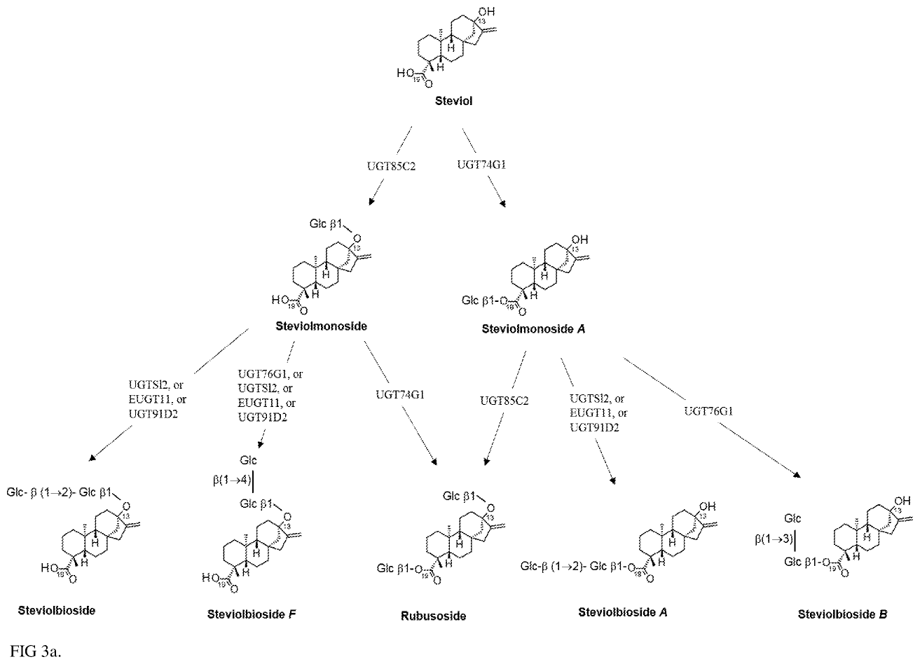

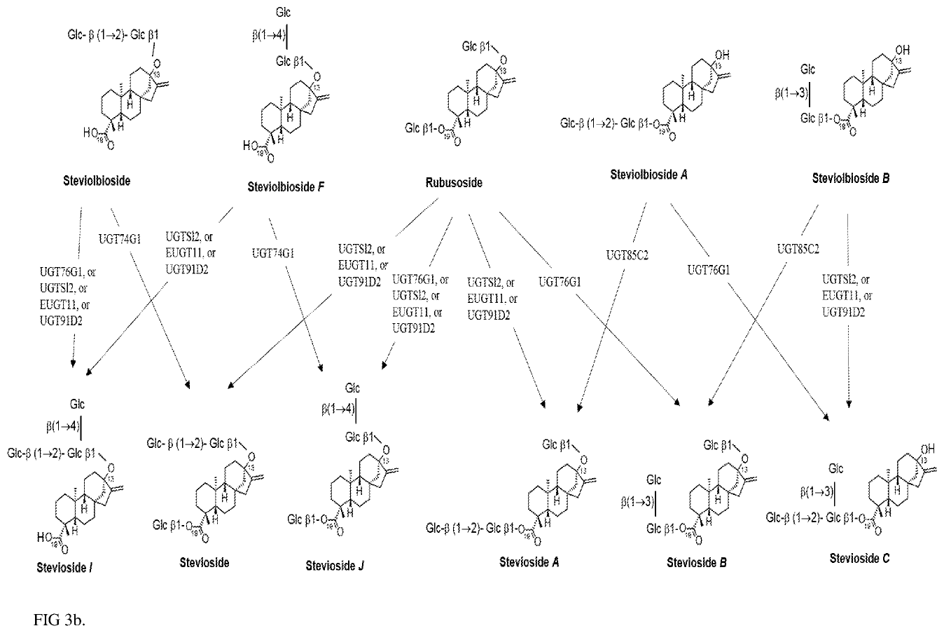

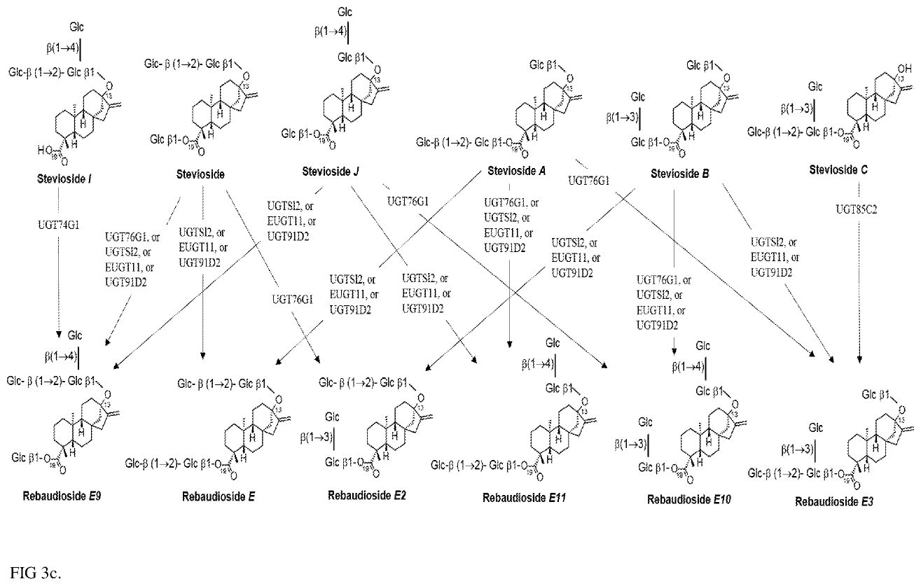

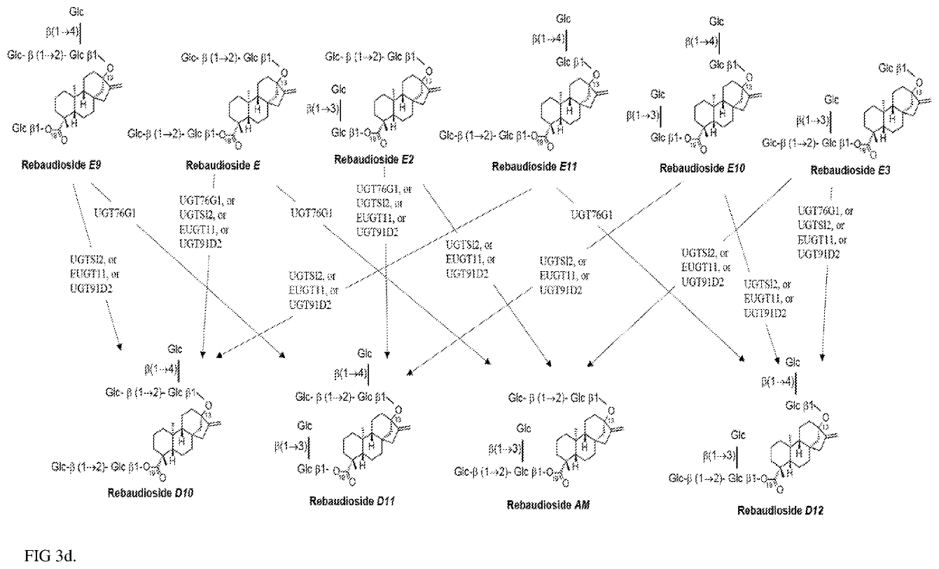

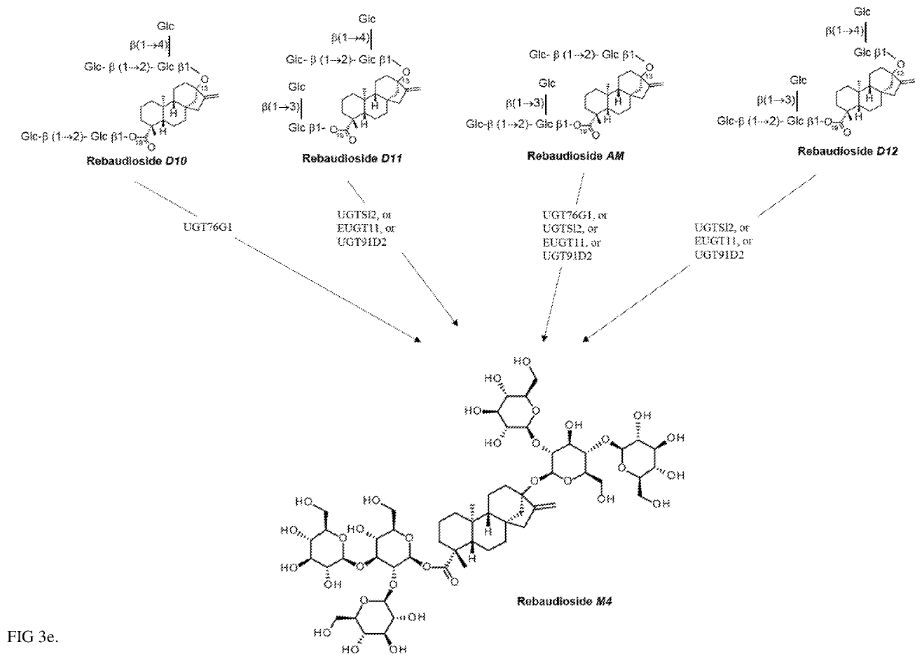

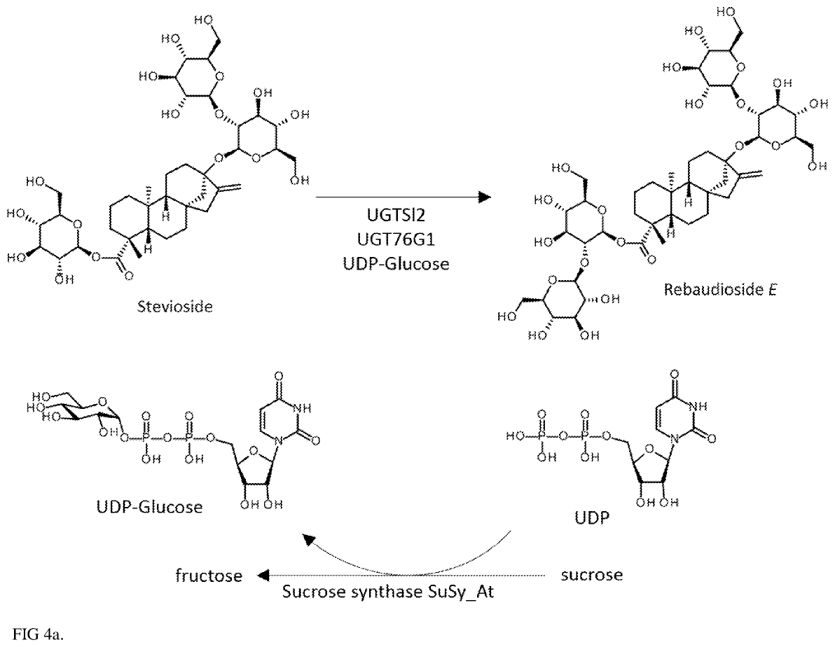

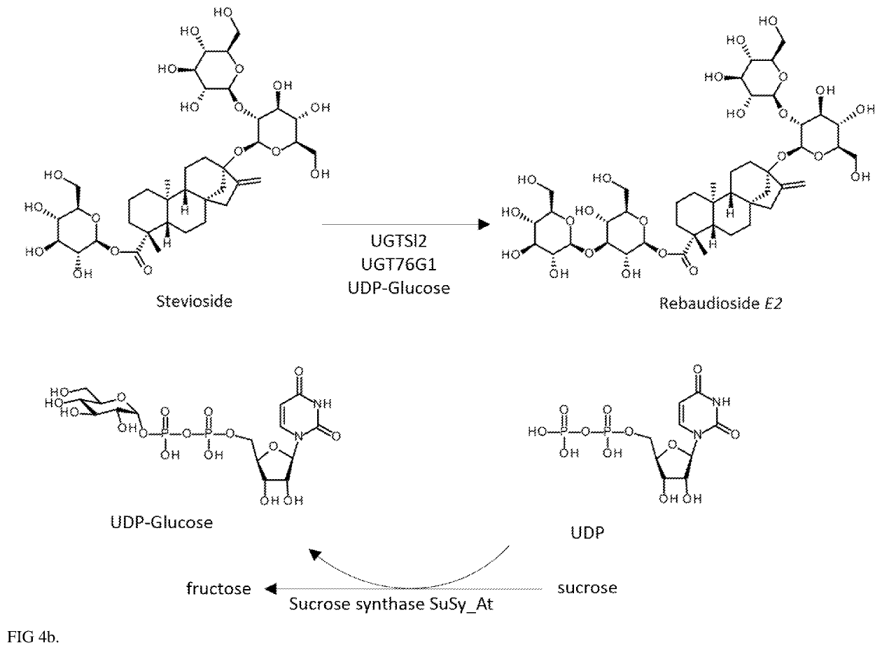

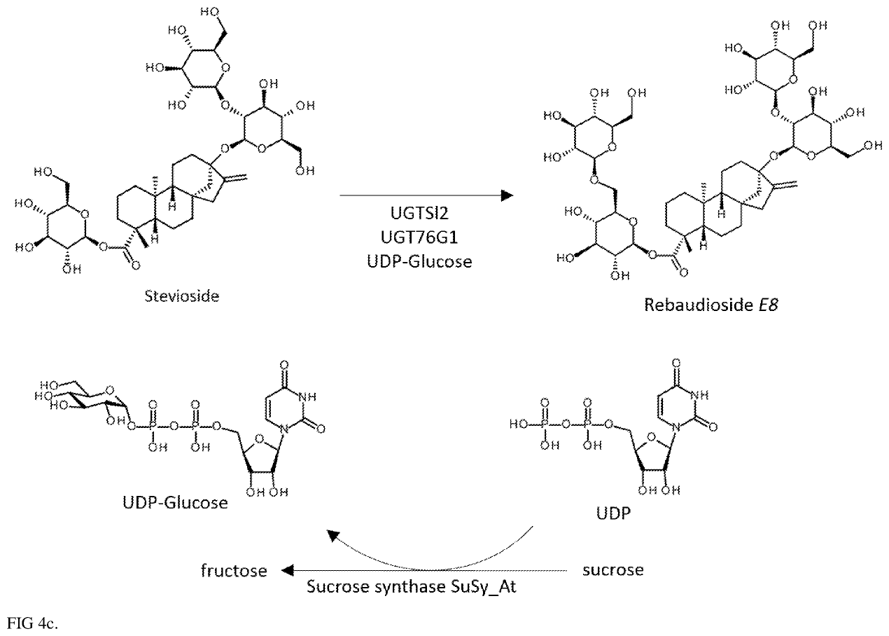

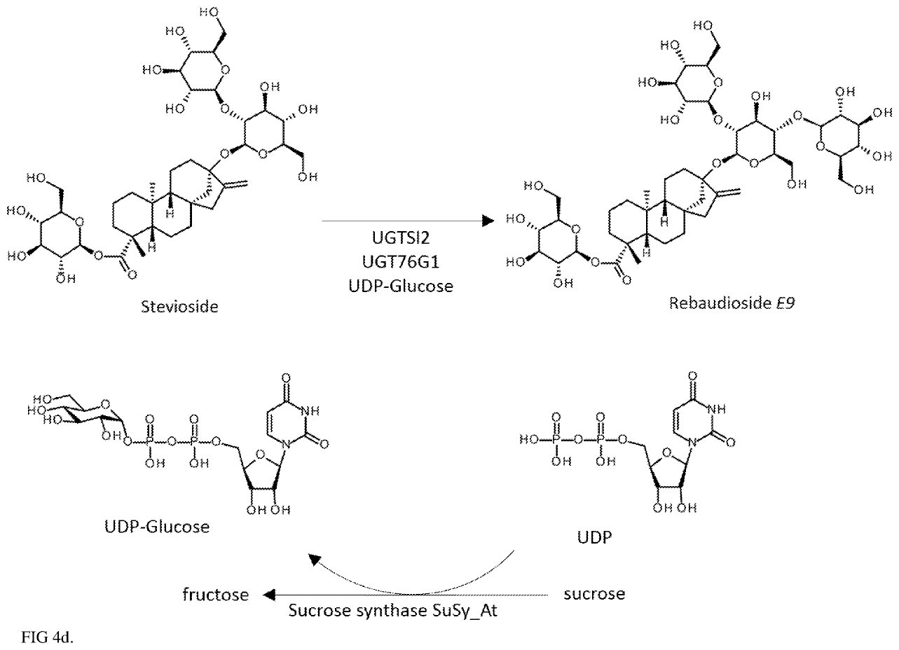

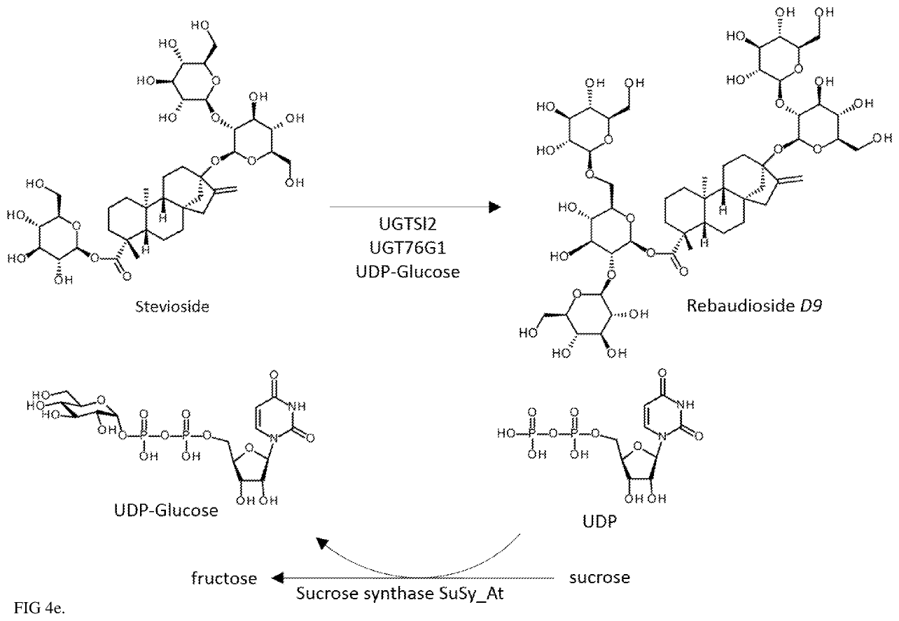

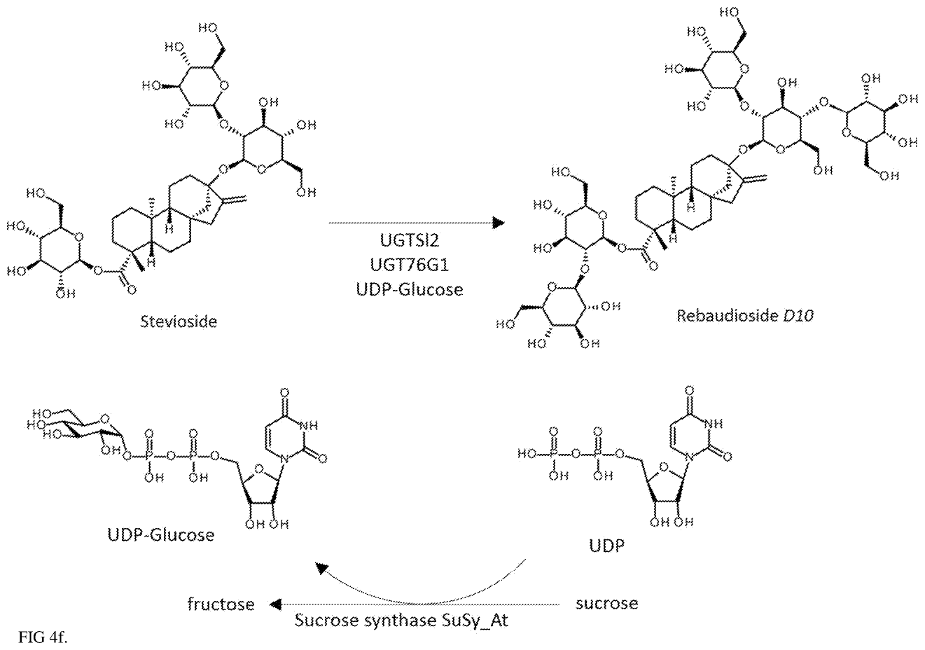

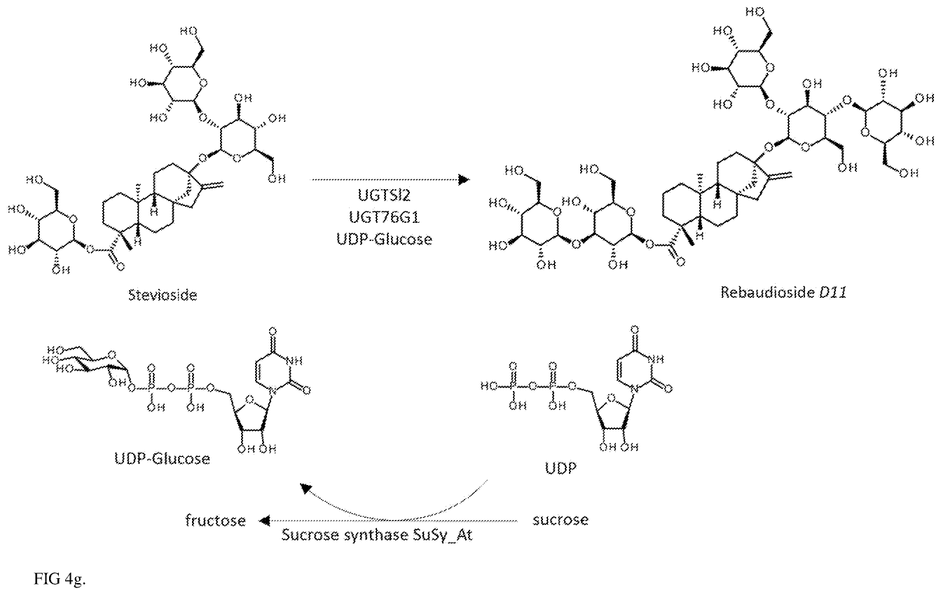

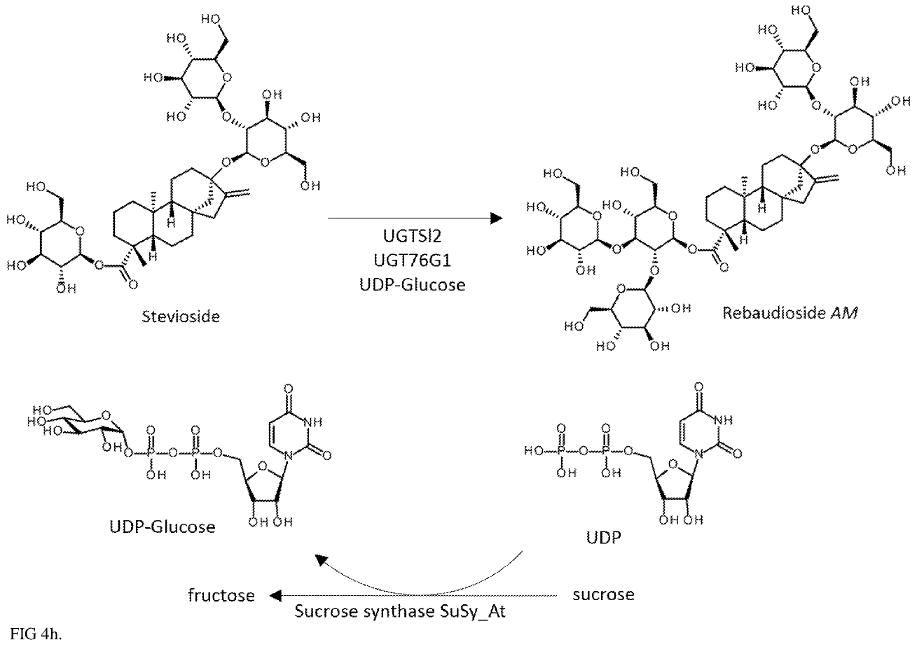

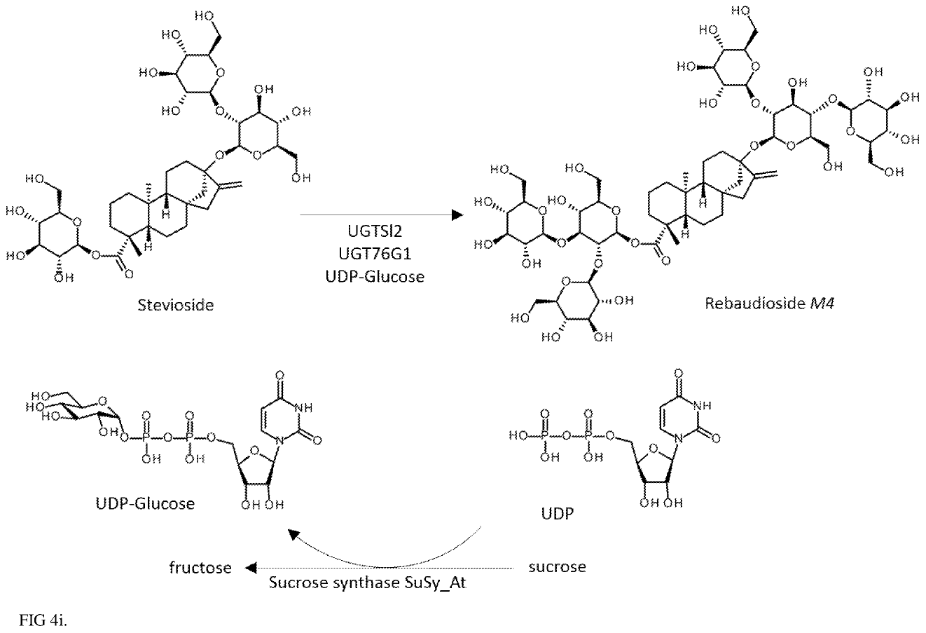

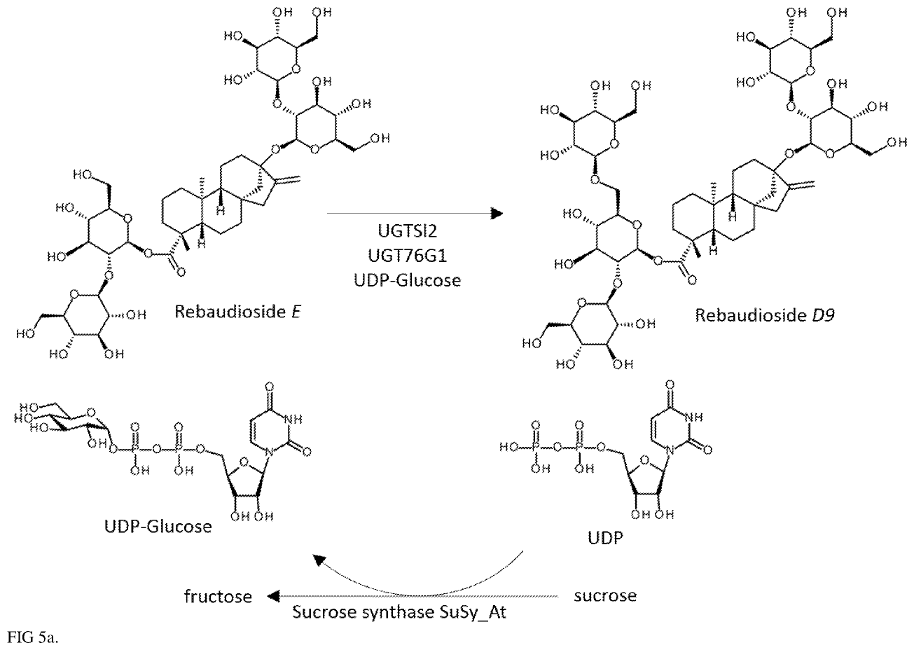

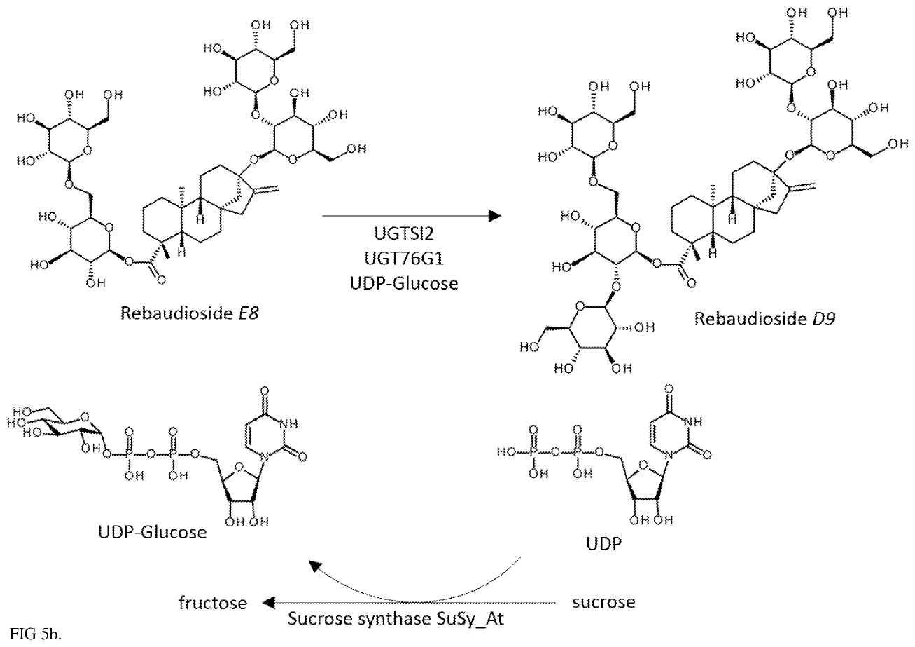

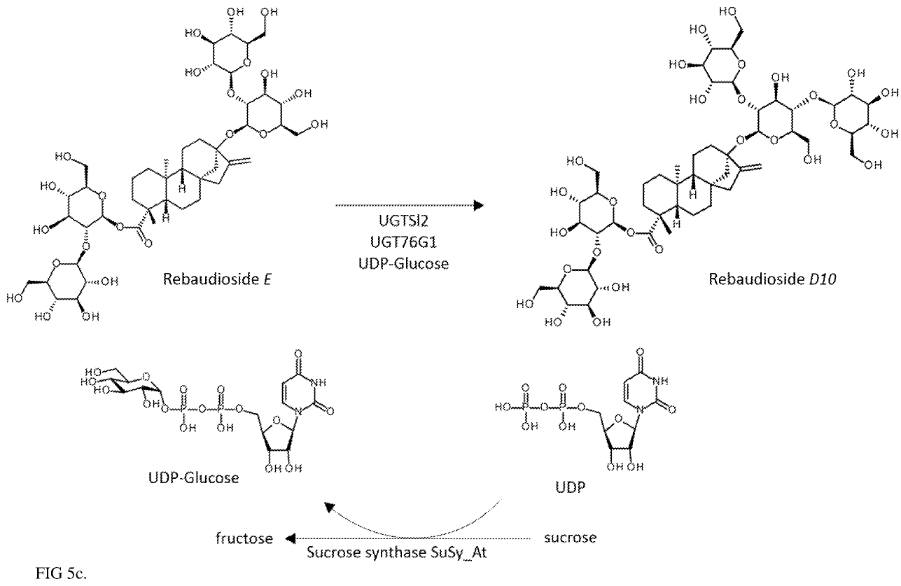

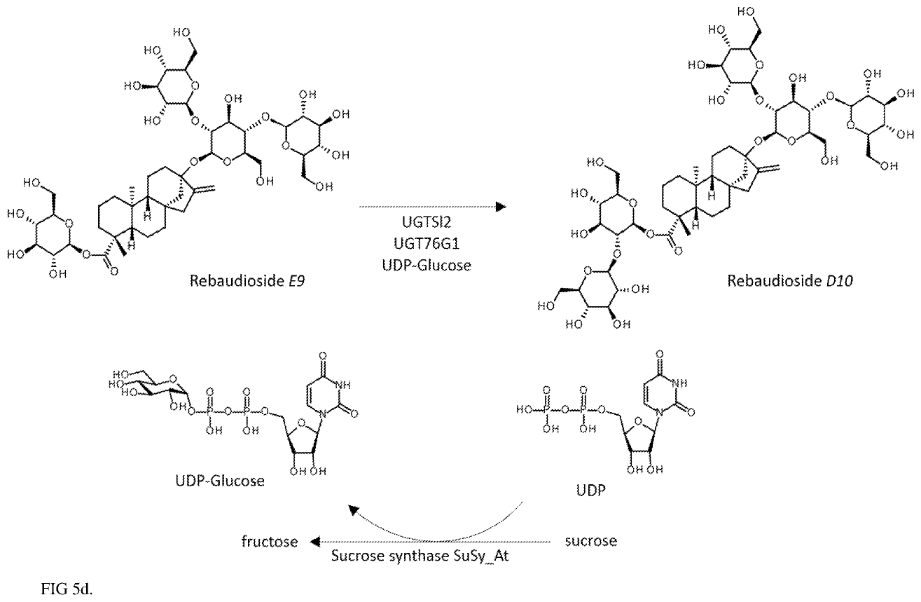

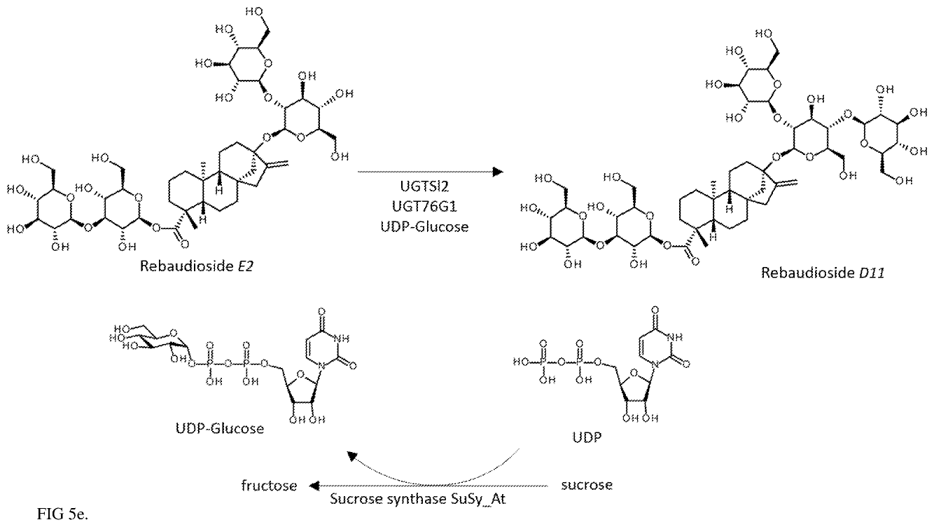

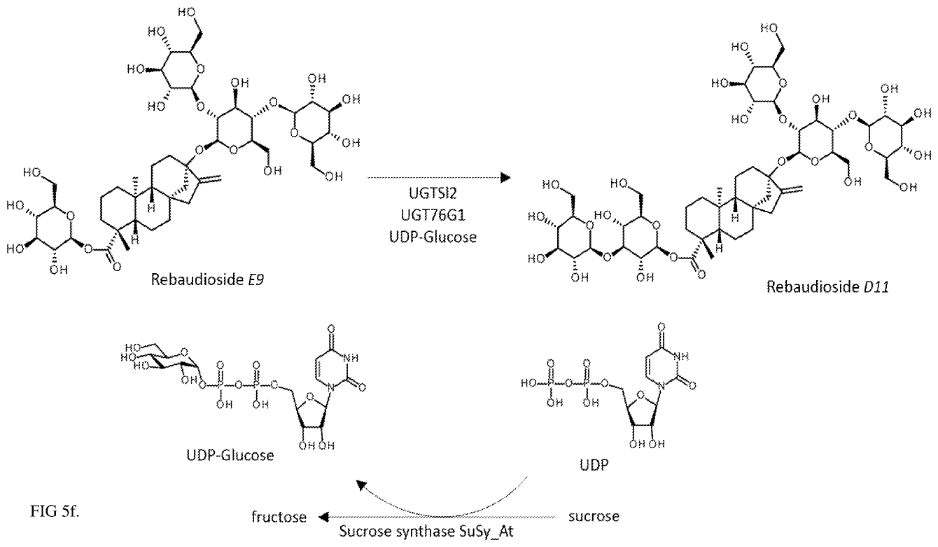

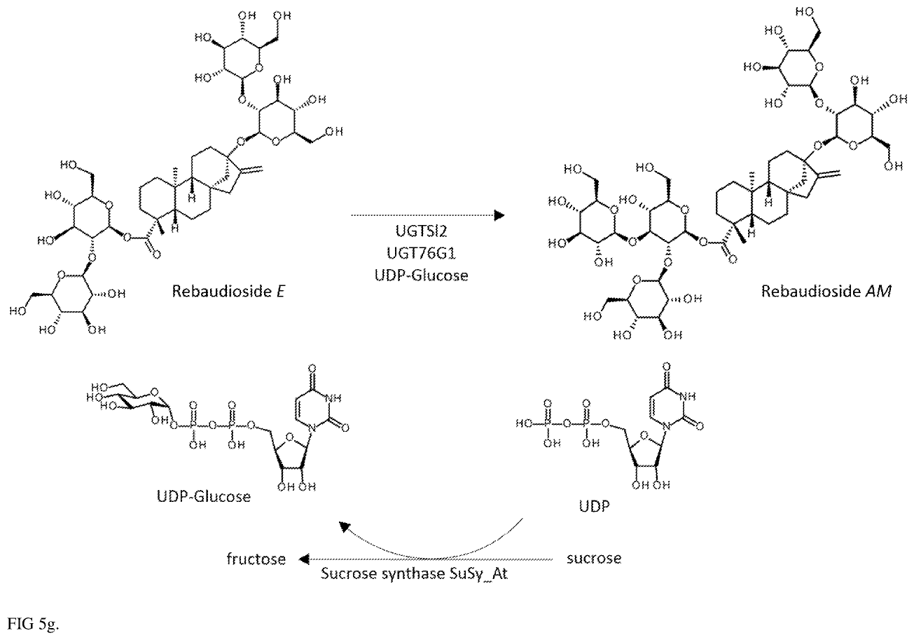

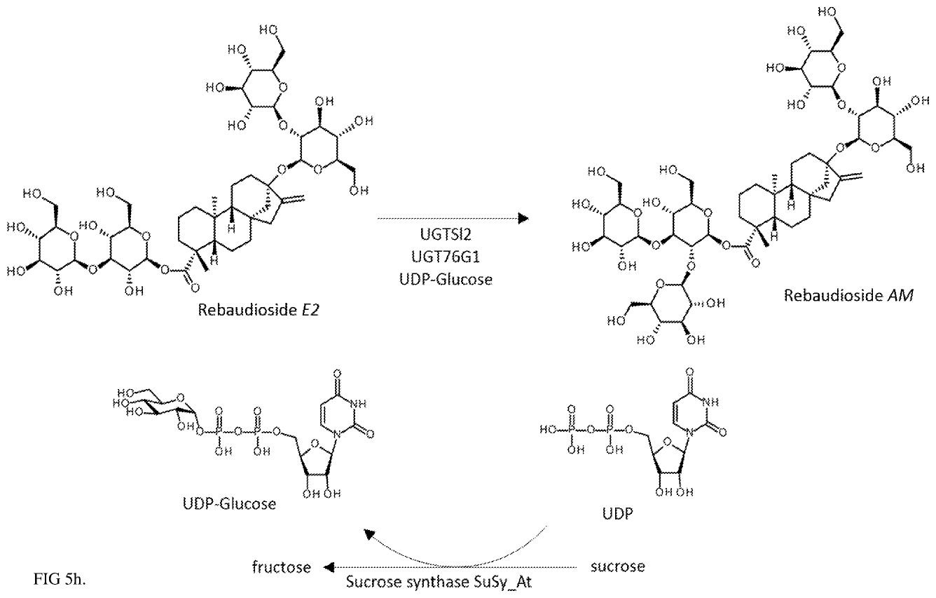

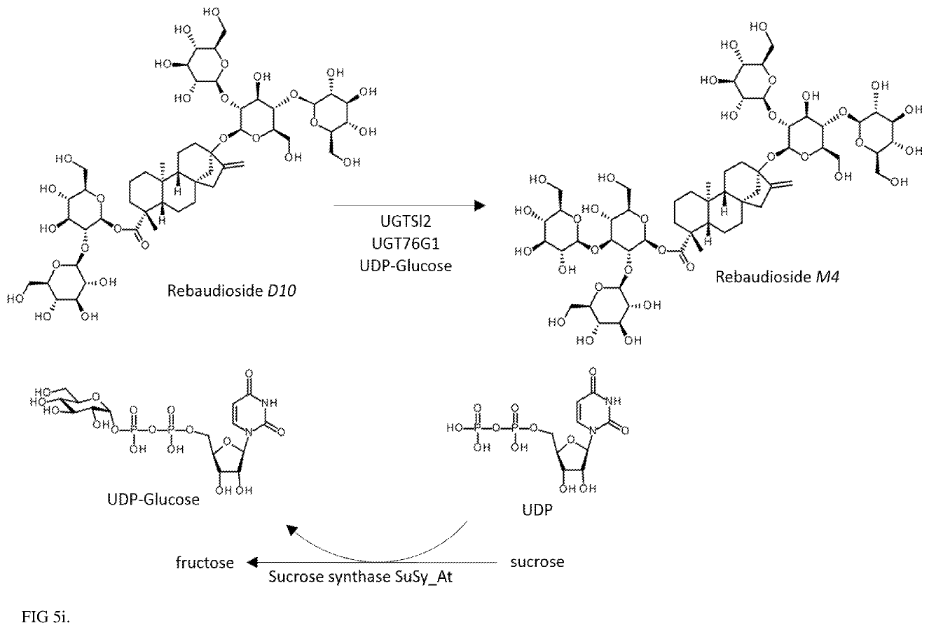

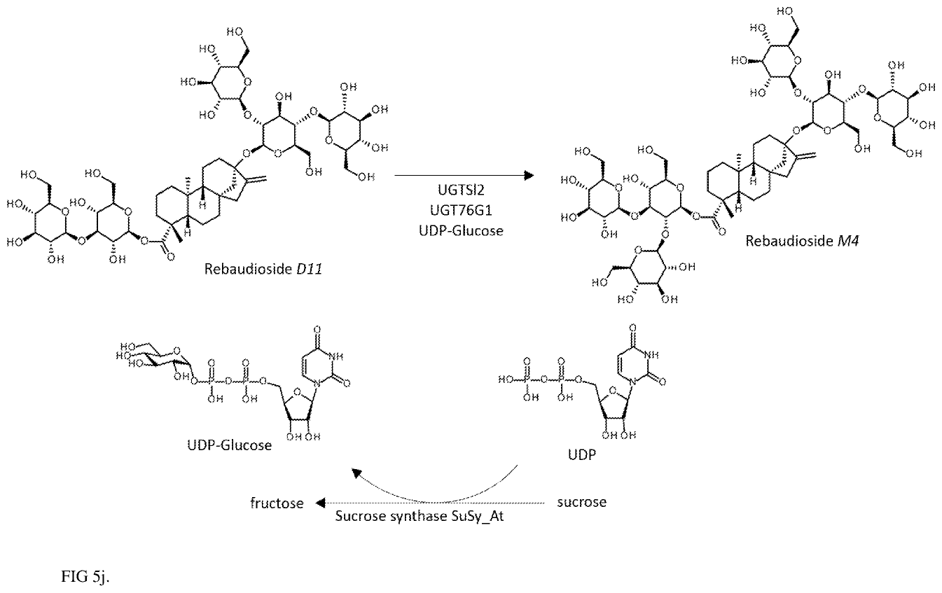

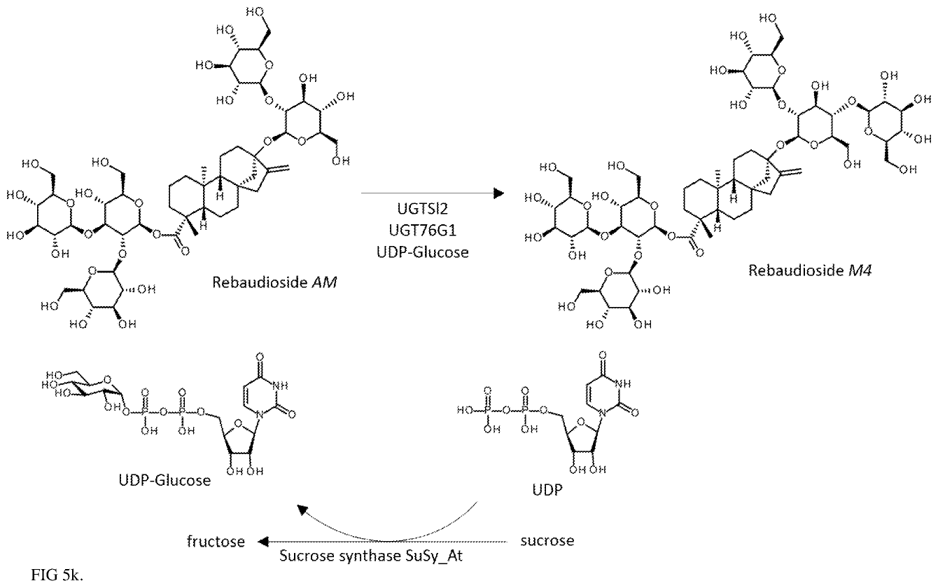

### DETAILED DESCRIPTION

The present invention provides a process for preparing a composition comprising a target steviol glycoside by contacting a starting composition comprising an organic substrate with a microbial cell and/or enzyme preparation, thereby producing a composition comprising a target steviol glycoside.

One object of the invention is to provide an efficient biocatalytic method for preparing target steviol glycosides, particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM, rebaudioside M4, and/or other synthetic steviol glycoside from various starting compositions.

### Starting Composition

As used herein, “starting composition” refers to any composition (generally an aqueous solution) containing one or more organic compound comprising at least one carbon atom.

In one embodiment, the starting composition is selected from the group consisting of steviol, steviol glycosides, polyols and various carbohydrates.

The starting composition steviol glycoside is selected from the group consisting of steviol, steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D10, rebaudioside D11, rebaudioside D12, rebaudioside AM, or steviol glycosides occurring in Stevia rebaudiana plant, synthetic steviol glycosides, e.g. enzymatically glucosylated steviol glycosides, and combinations thereof.

In one embodiment, the starting composition is steviol.

In another embodiment, the starting composition steviol glycoside is steviolmonoside.

In yet another embodiment, the starting composition steviol glycoside is steviolmonoside A.

In another embodiment, the starting composition steviol glycoside is steviolbioside.

In another embodiment, the starting composition steviol glycoside is steviolbioside A.

In another embodiment, the starting composition steviol glycoside is steviolbioside B.

In another embodiment, the starting composition steviol glycoside is steviolbioside F.

In another embodiment, the starting composition steviol glycoside is steviolbioside G.

In another embodiment, the starting composition steviol glycoside is rubusoside.

In another embodiment, the starting composition steviol glycoside is stevioside.

In another embodiment, the starting composition steviol glycoside is stevioside A.

In another embodiment, the starting composition steviol glycoside is stevioside B.

In another embodiment, the starting composition steviol glycoside is stevioside C.

In another embodiment, the starting composition steviol glycoside is stevioside I.

In another embodiment, the starting composition steviol glycoside is stevioside J.

In another embodiment, the starting composition steviol glycoside is stevioside K.

In another embodiment, the starting composition steviol glycoside is stevioside L.

In another embodiment, the starting composition steviol glycoside is rebaudioside E.

In another embodiment, the starting composition steviol glycoside is rebaudioside E2.

In another embodiment, the starting composition steviol glycoside is rebaudioside E3.

In another embodiment, the starting composition steviol glycoside is rebaudioside E8.

In another embodiment, the starting composition steviol glycoside is rebaudioside E9.

In another embodiment, the starting composition steviol glycoside is rebaudioside E10.

In another embodiment, the starting composition steviol glycoside is rebaudioside E11.

In another embodiment, the starting composition steviol glycoside is rebaudioside E12.

In another embodiment, the starting composition steviol glycoside is rebaudioside D10.

In another embodiment, the starting composition steviol glycoside is rebaudioside D11.

In another embodiment, the starting composition steviol glycoside is rebaudioside D12.

In another embodiment, the starting composition steviol glycoside is rebaudioside AM.

The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced.

The term “carbohydrate” refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, psicose, 5-ketofructose, turanose, allose, tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

The starting composition may be synthetic or purified (partially or entirely), commercially available or prepared.

In one embodiment, the starting composition is glycerol.

In another embodiment, the starting composition is glucose.

In another embodiment, the starting composition is rhamnose.

In still another embodiment, the starting composition is sucrose.

In yet another embodiment, the starting composition is starch.

In another embodiment, the starting composition is maltodextrin.

In yet another embodiment, the starting composition is cellulose.

In still another embodiment, the starting composition is amylose.

The organic compound(s) of starting composition serve as a substrate(s) for the production of the target steviol glycoside(s), as described herein.

### Target Steviol Glycoside

The target steviol glycoside of the present method can be any steviol glycoside that can be prepared by the process disclosed herein. In one embodiment, the target steviol glycoside is selected from the group consisting of steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM, rebaudioside M4, or other synthetic steviol glycosides, e.g. enzymatically glucosylated steviol glycosides and combinations thereof.

In one embodiment, the target steviol glycoside is steviolmonoside.

In another embodiment, the target steviol glycoside is steviolmonoside A.

In another embodiment, the target steviol glycoside is steviolbioside.

In another embodiment, the target steviol glycoside is steviolbioside A.

In another embodiment, the target steviol glycoside is steviolbioside B.

In another embodiment, the target steviol glycoside is steviolbioside F.

In another embodiment, the target steviol glycoside is steviolbioside G.

In another embodiment, the target steviol glycoside is rubusoside.

In another embodiment, the target steviol glycoside is stevioside.

In another embodiment, the target steviol glycoside is stevioside A.

In another embodiment, the target steviol glycoside is stevioside B.

In another embodiment, the target steviol glycoside is stevioside C.

In another embodiment, the target steviol glycoside is stevioside L.

In another embodiment, the target steviol glycoside is stevioside J.

In another embodiment, the target steviol glycoside is stevioside K.

In another embodiment, the target steviol glycoside is stevioside L.

In another embodiment, the target steviol glycoside is rebaudioside E.

In another embodiment, the target steviol glycoside is rebaudioside E2.

In another embodiment, the target steviol glycoside is rebaudioside E3.

In another embodiment, the target steviol glycoside is rebaudioside E8.

In another embodiment, the target steviol glycoside is rebaudioside E9.

In another embodiment, the target steviol glycoside is rebaudioside E10.

In another embodiment, the target steviol glycoside is rebaudioside E11.

In another embodiment, the target steviol glycoside is rebaudioside E12.

In another embodiment, the target steviol glycoside is rebaudioside D9.

In another embodiment, the target steviol glycoside is rebaudioside D10.

In another embodiment, the target steviol glycoside is rebaudioside D11.

In another embodiment, the target steviol glycoside is rebaudioside D12.

In another embodiment, the target steviol glycoside is rebaudioside AM.

In another embodiment, the target steviol glycoside is rebaudioside M4.

The target steviol glycoside can be in any polymorphic or amorphous form, including hydrates, solvates, anhydrous or combinations thereof.

In one embodiment, the present invention is a biocatalytic process for the production of steviolmonoside.

In one embodiment, the present invention is a biocatalytic process for the production of steviolmonoside A.

In one embodiment, the present invention is a biocatalytic process for the production of steviolbioside.

In one embodiment, the present invention is a biocatalytic process for the production of steviolbioside A.

In one embodiment, the present invention is a biocatalytic process for the production of steviolbioside B.

In one embodiment, the present invention is a biocatalytic process for the production of steviolbioside F.

In one embodiment, the present invention is a biocatalytic process for the production of steviolbioside G.

In one embodiment, the present invention is a biocatalytic process for the production of rubusoside.

In one embodiment, the present invention is a biocatalytic process for the production of stevioside.

In one embodiment, the present invention is a biocatalytic process for the production of stevioside A.

In one embodiment, the present invention is a biocatalytic process for the production of stevioside B.

In one embodiment, the present invention is a biocatalytic process for the production of stevioside C.

In one embodiment, the present invention is a biocatalytic process for the production of stevioside L In one embodiment, the present invention is a biocatalytic process for the production of stevioside J.

In one embodiment, the present invention is a biocatalytic process for the production of stevioside K.

In one embodiment, the present invention is a biocatalytic process for the production of stevioside L.

In one embodiment, the present invention is a biocatalytic process for the production of rebaudioside E.

In one embodiment, the present invention is a biocatalytic process for the production of rebaudioside E2.

In one embodiment, the present invention is a biocatalytic process for the production of rebaudioside E3.

In one embodiment, the present invention is a biocatalytic process for the production of rebaudioside E8.

In one embodiment, the present invention is a biocatalytic process for the production of rebaudioside E9.

In one embodiment, the present invention is a biocatalytic process for the production of rebaudioside E10.

In one embodiment, the present invention is a biocatalytic process for the production of rebaudioside E11.

In one embodiment, the present invention is a biocatalytic process for the production of rebaudioside E12.

In one embodiment, the present invention is a biocatalytic process for the production of rebaudioside D9.

In one embodiment, the present invention is a biocatalytic process for the production of rebaudioside D10.

In one embodiment, the present invention is a biocatalytic process for the production of rebaudioside D11.

In one embodiment, the present invention is a biocatalytic process for the production of rebaudioside D12.

In one embodiment, the present invention is a biocatalytic process for the production of rebaudioside AM.

In one embodiment, the present invention is a biocatalytic process for the production of rebaudioside M4.

In a particular embodiment, the present invention provides for the biocatalytic process for the production of rebaudioside E from a starting composition comprising stevioside and UDP-glucose.

In a particular embodiment, the present invention provides for the biocatalytic process for the production of rebaudioside E2 from a starting composition comprising stevioside and UDP-glucose.

In a particular embodiment, the present invention provides for the biocatalytic process for the production of rebaudioside E8 from a starting composition comprising stevioside and UDP-glucose.

In a particular embodiment, the present invention provides for the biocatalytic process for the production of rebaudioside E9 from a starting composition comprising stevioside and UDP-glucose.

In a particular embodiment, the present invention provides for the biocatalytic process for the production of rebaudioside D9 from a starting composition comprising stevioside and UDP-glucose.

In a particular embodiment, the present invention provides for the biocatalytic process for the production of rebaudioside D10 from a starting composition comprising stevioside and UDP-glucose.

In a particular embodiment, the present invention provides for the biocatalytic process for the production of rebaudioside D11 from a starting composition comprising stevioside and UDP-glucose.

In a particular embodiment, the present invention provides for the biocatalytic process for the production of rebaudioside AM from a starting composition comprising stevioside and UDP-glucose.

In a particular embodiment, the present invention provides for the biocatalytic process for the production of rebaudioside M4 from a starting composition comprising stevioside and UDP-glucose.

In a particular embodiment, the present invention provides for the biocatalytic process for the production of rebaudioside D9 from a starting composition comprising rebaudioside E and UDP-glucose.

In a particular embodiment, the present invention provides for the biocatalytic process for the production of rebaudioside D9 from a starting composition comprising rebaudioside E8 and UDP-glucose.

In a particular embodiment, the present invention provides for the biocatalytic process for the production of rebaudioside M4 from a starting composition comprising rebaudioside D10 and UDP-glucose.

In a particular embodiment, the present invention provides for the biocatalytic process for the production of rebaudioside M4 from a starting composition comprising rebaudioside D11 and UDP-glucose.

In a particular embodiment, the present invention provides for the biocatalytic process for the production of rebaudioside M4 from a starting composition comprising rebaudioside AM and UDP-glucose.

Optionally, the method of the present invention further comprises separating the target steviol glycoside from the medium to provide a highly purified target steviol glycoside composition. The target steviol glycoside can be separated by any suitable method, such as, for example, crystallization, separation by membranes, centrifugation, extraction, chromatographic separation or a combination of such methods.

In particular embodiments, the process described herein results in a highly purified target steviol glycoside composition. The term “highly purified”, as used herein, refers to a composition having greater than about 80% by weight of the target steviol glycoside on an anhydrous (dried) basis. In one embodiment, the highly purified target steviol glycoside composition contains greater than about 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside E, the process described herein provides a composition having greater than about 80% rebaudioside E content by weight on a dried basis. In another particular embodiment, when the target steviol glycoside is rebaudioside E, the process described herein provides a composition comprising greater than about 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside E2, the process described herein provides a composition having greater than about 80% rebaudioside E2 content by weight on a dried basis. In another particular embodiment, when the target steviol glycoside is rebaudioside E2, the process described herein provides a composition comprising greater than 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside E8, the process described herein provides a composition having greater than about 80% rebaudioside E8 content by weight on a dried basis. In another particular embodiment, when the target steviol glycoside is rebaudioside E8, the process described herein provides a composition comprising greater than 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside E9, the process described herein provides a composition having greater than about 80% rebaudioside E9 content by weight on a dried basis. In another particular embodiment, when the target steviol glycoside is rebaudioside E9, the process described herein provides a composition comprising greater than 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside D9, the process described herein provides a composition having greater than about 80% rebaudioside D9 content by weight on a dried basis. In another particular embodiment, when the target steviol glycoside is rebaudioside D9, the process described herein provides a composition comprising greater 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside D10, the process described herein provides a composition having greater than about 80% rebaudioside D10 content by weight on a dried basis. In another particular embodiment, when the target steviol glycoside is rebaudioside D10, the process described herein provides a composition comprising greater than 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside D11, the process described herein provides a composition having greater than about 80% rebaudioside D11 content by weight on a dried basis. In another particular embodiment, when the target steviol glycoside is rebaudioside D11, the process described herein provides a composition comprising greater than 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside AM, the process described herein provides a composition having greater than about 80% rebaudioside AM content by weight on a dried basis. In another particular embodiment, when the target steviol glycoside is rebaudioside AM, the process described herein provides a composition comprising greater than 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is rebaudioside M4, the process described herein provides a composition having greater than about 80% rebaudioside M4 content by weight on a dried basis. In another particular embodiment, when the target steviol glycoside is rebaudioside M4, the process described herein provides a composition comprising greater than 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

### Microorganisms and Enzyme Preparations

In one embodiment of present invention, a microorganism (microbial cell) and/or enzyme preparation is contacted with a medium containing the starting composition to produce target steviol glycosides.

The enzyme can be provided in the form of a whole cell suspension, a crude lysate, a purified enzyme or a combination thereof. In one embodiment, the biocatalyst is a purified enzyme capable of converting the starting composition to the target steviol glycoside. In another embodiment, the biocatalyst is a crude lysate comprising at least one enzyme capable of converting the starting composition to the target steviol glycoside. In still another embodiment, the biocatalyst is a whole cell suspension comprising at least one enzyme capable of converting the starting composition to the target steviol glycoside.

In another embodiment, the biocatalyst is one or more microbial cells comprising enzyme(s) capable of converting the starting composition to the target steviol glycoside. The enzyme can be located on the surface of the cell, inside the cell or located both on the surface of the cell and inside the cell.

Suitable enzymes for converting the starting composition to target steviol glycosides include, but are not limited to, the steviol biosynthesis enzymes, NDP-glucosyltransferases (NGTs), ADP-glucosyltransferases (AGTs), CDP-glucosyltransferases (CGTs), GDP-glucosyltransferases (GGTs), TDP-glucosyltransferases (TDPs), UDP-glucosyltransferases (UGTs). Optionally it may include NDP-recycling enzyme(s), ADP-recycling enzyme(s), CDP-recycling enzyme(s), GDP-recycling enzyme(s), TDP-recycling enzyme(s), and/or UDP-recycling enzyme(s).

In one embodiment, the steviol biosynthesis enzymes include mevalonate (MVA) pathway enzymes.

In another embodiment, the steviol biosynthesis enzymes include non-mevalonate 2-C-methyl-D-erythritol-4-phosphate pathway (MEP/DOXP) enzymes.

In one embodiment the steviol biosynthesis enzymes are selected from the group including geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase etc.

The UDP-glucosyltransferase can be any UDP-glucosyltransferase capable of adding at least one glucose unit to steviol and/or a steviol glycoside substrate to provide the target steviol glycoside.

In one embodiment, steviol biosynthesis enzymes and UDP-glucosyltransferases are produced in a microbial cell. The microbial cell may be, for example, E. coli, Saccharomyces sp., Aspergillus sp., Pichia sp., Bacillus sp., Yarrowia sp. etc. In another embodiment, the UDP-glucosyltransferases are synthesized.

In one embodiment, the UDP-glucosyltransferase is selected from group including UGT74G1, UGT85C2, UGTS12, UGT76G1, EUGT11, UGT91D2 and UGTs having substantial (>85%, >86%, >87%, >88%, >89%, >90%, >91%, >92%, >93%, >94%, >95%, >96%, >97%, >98%, >99%) amino-acid sequence identity to SEQ ID 2, SEQ ID 3, SEQ ID 4, SEQ ID 5, SEQ ID 6 and SEQ ID 7, respectively as well as isolated nucleic acid molecules that code for these UGTs. Alternative amino-acid sequences can also be obtained through further translocation, inversion, substitution, insertion, deletion and/or duplication of the sequences having substantial (>85%, >86%, >87%, >88%, >89%, >90%, >91%, >92%, >93%, >94%, >95%, >96%, >97%, >98%, >99%) amino-acid sequence identity to these polypeptides.

In one embodiment, steviol biosynthesis enzymes, UGTs and UDP-glucose recycling system are present in one microorganism (microbial cell). The microorganism may be for example, E. coli, Saccharomyces sp., Aspergillus sp., Pichia sp., Bacillus sp., Yarrowia sp.

In one embodiment, the UDP-glucosyltransferase is any UDP-glucosyltransferase capable of adding at least one glucose unit to steviol or any starting steviol glycoside bearing an —OH functional group at C13 to give a target steviol glycoside having an —O-glucose beta glucopyranoside glycosidic linkage at C13. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to steviol or any starting steviol glycoside bearing a —COOH functional group at C19 to give a target steviol glycoside having a —COO-glucose beta-glucopyranoside glycosidic linkage at C19. In a particular embodiment, the UDP-glucosyltransferase is UGT74G1, or a UGT having >85% amino-acid sequence identity with SEQ ID 2.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1->2 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1->3 glucopyranoside glycosidic linkage(s) at the newly formed bond glycosidic bond(s). In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1-4 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C19 side of any starting steviol glycoside to give a target steviol glycoside with at least one additional glucose bearing at least one beta 1->6 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1->2 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1->3 glucopyranoside glycosidic linkage(s) at the newly formed bond glycosidic bond(s). In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1-4 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to any existing glucose on the C13 side of any starting steviol glycoside to give a target steviol glycoside with at least one additional glucose bearing at least one beta 1->6 glucopyranoside glycosidic linkage(s) at the newly formed glycosidic bond(s). In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In one embodiment, the UDP-glucosyltransferase is any UDP-glucosyltransferase capable of adding at least one glucose unit to steviol to form steviolmonoside. In a particular embodiment, the UDP-glucosyltransferase is UGT85C2 or a UGT having >85% amino-acid sequence identity with UGT85C2 or a UGT having >85% amino-acid sequence identity with SEQ ID 3.

In another embodiment, the UDP-glucosyltransferase is any UDP-glucosyltransferase capable of adding at least one glucose unit to steviol to form steviolmonoside A. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to steviolmonoside to form steviolbioside. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to steviolmonoside to form rubusoside. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to steviolmonoside to form steviolbioside F. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to steviolmonoside A to form rubusoside. In a particular embodiment, the UDP-glucosyltransferase is UGT85C2 or a UGT having >85% amino-acid sequence identity with UGT85C2 or a UGT having >85% amino-acid sequence identity with SEQ ID 3.

In another embodiment, the UDP-glucosyltransferase is any UDP-glucosyltransferase capable of adding at least one glucose unit to steviolmonoside A to form steviolbioside A. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to steviolmonoside A to form steviolbioside B. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to steviolmonoside A to form steviolbioside G. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to steviolbioside to form stevioside. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to steviolbioside to form stevioside L In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rubusoside to form stevioside. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rubusoside to form stevioside A. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rubusoside to form stevioside B. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rubusoside to form stevioside J. In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rubusoside to form stevioside L. In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to steviolbioside A to form stevioside A. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to steviolbioside A to form stevioside C. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to steviolbioside A to form stevioside K. In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to steviolbioside B to form stevioside B. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to steviolbioside B to form stevioside C. In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to steviolbioside G to form stevioside K. In a particular embodiment, the UDP-glucosyltransferase is UGTS12, or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to steviolbioside G to form stevioside L. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to stevioside to form rebaudioside E. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside to form rebaudioside E2. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside to form rebaudioside E8. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside to form rebaudioside E9. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside A to form rebaudioside E. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside A to form rebaudioside E3. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside A to form rebaudioside E11. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside A to form rebaudioside E12. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside B to form rebaudioside E2. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside B to form rebaudioside E3. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside B to form rebaudioside E10. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside C to form rebaudioside E3. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to stevioside I to form rebaudioside E9. In a particular embodiment, the UDP-glucosyltransferase is UGT74G1 or a UGT having >85% amino-acid sequence identity with SEQ ID 2.

In another embodiment, the UDP-glucosyltransferase is any UDP-glucosyltransferase capable of adding at least one glucose unit to stevioside J to form rebaudioside E9. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside J to form rebaudioside E10. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside J to form rebaudioside E11. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside K to form rebaudioside E12. In a particular embodiment, the UDP-glucosyltransferase is UGT85C2, or a UGT having >85% amino-acid sequence identity with SEQ ID 3.

In another embodiment, the UDP-glucosyltransferase is any UDP-glucosyltransferase capable of adding at least one glucose unit to stevioside L to form rebaudioside E8. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside L to form rebaudioside E12. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E to form rebaudioside D9. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E to form rebaudioside D10. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside E to form rebaudioside AM. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside E2 to form rebaudioside D11. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside E2 to form rebaudioside AM. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E3 to form rebaudioside D12. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside E3 to form rebaudioside AM. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E8 to form rebaudioside D9. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E9 to form rebaudioside D10. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E9 to form rebaudioside D11. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside E10 to form rebaudioside D11. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E10 to form rebaudioside D12. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E11 to form rebaudioside D10. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside E11 to form rebaudioside D12. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside E12 to form rebaudioside D9. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside D10 to form rebaudioside M4. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to rebaudioside D11 to form rebaudioside M4. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside D12 to form rebaudioside M4. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to rebaudioside AM to form rebaudioside M4. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In a particular embodiment, the UDP-glucosyltransferase is UGT76G1, or a UGT having >85% amino-acid sequence identity with SEQ ID 5.

In another embodiment, the UDP-glucosyltransferase is any UDP-glucosyltransferase capable of adding at least one glucose unit to stevioside to form rebaudioside D9. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7.

In another embodiment, the UDP-glucosyltransferase is any UDP-glucosyltransferase capable of adding at least one glucose unit to stevioside to form rebaudioside M4. In a particular embodiment, the UDP-glucosyltransferase is UGTS12 or a UGT having >85% amino-acid sequence identity with SEQ ID 4. In another particular embodiment, the UDP-glucosyltransferase is EUGT11, or a UGT having >85% amino-acid sequence identity with SEQ ID 6. In yet another particular embodiment, the UDP-glucosyltransferase is UGT91D2, or a UGT having >85% amino-acid sequence identity with SEQ ID 7. In another particular embodiment, the UDP-glucosyltransferase is UGT76G1, or a UGT having >85% amino-acid sequence identity with SEQ ID 5.

Optionally, the method of the present invention further comprises recycling UDP to provide UDP-glucose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-glucosyltransferase and UDP-glucose. The UDP recycling enzyme can be sucrose synthase SuSy_At or a sucrose synthase having >85% amino-acid sequence identity with SuSy_At and the recycling substrate can be sucrose.

In one embodiment, the recycling catalyst is sucrose synthase SuSy_At or a sucrose synthase having >85% amino-acid sequence identity with SuSy_At.

In one embodiment, the recycling substrate for UDP-glucose recycling catalyst is sucrose.

Optionally, the method of the present invention further comprises the use of transglycosidases that use oligo- or poly-saccharides as the sugar donor to modify recipient target steviol glycoside molecules. Non-limiting examples include cyclodextrin glycosyltransferase (CGTase), fructofuranosidase, amylase, saccharase, glucosucrase, beta-h-fructosidase, beta-fructosidase, sucrase, fructosylinvertase, alkaline invertase., acid invertase, fructofuranosidase. In some embodiments, glucose and sugar(s) other than glucose, including but not limited to fructose, xylose, rhamnose, arabinose, deoxyglucose, galactose are transferred to the recipient target steviol glycosides. In one embodiment, the recipient steviol glycoside is rebaudioside E. In another embodiment, the recipient steviol glycoside is rebaudioside E2. In another embodiment, the recipient steviol glycoside is rebaudioside E8. In another embodiment, the recipient steviol glycoside is rebaudioside E9. In yet another embodiment, the recipient steviol glycoside is rebaudioside D9. In another embodiment, the recipient steviol glycoside is rebaudioside D10. In another embodiment, the recipient steviol glycoside is rebaudioside D11. In another embodiment, the recipient steviol glycoside is rebaudioside AM. In yet another embodiment, the recipient steviol glycoside is rebaudioside M4.

One embodiment of the present invention is a microbial cell comprising an enzyme, i.e. an enzyme capable of converting the starting composition to the target steviol glycoside. Accordingly, some embodiments of the present method include contacting a microorganism with a medium containing the starting composition to provide a medium comprising at least one target steviol glycoside.

The microorganism can be any microorganism possessing the necessary enzyme(s) for converting the starting composition to target steviol glycoside(s). These enzymes are encoded within the microorganism's genome.

Suitable microorganisms include, but are not limited to, E. coli, Saccharomyces sp., Aspergillus sp., Pichia sp., Bacillus sp., Yarrowia sp. etc.

In one embodiment, the microorganism is free when contacted with the starting composition.

In another embodiment, the microorganism is immobilized when contacted with the starting composition. For example, the microorganism may be immobilized to a solid support made from inorganic or organic materials. Non-limiting examples of solid supports suitable to immobilize the microorganism include derivatized cellulose or glass, ceramics, metal oxides or membranes. The microorganism may be immobilized to the solid support, for example, by covalent attachment, adsorption, cross-linking, entrapment or encapsulation.

In still another embodiment, the enzyme capable of converting the starting composition to the target steviol glycoside is secreted out of the microorganism and into the reaction medium.

The target steviol glycoside is optionally purified. Purification of the target steviol glycoside from the reaction medium can be achieved by at least one suitable method to provide a highly purified target steviol glycoside composition. Suitable methods include crystallization, separation by membranes, centrifugation, extraction (liquid or solid phase), chromatographic separation, HPLC (preparative or analytical) or a combination of such methods.

### Uses

Highly purified target glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained according to this invention can be used “as-is” or in combination with other sweeteners, flavors, flavor stabilizers, flavorings with modifying properties (FMP), foaming suppressors, solubility enhancing agents, food ingredients, salts thereof and combinations thereof.

Non-limiting examples of sweeteners include, but are not limited to, steviol glycosides, carbohydrates, psicose, 5-ketofructose, tagatose, polyols, sugar alcohols, natural high intensity sweeteners, synthetic high intensity sweeteners, reduced calorie sweeteners, mogrosides, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin, monatin salts, other indole derivative sweeteners, selligueain A, hematoxylin, monellin, osladin, pterocaryoside A, pterocaryoside B, mabinlin, pentadin, miraculin, curculin, neoculin, chlorogenic acid, cynarin, Luo Han Guo sweetener, mogroside V, siamenoside, siratose, salts thereof, and combinations thereof.

Non-limiting examples of flavors include, but are not limited to, lime, lemon, orange, fruit, banana, grape, pear, pineapple, mango, berry, bitter almond, cola, cinnamon, sugar, cotton candy, vanilla, other compounds listed in FEMA (Flavor Extract Manufacturers Association) flavoring substances GRAS lists, salts thereof, and combinations thereof.

Non-limiting examples of other food ingredients include, but are not limited to, acidulants, organic and amino acids, coloring agents, bulking agents, modified starches, gums, texturizers, preservatives, caffeine, color stabilizers, flavor stabilizers, natural sweetener suppressors, additives, antioxidants, emulsifiers, stabilizers, thickeners, gelling agents, physiologically active substances, functional ingredients, salts thereof, and combinations thereof.

Highly purified target glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained according to this invention can be prepared in various polymorphic forms, including but not limited to hydrates, solvates, anhydrous, amorphous forms and combinations thereof.

Highly purified target glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained according to this invention may be incorporated as a high intensity natural sweetener in foodstuffs, beverages, pharmaceutical compositions, cosmetics, chewing gums, table top products, cereals, dairy products, toothpastes and other oral cavity compositions, etc.

Highly purified target glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained according to this invention may be incorporated as a flavor stabilizer in foodstuffs, beverages, pharmaceutical compositions, cosmetics, chewing gums, table top products, cereals, dairy products, toothpastes and other oral cavity compositions, etc.

Highly purified target glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained according to this invention may be incorporated as a flavoring with modifying properties (FMP) in foodstuffs, beverages, pharmaceutical compositions, cosmetics, chewing gums, table top products, cereals, dairy products, toothpastes and other oral cavity compositions, etc.

Highly purified target glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained according to this invention may be incorporated as a foam stabilizer in foodstuffs, beverages, pharmaceutical compositions, cosmetics, chewing gums, table top products, cereals, dairy products, toothpastes and other oral cavity compositions, etc.

Highly purified target glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained according to this invention may be incorporated as a solubility enhancing agent in foodstuffs, beverages, pharmaceutical compositions, cosmetics, chewing gums, table top products, cereals, dairy products, toothpastes and other oral cavity compositions, etc.

In some embodiments, the highly purified target glycoside(s) particularly, steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 of present invention are present in consumable products, foodstuffs, beverages, pharmaceutical compositions, cosmetics, chewing gums, table top products, cereals, dairy products, toothpastes, other oral cavity compositions, alcoholic beverages such as vodka, wine, beer, liquor, and sake, etc., natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, frozen beef, frozen pork, frozen goat, frozen lamb, frozen mutton, frozen poultry like frozen chicken, frozen duck and frozen turkey, frozen venison, frozen fish, frozen crustaceans like frozen crab and frozen lobster, frozen molluscs like frozen clams, frozen oysters, frozen scallops, and frozen mussels, frozen shrimps, frozen octopus, frozen squid, fresh beef, fresh pork, fresh goat, fresh lamb, fresh mutton, fresh poultry like fresh chicken, fresh duck and fresh turkey, fresh venison, fresh fish, fresh crustaceans like fresh crab and fresh lobster, fresh molluscs like fresh clams, fresh oysters, fresh scallops, and fresh mussels, fresh shrimps, fresh octopus, fresh squid, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, medicinal products, lipsticks, etc at a concentration from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

In one embodiment, the sweetener is present in the beverage in an amount from about 0.0001% by weight to about 8% by weight, such as for example, from about 0.0001% by weight to about 0.0005% by weight, from about 0.0005% by weight to about 0.001% by weight, from about 0.001% by weight to about 0.005% by weight, from about 0.005% by weight to about 0.01% by weight, from about 0.01% by weight to about 0.05% by weight, from about 0.05% by weight to about 0.1% by weight, from about 0.1% by weight to about 0.5% by weight, from about 0.5% by weight to about 1% by weight, from about 1% by weight to about 2% by weight, from about 2% by weight to about 3% by weight, from about 3% by weight to about 4% by weight, from about 4% by weight to about 5% by weight, from about 5% by weight to about 6% by weight, from about 6% by weight to about 7% by weight, and from about 7% by weight to about 8% by weight.

Highly purified target glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 and/or combinations thereof, obtained according to this invention, may be employed as a sweetening compound as the sole sweetener, or it may be used together with at least one high intensity sweeteners such as dulcoside A, dulcoside B, dulcoside C, dulcoside D, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f, rebaudioside 2g, rebaudioside 2h, rebaudioside 2i, rebaudioside 2j, rebaudioside 2k, rebaudioside 21, rebaudioside 2m, rebaudioside 2n, rebaudioside 2o, rebaudioside 2p, rebaudioside 2q, rebaudioside 2r, rebaudioside 2s, rebaudioside A, rebaudioside AIG, rebaudioside A2, rebaudioside A3, rebaudioside A4, rebaudioside AM, rebaudioside B, rebaudioside B2, rebaudioside C, rebaudioside C2, rebaudioside C3, rebaudioside C4, rebaudioside C5, rebaudioside C6, rebaudioside C7, rebaudioside D, rebaudioside D2, rebaudioside D3, rebaudioside D4, rebaudioside D5, rebaudioside D6, rebaudioside D7, rebaudioside D8, rebaudioside D9, rebaudioside D10, rebaudioside D11, rebaudioside D12, rebaudioside D13, rebaudioside E, rebaudioside E2, rebaudioside E3, rebaudioside E4, rebaudioside E5, rebaudioside E6, rebaudioside E7, rebaudioside E8, rebaudioside E9, rebaudioside E10, rebaudioside E11, rebaudioside E12, rebaudioside E13, rebaudioside F, rebaudioside F1, rebaudioside F2, rebaudioside F3, rebaudioside G, rebaudioside H, rebaudioside H1, rebaudioside H2, rebaudioside H3, rebaudioside H4, rebaudioside H5, rebaudioside H6, rebaudioside H7, rebaudioside I, rebaudioside I2, rebaudioside I3, rebaudioside IX, rebaudioside IXa, rebaudioside IXb, rebaudioside IXc, rebaudioside IXd, rebaudioside J, rebaudioside K, rebaudioside K2, rebaudioside KA, rebaudioside L, rebaudioside L1, rebaudioside M, rebaudioside M2, rebaudioside M3, rebaudioside M4, rebaudioside M5, rebaudioside N, rebaudioside N2, rebaudioside N3, rebaudioside N4, rebaudioside N5, rebaudioside N6, rebaudioside N7, rebaudioside O, rebaudioside O2, rebaudioside O3, rebaudioside O4, rebaudioside O5, rebaudioside O6, rebaudioside O7, rebaudioside Q, rebaudioside Q2, rebaudioside Q3, rebaudioside R, rebaudioside R1, rebaudioside S, rebaudioside T, rebaudioside T1, rebaudioside U, rebaudioside U2, rebaudioside U3, rebaudioside V, rebaudioside V2, rebaudioside VIII, rebaudioside VIIIa, rebaudioside VIIIb, rebaudioside W, rebaudioside W2, rebaudioside W3, rebaudioside WB1, rebaudioside WB2, rebaudioside Y, rebaudioside Z1, rebaudioside Z2, rubusoside, steviolbioside, steviolbioside A, steviolbioside B, steviolbioside C, steviolbioside D, steviolbioside E, steviolbioside F, steviolbioside G, steviolmonoside, steviolmonoside A, stevioside, stevioside A, stevioside B, stevioside C, stevioside D, stevioside E, stevioside E2, stevioside F, stevioside G, stevioside H, stevioside I, stevioside J, stevioside K, stevioside L, stevioside M, SvG7, NSF-02, carbohydrates, psicose, 5-ketofructose, tagatose, allose, erythritol, polyols, sugar alcohols, natural high intensity sweeteners, synthetic high intensity sweeteners, reduced calorie sweeteners, mogrosides, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin, monatin salts, other indole derivative sweeteners, selligueain A, hematoxylin, monellin, osladin, pterocaryoside A, pterocaryoside B, mabinlin, pentadin, miraculin, curculin, neoculin, chlorogenic acid, cynarin, Luo Han Guo sweetener, mogroside V, siamenoside, siratose, salts thereof, and combinations thereof.

In one embodiment, steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E1, rebaudioside E12, rebaudioside D9, rebaudioside D10, rebaudioside D11, rebaudioside D12, rebaudioside AM and/or rebaudioside M4 can be used in a sweetener composition comprising a compound selected from the group consisting of dulcoside A, dulcoside B, dulcoside C, dulcoside D, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 11, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f, rebaudioside 2g, rebaudioside 2h, rebaudioside 2i, rebaudioside 2j, rebaudioside 2k, rebaudioside 21, rebaudioside 2m, rebaudioside 2n, rebaudioside 2o, rebaudioside 2p, rebaudioside 2q, rebaudioside 2r, rebaudioside 2s, rebaudioside A, rebaudioside AIG, rebaudioside A2, rebaudioside A3, rebaudioside A4, rebaudioside AM, rebaudioside B, rebaudioside B2, rebaudioside C, rebaudioside C2, rebaudioside C3, rebaudioside C4, rebaudioside C5, rebaudioside C6, rebaudioside C7, rebaudioside D, rebaudioside D2, rebaudioside D3, rebaudioside D4, rebaudioside D5, rebaudioside D6, rebaudioside D7, rebaudioside D8, rebaudioside D9, rebaudioside D10, rebaudioside D11, rebaudioside D12, rebaudioside D13, rebaudioside E, rebaudioside E2, rebaudioside E3, rebaudioside E4, rebaudioside E5, rebaudioside E6, rebaudioside E7, rebaudioside E8, rebaudioside E9, rebaudioside E10, rebaudioside E11, rebaudioside E12, rebaudioside E13, rebaudioside F, rebaudioside F1, rebaudioside F2, rebaudioside F3, rebaudioside G, rebaudioside H, rebaudioside H1, rebaudioside H2, rebaudioside H3, rebaudioside H4, rebaudioside H5, rebaudioside H6, rebaudioside H7, rebaudioside I, rebaudioside I2, rebaudioside I3, rebaudioside IX, rebaudioside IXa, rebaudioside IXb, rebaudioside IXc, rebaudioside IXd, rebaudioside J, rebaudioside K, rebaudioside K2, rebaudioside KA, rebaudioside L, rebaudioside L1, rebaudioside M, rebaudioside M2, rebaudioside M3, rebaudioside M4, rebaudioside M5, rebaudioside N, rebaudioside N2, rebaudioside N3, rebaudioside N4, rebaudioside N5, rebaudioside N6, rebaudioside N7, rebaudioside O, rebaudioside O2, rebaudioside O3, rebaudioside O4, rebaudioside O5, rebaudioside O6, rebaudioside O7, rebaudioside Q, rebaudioside Q2, rebaudioside Q3, rebaudioside R, rebaudioside R1, rebaudioside S, rebaudioside T, rebaudioside T1, rebaudioside U, rebaudioside U2, rebaudioside U3, rebaudioside V, rebaudioside V2, rebaudioside VIII, rebaudioside VIIIa, rebaudioside VIIIb, rebaudioside W, rebaudioside W2, rebaudioside W3, rebaudioside WB1, rebaudioside WB2, rebaudioside Y, rebaudioside Z1, rebaudioside Z2, rubusoside, steviolbioside, steviolbioside A, steviolbioside B, steviolbioside C, steviolbioside D, steviolbioside E, steviolbioside F, steviolbioside G, steviolmonoside, steviolmonoside A, stevioside, stevioside A, stevioside B, stevioside C, stevioside D, stevioside E, stevioside E2, stevioside F, stevioside G, stevioside H, stevioside I, stevioside J, stevioside K, stevioside L, stevioside M, SvG7, NSF-02, carbohydrates, psicose, 5-ketofructose, tagatose, allose, erythritol, polyols, sugar alcohols, natural high intensity sweeteners, synthetic high intensity sweeteners, reduced calorie sweeteners, mogrosides, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin, monatin salts, other indole derivative sweeteners, selligueain A, hematoxylin, monellin, osladin, pterocaryoside A, pterocaryoside B, mabinlin, pentadin, miraculin, curculin, neoculin, chlorogenic acid, cynarin, Luo Han Guo sweetener, mogroside V, siamenoside, siratose, salts thereof, and combinations thereof.

Highly purified target glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 may also be used in combination with synthetic high intensity sweeteners such as sucralose, potassium acesulfame, aspartame, alitame, saccharin, neohesperidin dihydrochalcone, cyclamate, neotame, dulcin, suosan advantame, salts thereof, and combinations thereof.

Moreover, highly purified target steviol glycoside(s) particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 can be used in combination with natural sweetener suppressors such as gymnemic acid, hodulcin, ziziphin, lactisole, and others. Steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 may also be combined with various umami taste enhancers. Steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 can be mixed with umami tasting and sweet amino acids such as aspartic acid, glycine, alanine, threonine, proline, serine, glutamate, lysine, tryptophan, salts thereof and combinations thereof.

Highly purified target steviol glycoside(s) particularly, steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 can be used in combination with one or more additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers, salts thereof and combinations thereof.

Highly purified target steviol glycoside(s) particularly, steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 may be combined with polyols or sugar alcohols. The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced which do not adversely affect the taste of the sweetener composition.

Highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 may be combined with reduced calorie sweeteners such as, for example, D-psicose, 5-ketofructose, D-tagatose, L-sugars, L-sorbose, L-arabinose and combinations thereof.

Highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 may also be combined with various carbohydrates. The term “carbohydrate” generally refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, psicose, 5-ketofructose, turanose, allose, tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

Highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained according to this invention can be used in combination with various physiologically active substances or functional ingredients. Functional ingredients generally are classified into categories such as carotenoids, dietary fiber, fatty acids, saponins, antioxidants, nutraceuticals, flavonoids, isothiocyanates, phenols, plant sterols and stanols (phytosterols and phytostanols, polyols, prebiotics, probiotics, postbiotics, phytoestrogens, soy protein, sulfides/thiols, amino acids, proteins, vitamins, and minerals. Functional ingredients also may be classified based on their health benefits, such as cardiovascular, cholesterol-reducing, and anti-inflammatory. Exemplary functional ingredients are provided in WO2013/096420, the contents of which is hereby incorporated by reference.

Highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained according to this invention may be applied as a high intensity sweetener to produce zero calorie, reduced calorie or diabetic beverages and food products with improved taste characteristics. It may also be used in drinks, foodstuffs, pharmaceuticals, and other products in which sugar cannot be used. In addition, highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 can be used as a sweetener not only for drinks, foodstuffs, and other products dedicated for human consumption, but also in animal feed and fodder with improved characteristics.

Highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained according to this invention may be applied as a flavor stabilizer to produce zero calorie, reduced calorie or diabetic beverages and food products with improved flavor stability compared to a control product that does not contain the glycoside(s). It may also be used in drinks, foodstuffs, pharmaceuticals, and other products in which flavor stabilization is preferred. In addition, highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 can be used as a flavor stabilizer not only for drinks, foodstuffs, and other products dedicated for human consumption, but also in animal feed and fodder with improved characteristics.

Highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained according to this invention may be applied as a flavoring with modifying properties (FMP) to produce zero calorie, reduced calorie or diabetic beverages and food products with modified (including enhanced or suppressed) flavor and/or taste profile. It may also be used in drinks, foodstuffs, pharmaceuticals, and other products in which modification (including enhancing or suppressing) of flavor and/or taste profile is preferred. In addition, highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 can be used as a flavoring with modifying properties (FMP) not only for drinks, foodstuffs, and other products dedicated for human consumption, but also in animal feed and fodder with improved characteristics.

Highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained according to this invention may be applied as a foaming suppressor to produce zero calorie, reduced calorie or diabetic beverages and food products with suppressed foaming. It may also be used in drinks, foodstuffs, pharmaceuticals, and other products in which foaming suppression is preferred. In addition, highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 can be used as a foaming suppressor not only for drinks, foodstuffs, and other products dedicated for human consumption, but also in animal feed and fodder with improved characteristics.

Highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained according to this invention may be applied as a solubility enhancing agent to produce zero calorie, reduced calorie or diabetic beverages and food products having less insoluble material compared to a control product that does not contain the glycoside(s). It may also be used in drinks, foodstuffs, pharmaceuticals, and other products in which solubility enhancement of insoluble material is preferred. In addition, highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 can be used as a solubility enhancing agent not only for drinks, foodstuffs, and other products dedicated for human consumption, but also in animal feed and fodder with improved characteristics.

Examples of consumable products in which highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained according to this invention may be used as a sweetening compound, flavor stabilizer, flavoring with modifying properties (FMP), foaming suppressor and/or solubility enhancing agent include, but are not limited to, consumable products, foodstuffs, beverages, pharmaceutical compositions, cosmetics, chewing gums, table top products, cereals, dairy products, toothpastes, other oral cavity compositions, alcoholic beverages such as vodka, wine, beer, liquor, and sake, etc., natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, frozen beef, frozen pork, frozen goat, frozen lamb, frozen mutton, frozen poultry like frozen chicken, frozen duck and frozen turkey, frozen venison, frozen fish, frozen crustaceans like frozen crab and frozen lobster, frozen molluscs like frozen clams, frozen oysters, frozen scallops, and frozen mussels, frozen shrimps, frozen octopus, frozen squid, fresh beef, fresh pork, fresh goat, fresh lamb, fresh mutton, fresh poultry like fresh chicken, fresh duck and fresh turkey, fresh venison, fresh fish, fresh crustaceans like fresh crab and fresh lobster, fresh molluscs like fresh clams, fresh oysters, fresh scallops, and fresh mussels, fresh shrimps, fresh octopus, fresh squid, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, medicinal products, lipsticks, and many others. In principle it can have unlimited applications.

During the manufacturing of products such as foodstuffs, drinks, pharmaceuticals, cosmetics, table top products, and chewing gum, the conventional methods such as mixing, kneading, dissolution, pickling, permeation, percolation, sprinkling, atomizing, infusing and other methods may be used.

Moreover, the highly purified target steviol glycoside(s) steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 obtained in this invention may be used in dry or liquid forms.

The highly purified target steviol glycoside can be added before or after heat treatment of food products. The amount of the highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 depends on the purpose of usage. As discussed above, it can be added alone or in combination with other compounds.

The present invention is also directed to sweetness enhancement in food and beverages using steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 as a sweetness enhancer, wherein steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 is present in a concentration at or below their respective sweetness recognition thresholds.

The present invention is also directed to flavor stabilization of food and beverages using steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 as a flavor stabilizer, wherein steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 is present in a concentration that allows for flavor stabilization of products.

The present invention is also directed to modification (including enhancing or suppressing) of flavor and/or taste profile of food and beverages using steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 as a flavoring with modifying properties (FMP), wherein steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 is present in a concentration that allows for modification (including enhancing or suppressing) of flavor and/or taste profile of products.

The present invention is also directed to foaming suppression of food and beverages using steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 as a foaming suppressor, wherein steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 is present in a concentration that allows for foaming suppression of products.

The present invention is also directed to solubility enhancement of insoluble material in food and beverages using steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 as a solubility enhancing agent, wherein steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 is present in a concentration that allows for solubility enhancement of insoluble material in products.

As used herein, the term “sweetness enhancer” refers to a compound or a mixture of compounds capable of enhancing or intensifying the perception of sweet taste in food and beverage products. The term “sweetness enhancer” is synonymous with the terms “sweet taste potentiator,” “sweetness potentiator,” “sweetness amplifier,” and “sweetness intensifier.”

As used herein, the term “flavor stabilizer” refers to a compound or a mixture of compounds capable of stabilizing the flavor in food and beverage products. It is contemplated that a flavor stabilizer can be used alone, or in combination with other flavor stabilizers.

As used herein, the term “flavoring with modifying properties (FMP)” refers to a compound or a mixture of compounds that enhance, subdue or otherwise affect the taste and/or flavor profile without themselves being sweeteners or flavorings. The Flavor and Extracts Manufacturing Association (FEMA) has developed a protocol published in the November 2013 Edition of Food Technology. It is contemplated that a flavoring with modifying properties (FMP) can be used alone, or in combination with other flavorings.

The term “sweetness recognition threshold concentration,” as generally used herein, is the lowest known concentration of a sweet compound that is perceivable by the human sense of taste, typically around 1.0% sucrose equivalence (1.0% SE). Generally, the sweetness enhancers may enhance or potentiate the sweet taste of sweeteners without providing any noticeable sweet taste by themselves when present at or below the sweetness recognition threshold concentration of a given sweetness enhancer; however, the sweetness enhancers may themselves provide sweet taste at concentrations above their sweetness recognition threshold concentration. The sweetness recognition threshold concentration is specific for a particular enhancer and can vary based on the beverage matrix. The sweetness recognition threshold concentration can be easily determined by taste testing increasing concentrations of a given enhancer until greater than 1.0% sucrose equivalence in a given beverage matrix is detected. The concentration that provides about 1.0% sucrose equivalence is considered the sweetness recognition threshold.

In some embodiments, sweetener is present in the beverage in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

In a particular embodiment, the sweetener is present in the beverage in an amount from about 0.0001% by weight to about 10% by weight, such as for example, from about 0.0001% by weight to about 0.0005% by weight, from about 0.0005% by weight to about 0.001% by weight, from about 0.001% by weight to about 0.005% by weight, from about 0.005% by weight to about 0.01% by weight, from about 0.01% by weight to about 0.05% by weight, from about 0.05% by weight to about 0.1% by weight, from about 0.1% by weight to about 0.5% by weight, from about 0.5% by weight to about 1% by weight, from about 1% by weight to about 2% by weight, from about 2% by weight to about 3% by weight, from about 3% by weight to about 4% by weight, from about 4% by weight to about 5% by weight, from about 5% by weight to about 6% by weight, from about 6% by weight to about 7% by weight, from about 7% by weight to about 8% by weight, from about 8% by weight to about 9% by weight, or from about 9% by weight to about 10% by weight. In a particular embodiment, the sweetener is present in the beverage in an amount from about 0.5% by weight to about 10% by weight. In another particular embodiment, the sweetener is present in the beverage in an amount from about 2% by weight to about 8% by weight.

In one embodiment, the sweetener is a traditional caloric sweetener. Suitable sweeteners include, but are not limited to, sucrose, fructose, glucose, high fructose corn syrup and high fructose starch syrup.

In another embodiment, the sweetener is erythritol.

In still another embodiment, the sweetener is a rare sugar. Suitable rare sugars include, but are not limited to, D-allose, D-psicose, D-ribose, D-tagatose, L-glucose, L-fucose, L-arabinose, D-turanose, D-leucrose, 5-ketofructose and combinations thereof.

It is contemplated that a sweetener can be used alone, or in combination with other sweeteners.

In one embodiment, the rare sugar is D-allose. In a more particular embodiment, D-allose is present in the beverage in an amount of about 0.5% to about 10% by weight, such as, for example, from about 2% to about 8%.

In another embodiment, the rare sugar is D-psicose. In a more particular embodiment, D-psicose is present in the beverage in an amount of about 0.5% to about 10% by weight, such as, for example, from about 2% to about 8%.

In still another embodiment, the rare sugar is D-ribose. In a more particular embodiment, D-ribose is present in the beverage in an amount of about 0.5% to about 10% by weight, such as, for example, from about 2% to about 8%.

In yet another embodiment, the rare sugar is D-tagatose. In a more particular embodiment, D-tagatose is present in the beverage in an amount of about 0.5% to about 10% by weight, such as, for example, from about 2% to about 8%.

In a further embodiment, the rare sugar is L-glucose. In a more particular embodiment, L-glucose is present in the beverage in an amount of about 0.5% to about 10% by weight, such as, for example, from about 2% to about 8%.

In one embodiment, the rare sugar is L-fucose. In a more particular embodiment, L-fucose is present in the beverage in an amount of about 0.5% to about 10% by weight, such as, for example, from about 2% to about 8%.

In another embodiment, the rare sugar is L-arabinose. In a more particular embodiment, L-arabinose is present in the beverage in an amount of about 0.5% to about 10% by weight, such as, for example, from about 2% to about 8%.

In still another embodiment, the rare sugar is D-turanose. In a more particular embodiment, D-turanose is present in the beverage in an amount of about 0.5% to about 10% by weight, such as, for example, from about 2% to about 8%.

In yet another embodiment, the rare sugar is D-leucrose. In a more particular embodiment, D-leucrose is present in the beverage in an amount of about 0.5% to about 10% by weight, such as, for example, from about 2% to about 8%.

In a further embodiment, the rare sugar is 5-ketofructose. In a more particular embodiment, 5-ketofructose is present in the beverage in an amount of about 0.5% to about 10% by weight, such as, for example, from about 2% to about 8%.

The addition of the sweetness enhancer at a concentration at or below its sweetness recognition threshold increases the detected sucrose equivalence of the beverage comprising the sweetener and the sweetness enhancer compared to a corresponding beverage in the absence of the sweetness enhancer. Moreover, sweetness can be increased by an amount more than the detectable sweetness of a solution containing the same concentration of the at least one sweetness enhancer in the absence of any sweetener.

Accordingly, the present invention also provides a method for enhancing the sweetness of a food or beverage comprising a sweetener comprising providing a food or beverage comprising a sweetener and adding a sweetness enhancer selected from steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 or a combination thereof, wherein steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 are present in a concentration at or below their sweetness recognition thresholds.

Accordingly, the present invention also provides a method for stabilizing the flavor of a food or beverage comprising providing a food or beverage and adding a flavor stabilizer selected from steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 or a combination thereof, wherein steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 are present in a concentration that allows for improved flavor stability compared to a control product that does not contain the flavor stabilizer.

Accordingly, the present invention also provides a method for modification (including enhancing or suppressing) of flavor and/or taste profile of a food or beverage comprising providing a food or beverage and adding a flavoring with modifying properties (FMP) selected from steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 or a combination thereof, wherein steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 are present in a concentration that allows for modification (including enhancing or suppressing) of flavor and/or taste profile.

In one embodiment, the present invention also provides a method for adding steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 in a concentration at or below the sweetness recognition threshold to a food or beverage containing a sweetener to increase the detected sucrose equivalence from about 1.0% to about 5.0%, such as, for example, about 1.0%, about 1.5%, about 2.0%, about 2.5%, about 3.0%, about 3.5%, about 4.0%, about 4.5% or about 5.0%.

In another embodiment, the present invention also provides a method for adding steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4 in a concentration that allows for modification (including enhancing or suppressing) of flavor and/or taste profile to a food or beverage to modify (including enhancing or suppressing) the flavor and/or taste profile.

This invention provides rebaudioside D9 with the following formula:

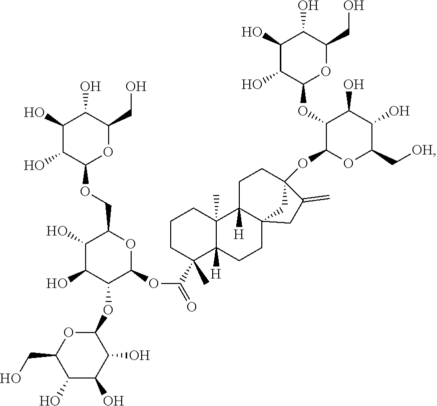

including salts thereof, or combinations thereof.

Furthermore, this disclosure provides a method for producing rebaudioside D9, comprising the steps of providing a starting composition comprising an organic compound with at least one carbon atom; providing an enzyme preparation or microorganism containing at least one enzyme selected from the group consisting of steviol biosynthesis enzymes and NDP-glucosyltransferases and optionally NDP-glucose recycling enzymes; contacting the enzyme preparation or microorganism with a medium containing the starting composition to produce a medium comprising rebaudioside D9.

Also, this disclosure provides a method for producing rebaudioside D9, comprising the steps of providing a starting composition comprising an organic compound with at least one carbon atom; providing a biocatalyst comprising at least one enzyme selected from the group consisting of steviol biosynthesis enzymes and NDP-glucosyltransferases and optionally NDP-glucose recycling enzymes; contacting the biocatalyst with a medium containing the starting composition to produce a medium comprising rebaudioside D9.

Optionally, the method above further comprises the step of separating rebaudioside D9 from the medium to provide a highly purified composition of rebaudioside D9.

In the methods above, the starting composition is selected from the group consisting of steviol, steviolmonoside, steviolmonoside A, steviolbioside, steviolbioside A, steviolbioside G, rubusoside, stevioside, stevioside A, stevioside K, stevioside L, rebaudioside E, rebaudioside E8, rebaudioside E12, other steviol glycosides, polyols, carbohydrates, and combinations thereof.

The microorganism is selected from the group consisting of E. coli, Saccharomyces sp., Aspergillus sp., Pichia sp., Bacillus sp., and Yarrowia sp.

The biocatalyst is an enzyme, or a cell comprising one or more enzyme, capable of converting the starting composition to rebaudioside D9.

The enzyme is selected from the group consisting of a mevalonate (MVA) pathway enzyme, a 2-C-methyl-D-erythritol-4-phosphate pathway (MEP/DOXP) enzyme, geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase, UGT74G1, UGT85C2, UGTS12, UGT76G1, EUGT11, UGT91D2 or mutant variant thereof having >85% amino-acid sequence identity, >86% amino-acid sequence identity, >87% amino-acid sequence identity, >88% amino-acid sequence identity, >89% amino-acid sequence identity, >90% amino-acid sequence identity, >91% amino-acid sequence identity, >92% amino-acid sequence identity, >93% amino-acid sequence identity, >94% amino-acid sequence identity, >95% amino-acid sequence identity, >96% amino-acid sequence identity, >97% amino-acid sequence identity, >98% amino-acid sequence identity, >99% amino-acid sequence identity.

The content of rebaudioside D9 in the highly purified composition of rebaudioside D9 is greater than about 80%, greater than about 90%, greater than about 91%, greater than about 92%, greater than about 93%, greater than about 94%, greater than about 95%, greater than about 96%, greater than about 97%, greater than about 98% or greater than about 99% by weight on a dried basis.

This invention also provides a consumable product comprising rebaudioside D9, wherein the product is selected from the group consisting of foods, beverages, pharmaceutical compositions, tobacco products, nutraceutical compositions, oral hygiene compositions, and cosmetic compositions.

Also, the consumable product that this invention provides is selected from the group consisting of foodstuffs, beverages, pharmaceutical compositions, cosmetics, chewing gums, table top products, cereals, dairy products, toothpastes, other oral cavity compositions, alcoholic beverages such as vodka, wine, beer, liquor, and sake, etc., natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, frozen beef, frozen pork, frozen goat, frozen lamb, frozen mutton, frozen poultry like frozen chicken, frozen duck and frozen turkey, frozen venison, frozen fish, frozen crustaceans like frozen crab and frozen lobster, frozen molluscs like frozen clams, frozen oysters, frozen scallops, and frozen mussels, frozen shrimps, frozen octopus, frozen squid, fresh beef, fresh pork, fresh goat, fresh lamb, fresh mutton, fresh poultry like fresh chicken, fresh duck and fresh turkey, fresh venison, fresh fish, fresh crustaceans like fresh crab and fresh lobster, fresh molluscs like fresh clams, fresh oysters, fresh scallops, and fresh mussels, fresh shrimps, fresh octopus, fresh squid, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, medicinal products, lipsticks, and many others.

The consumable product that this invention provides further comprises at least one additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, caffeine, flavorants and flavoring ingredients, flavorings with modifying properties (FMP), astringent compounds, proteins or protein hydrolysates, surfactants, emulsifiers, flavonoids, alcohols, polymers and combinations thereof.

The consumable product that this invention provides further comprises at least one functional ingredient selected from the group consisting of saponins, antioxidants, dietary fiber sources, fatty acids, vitamins, glucosamine, minerals, preservatives, hydration agents, probiotics, prebiotics, postbiotics, weight management agents, osteoporosis management agents, phytoestrogens, long chain primary aliphatic saturated alcohols, phytosterols and combinations thereof.

The consumable product that this invention provides further comprises a compound selected from the group consisting of dulcoside A, dulcoside B, dulcoside C, dulcoside D, rebaudioside 1a, rebaudioside 1b, rebaudioside 1c, rebaudioside 1d, rebaudioside 1e, rebaudioside 1f, rebaudioside 1g, rebaudioside 1h, rebaudioside 1i, rebaudioside 1j, rebaudioside 1k, rebaudioside 1l, rebaudioside 1m, rebaudioside 1n, rebaudioside 10, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f, rebaudioside 2g, rebaudioside 2h, rebaudioside 2i, rebaudioside 2j, rebaudioside 2k, rebaudioside 21, rebaudioside 2m, rebaudioside 2n, rebaudioside 2o, rebaudioside 2p, rebaudioside 2q, rebaudioside 2r, rebaudioside 2s, rebaudioside A, rebaudioside AIG, rebaudioside A2, rebaudioside A3, rebaudioside A4, rebaudioside AM, rebaudioside B, rebaudioside B2, rebaudioside C, rebaudioside C2, rebaudioside C3, rebaudioside C4, rebaudioside C5, rebaudioside C6, rebaudioside C7, rebaudioside D, rebaudioside D2, rebaudioside D3, rebaudioside D4, rebaudioside D5, rebaudioside D6, rebaudioside D7, rebaudioside D8, rebaudioside D9, rebaudioside D10, rebaudioside D11, rebaudioside D12, rebaudioside D13, rebaudioside E, rebaudioside E2, rebaudioside E3, rebaudioside E4, rebaudioside E5, rebaudioside E6, rebaudioside E7, rebaudioside E8, rebaudioside E9, rebaudioside E10, rebaudioside E11, rebaudioside E12, rebaudioside E13, rebaudioside F, rebaudioside F1, rebaudioside F2, rebaudioside F3, rebaudioside G, rebaudioside H, rebaudioside H1, rebaudioside H2, rebaudioside H3, rebaudioside H4, rebaudioside H5, rebaudioside H6, rebaudioside H7, rebaudioside I, rebaudioside I2, rebaudioside I3, rebaudioside IX, rebaudioside IXa, rebaudioside IXb, rebaudioside IXc, rebaudioside IXd, rebaudioside J, rebaudioside K, rebaudioside K2, rebaudioside KA, rebaudioside L, rebaudioside L1, rebaudioside M, rebaudioside M2, rebaudioside M3, rebaudioside M4, rebaudioside M5, rebaudioside N, rebaudioside N2, rebaudioside N3, rebaudioside N4, rebaudioside N5, rebaudioside N6, rebaudioside N7, rebaudioside O, rebaudioside O2, rebaudioside O3, rebaudioside O4, rebaudioside O5, rebaudioside O6, rebaudioside O7, rebaudioside Q, rebaudioside Q2, rebaudioside Q3, rebaudioside R, rebaudioside R1, rebaudioside S, rebaudioside T, rebaudioside T1, rebaudioside U, rebaudioside U2, rebaudioside U3, rebaudioside V, rebaudioside V2, rebaudioside VIII, rebaudioside VIIIa, rebaudioside VIIIb, rebaudioside W, rebaudioside W2, rebaudioside W3, rebaudioside WB1, rebaudioside WB2, rebaudioside Y, rebaudioside Z1, rebaudioside Z2, rubusoside, steviolbioside, steviolbioside A, steviolbioside B, steviolbioside C, steviolbioside D, steviolbioside E, steviolbioside F, steviolbioside G, steviolmonoside, steviolmonoside A, stevioside, stevioside A, stevioside B, stevioside C, stevioside D, stevioside E, stevioside E2, stevioside F, stevioside G, stevioside H, stevioside I, stevioside J, stevioside K, stevioside L, stevioside M, SvG7, NSF-02, Mogroside V, siratose, Luo Han Guo, allulose, D-allose, D-tagatose, erythritol, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, siamenoside, sucralose, potassium acesulfame, aspartame, alitame, saccharin, cyclamate, neotame, dulcin, suosan advantame, gymnemic acid, hodulcin, ziziphin, lactisole, glutamate, aspartic acid, glycine, alanine, threonine, proline, serine, lysine, tryptophan, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols, sugar alcohols, L-sugars, L-sorbose, L-arabinose, trehalose, galactose, rhamnose, various cyclodextrins, cyclic oligosaccharides, various types of maltodextrins, dextran, sucrose, glucose, ribulose, fructose, threose, xylose, lyxose, altrose, mannose, idose, lactose, maltose, invert sugar, isotrehalose, neotrehalose, isomaltulose, erythrose, deoxyribose, gulose, talose, erythrulose, xylulose, cellobiose, amylopectin, glucosamine, mannosamine, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, isomerized liquid sugars such as high fructose corn syrups, coupling sugars, soybean oligosaccharides, D-psicose, D-ribose, L-glucose, L-fucose, D-turanose, D-leucrose, 5-ketofructose and combinations thereof.

This invention also provides a method for enhancing the sweetness of a beverage or food product, comprising a sweetener providing a beverage or food product comprising a sweetener; and adding a sweetness enhancer comprising rebaudioside D9, wherein rebaudioside D9 is present in a concentration at or below the sweetness recognition threshold or in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

This invention also provides a method for stabilizing the flavor of a beverage or food product, comprising providing a beverage or food product; and adding a flavor stabilizer comprising rebaudioside D9, wherein rebaudioside D9 is present in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

This invention also provides a method for modification (including enhancing or suppressing) of flavor and/or taste profile of a beverage or food product, comprising providing a beverage or food product; and adding a flavoring with modifying properties (FMP) comprising rebaudioside D9, wherein rebaudioside D9 is present in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

This invention also provides a method for suppressing foaming of a beverage or food product, comprising providing a beverage or food product; and adding a foam suppressor comprising rebaudioside D9, wherein rebaudioside D9 is present in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

This invention also provides a method for enhancing the solubility of insoluble material in a beverage or food product, comprising providing a beverage or food product containing insoluble material; and adding a solubility enhancing agent comprising rebaudioside D9, wherein rebaudioside D9 is present in an amount from about 0.0001% to about 12% by weight, such as, for example, about 0.0001% by weight, about 0.0005% by weight, about 0.001% by weight, about 0.005% by weight, about 0.01% by weight, about 0.05% by weight, about 0.1% by weight, about 0.5% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

The following examples illustrate preferred embodiments of the invention for the preparation of highly purified target steviol glycoside(s), particularly steviolmonoside, steviolmonoside A, steviolbioside, steviolbioside A, steviolbioside B, steviolbioside F, steviolbioside G, rubusoside, stevioside, stevioside A, stevioside B, stevioside C, stevioside I, stevioside J, stevioside K, stevioside L, rebaudioside E, rebaudioside E2, rebaudioside E3, rebaudioside E8, rebaudioside E9, rebaudioside E10, rebaudioside E11, rebaudioside E12, rebaudioside D9, rebaudioside D10, rebaudioside D11, rebaudioside D12, rebaudioside AM and/or rebaudioside M4. It will be understood that the invention is not limited to the materials, proportions, conditions and procedures set forth in the examples, which are only illustrative.

### EXAMPLES

### Example 1

### Protein Sequences of Engineered Enzymes Used in the Biocatalytic Process

>SuSy_At, variant PM1-54-2-E05 (engineered
sucrose synthase; source of WT gene:
Arabidopsis thaliana)
SEQ ID 1:
MANAERMITRVHSQRERLNETLVSERNEVLALLSRVEAKGKGILQQNQI
IAEFEALPEQTRKKLEGGPFFDLLKSTQEAIVLPPWVALAVRPRPGVWE
YLRVNLHALVVEELQPAEFLHFKEELVDGVKNGNFTLELDFEPFNASIP
RPTLHKYIGNGVDFLNRHLSAKLFHDKESLLPLLDFLRLHSHQGKNLML
SEKIQNLNTLQHTLRKAEEYLAELKSETLYEEFEAKFEEIGLERGWGDN
AERVLDMIRLLLDLLEAPDPSTLETFLGRVPMVFNVVILSPHGYFAQDN
VLGYPDTGGQVVYILDQVRALEIEMLQRIKQQGLNIKPRILILTRLLPD
AVGTTCGERLERVYDSEYCDILRVPFRTEKGIVRKWISRFEVWPYLETY
TEDAAVELSKELNGKPDLIIGNYSDGNLVASLLAHKLGVTQCTIAHALE
KTKYPDSDIYWKKLDDKYHFSCQFTADIFAMNHTDFIITSTFQEIAGSK
ETVGQYESHTAFTLPGLYRVVHGIDVFDPKFNIVSPGADMSIYFPYTEE
KRRLTKFHSEIEELLYSDVFNDEHLCVLKDKKKPILFTMARLDRVKNLS
GLVEWYGKNTRLRELVNLVVVGGDRRKESKDNEEKAEMKKMYDLIEEYK
LNGQFRWISSQMDRVRNGELYRYICDTKGAFVQPALYEAFGLTVVEAMT
CGLPTFATCKGGPAEIIVHGKSGFHIDPYHGDQAADLLADFFTKCKEDP
SHWDEISKGGLQRIEEKYTWQIYSQRLLTLTGVYGFWKHVSNLDRLEHR
RYLEMFYALKYRPLAQAVPLAQDD

>UGT74G1 (glucosyltransferase; source of WT gene:
Stevia rebaudiana)
SEQ ID 2:
MAEQQKIKKSPHVLLIPFPLQGHINPFIQFGKRLISKGVKTTLVTTIHT
LNSTLNHSNTTTTSIEIQAISDGCDEGGFMSAGESYLETFKQVGSKSLA
DLIKKLQSEGTTIDAIIYDSMTEWVLDVAIEFGIDGGSFFTQACVVNSL
YYHVHKGLISLPLGETVSVPGFPVLQRWETPLILQNHEQIQSPWSQMLF
GQFANIDQARWVFTNSFYKLEEEVIEWTRKIWNLKVIGPTLPSMYLDKR
LDDDKDNGFNLYKANHHECMNWLDDKPKESVVYVAFGSLVKHGPEQVEE
ITRALIDSDVNFLWVIKHKEEGKLPENLSEVIKTGKGLIVAWCKQLDVL
AHESVGCFVTHCGFNSTLEAISLGVPVVAMPQFSDQTTNAKLLDEILGV
GVRVKADENGIVRRGNLASCIKMIMEEERGVIIRKNAVKWKDLAKVAVH
EGGSSDNDIVEFVSELIKA

>UGT85C2 (glucosyltransferase; source of WT gene:
Stevia rebaudiana)
SEQ ID 3:
MDAMATTEKKPHVIFIPFPAQSHIKAMLKLAQLLHHKGLQITFVNTDFI
HNQFLESSGPHCLDGAPGFRFETIPDGVSHSPEASIPIRESLLRSIETN
FLDRFIDLVTKLPDPPTCIISDGFLSVFTIDAAKKLGIPVMMYWTLAAC
GFMGFYHIHSLIEKGFAPLKDASYLTNGYLDTVIDWVPGMEGIRLKDFP
LDWSTDLNDKVLMFTTEAPQRSHKVSHHIFHTFDELEPSIIKTLSLRYN
HIYTIGPLQLLLDQIPEEKKQTGITSLHGYSLVKEEPECFQWLQSKEPN
SVVYVNFGSTTVMSLEDMTEFGWGLANSNHYFLWIIRSNLVIGFNAVLP
PELEEHIKKRGFIASWCSQEKVLKHPSVGGFLTHCGWGSTIESLSAGVP
MICWPYSWDQLTNCRYICKEWEVGLEMGTKVKRDEVKRLVQELMGEGGH
KMRNKAKDWKEKARIAIAPNGSSSLNIDKMVKEITVLARN

>UGTS12 variant 0234 (engineered
glucosyltransferase; source of WT gene:
Solanum lycopersicum)
SEQ ID 4:
MATNLRVLMFPWLAYGHISPFLNIAKQLADRGFLIYLCSTRINLESIIK
KIPEKYADSIHLIELQLPELPELPPHYHTTNGLPPHLNPTLHKALKMSK
PNFSRILQNLKPDLLIYDVLQPWAEHVANEQGIPAGKLLVSCAAVESYF
FSFRKNPGVEFPFPAIHLPEVEKVKIREILAKEPEEGGRLDEGNKQMML
MCTSRTIEAKYIDYCTELCNWKVVPVGPPFQDLITNDADNKELIDWLGT
KPENSTVFVSFGSEYFLSKEDMEEIAFALEASNVNFIWVVRFPKGEERN
LEDALPEGFLERIGERGRVLDKFAPQPRILNHPSTGGFISHCGWNSVME
SIDFGVPIIAMPIHNDQPINAKLMVELGVAVEIVRDDDGKIHRGEIAEA
LKSVVTGETGEILRAKVREISKNLKSIRDEEMDAVAEELIQLCRNSNKS
K

>UGT76G1 variant 0042 (engineered
glucosyltransferase; source of WT gene:
Stevia rebaudiana)
SEQ ID 5:
MENKTETTVRRRRRIILFPVPFQGHINPILQLANVLYSKGFAITILHT
NFNKPKTSNYPHFTFRFILDNDPQDERISNLPTHGPLAGMRIPIINEH
GADELRRELELLMLASEEDEEVSCLITDALWYFAQDVADSLNLRRLVL
MTSSLFNFHAHVSLPQFDELGYLDPDDKTRLEEQASGFPMLKVKDIKS
AYSNWQIGKEILGKMIKQTKASSGVIWNSFKELEESELETVIREIPAP
SFLIPLPKHLTASSSSLLDHDRTVFEWLDQQAPSSVLYVSFGSTSEVD
EKDFLEIARGLVDSGQSFLWVVRPGFVKGSTWVEPLPDGFLGERGKIV
KWVPQQEVLAHPAIGAFWTHSGWNSTLESVCEGVPMIFSSFGGDQPLN
ARYMSDVLRVGVYLENGWERGEVVNAIRRVMVDEEGEYIRQNARVLKQ
KADVSLMKGGSSYESLESLVSYISSL

>EUGT11 (glucosyltransferase; source of WT gene:
Oryza sativa Japonica)
SEQ ID 6:
MDSGYSSSYAAAAGMHVVICPWLAFGHLLPCLDLAQRLASRGHRVSFVS
TPRNISRLPPVRPALAPLVAFVALPLPRVEGLPDGAESTNDVPHDRPDM
VELHRRAFDGLAAPFSEFLGTACADWVIVDVFHHWAAAAALEHKVPCAM
MLLGSAHMIASIADRRLERAETESPAAAGQGRPAAAPTFEVARMKLIRT
KGSSGMSLAERFSLTLSRSSLVVGRSCVEFEPETVPLLSTLRGKPITFL
GLMPPLHEGRREDGEDATVRWLDAQPAKSVVYVALGSEVPLGVEKVHEL
ALGLELAGTRFLWALRKPTGVSDADLLPAGFEERTRGRGVVATRWVPQM
SILAHAAVGAFLTHCGWNSTIEGLMFGHPLIMLPIFGDQGPNARLIEAK
NAGLQVARNDGDGSFDREGVAAAIRAVAVEEESSKVFQAKAKKLQEIVA
DMACHERYIDGFIQQLRSYKD

>UGT91D2 (glucosyltransferase; source of WT gene:
Stevia rebaudiana)
SEQ ID 7:
MATSDSIVDDRKQLHVATFPWLAFGHILPYLQLSKLIAEKGHKVSFLST
TRNIQRLSSHISPLINVVQLTLPRVQELPEDAEATTDVHPEDIPYLKKA
SDGLQPEVTRFLEQHSPDWIIYDYTHYWLPSIAASLGISRAHFSVTTPW
AIAYMGPSADAMINGSDGRTTVEDLTTPPKWFPFPTKVCWRKHDLARLV
PYKAPGISDGYRMGLVLKGSDCLLSKCYHEFGTQWLPLLETLHQVPVVP
VGLLPPEVPGDEKDETWVSIKKWLDGKQKGSVVYVALGSEVLVSQTEVV
ELALGLELSGLPFVWAYRKPKGPAKSDSVELPDGFVERTRDRGLVWTSW
APQLRILSHESVCGELTHCGSGSIVEGLMFGHPLIMLPIFGDQPLNARL
LEDKQVGIEIPRNEEDGCLTKESVARSLRSVVVEKEGEIYKANARELSK
IYNDTKVEKEYVSQFVDYLEKNTRAVAIDHES

### Example 2

### Expression and Formulation of SuSy_at Variant of SEQ ID 1

The gene coding for the SuSy_At variant of SEQ ID 1 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/1) at 37° C. Expression of the genes was induced at logarithmic phase by IPTG (0.2 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with distilled water, resulting in an enzymatic active preparation.

For enzymatic active preparations of SuSy_At, activity in Units is defined as follows: 1 mU of SuSy_At turns over 1 nmol of sucrose into fructose in 1 minute. Reaction conditions for the assay are 30° C., 50 mM potassium phosphate buffer pH 7.0, 400 mM sucrose at to, 3 mM MgCl₂, and 15 mM uridine diphosphate (UDP).

### Example 3

### Expression and Formulation of UGTS12 Variant of SEQ ID 4

The gene coding for the UGTS12 variant of SEQ ID 4 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/1) at 37° C. Expression of the genes was induced at logarithmic phase by IPTG (0.1 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with 1 M sucrose solution, resulting in an enzymatic active preparation.

For enzymatic active preparations of UGTS12, activity in Units is defined as follows: 1 mU of UGTS12 turns over 1 nmol of rebaudioside A (Reb A) into rebaudioside D (Reb D) in 1 minute. Reaction conditions for the assay are 30° C., 50 mM potassium phosphate buffer pH 7.0, 10 mM Reb A at to, 500 mM sucrose, 3 mM MgCl₂, 0.25 mM uridine diphosphate (UDP) and 3 U/mL of SuSy_At.

### Example 4

### Expression and Formulation of UGT76G1 Variant of SEQ ID 5

The gene coding for the UGT76G1 variant of SEQ ID 5 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/1) at 37° C. Expression of the genes was induced at logarithmic phase by IPTG (0.1 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with 1 M sucrose solution, resulting in an enzymatic active preparation.

For enzymatic active preparations of UGT76G1, activity in Units is defined as follows: 1 mU of UGT76G1 turns over 1 nmol of rebaudioside D (Reb D) into rebaudioside M(Reb M) in 1 minute. Reaction conditions for the assay are 30° C., 50 mM potassium phosphate buffer pH 7.0, 10 mM Reb D at to, 500 mM sucrose, 3 mM MgCl₂, 0.25 mM uridine diphosphate (UDP) and 3 U/mL of SuSy_At.

### Example 5

### Synthesis of Rebaudioside D9 and Rebaudioside M4 in a One-Pot Reaction, Adding UGTS12, SuSy_at and UGT76G1 at the Same Time.

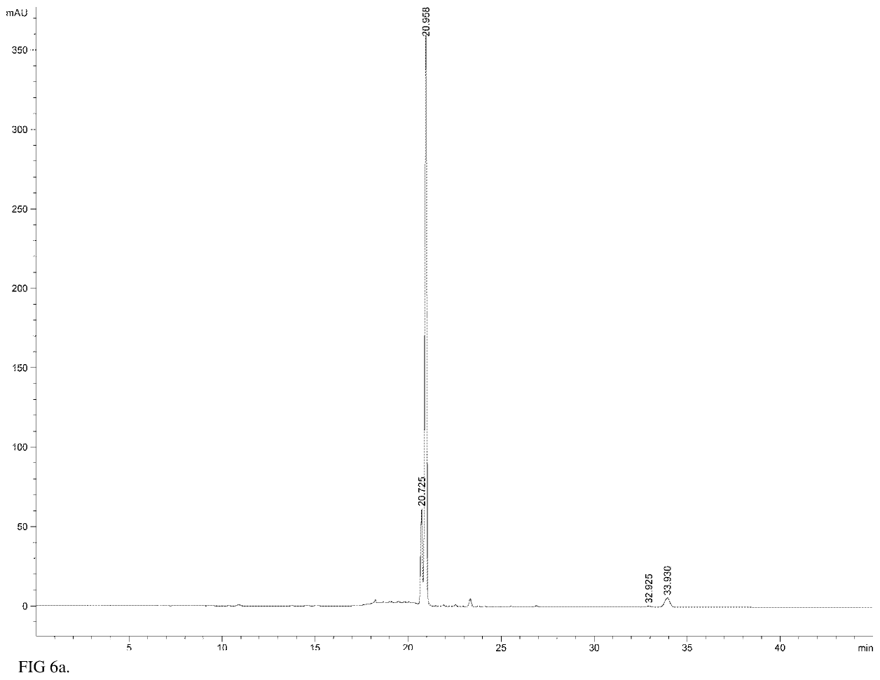

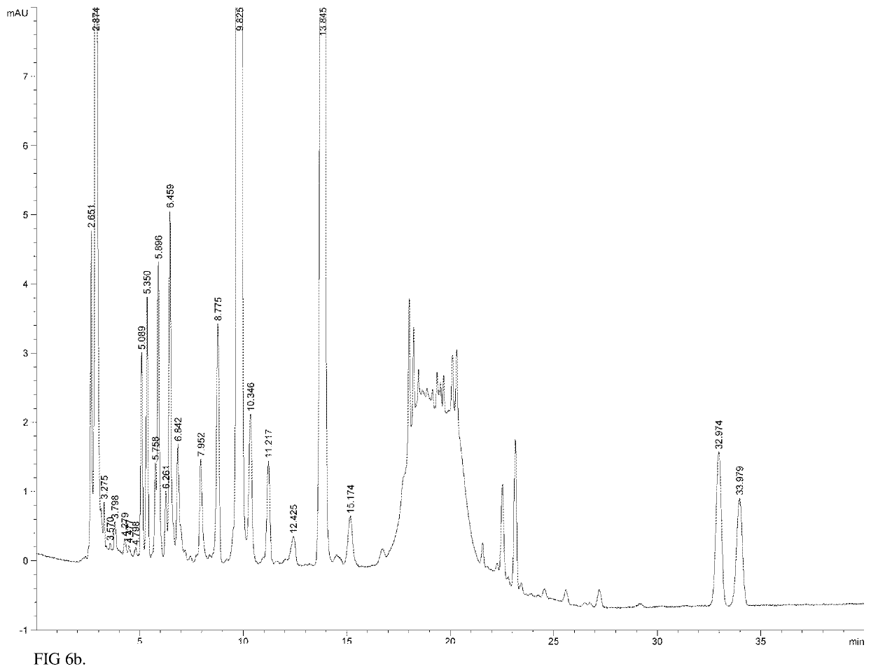

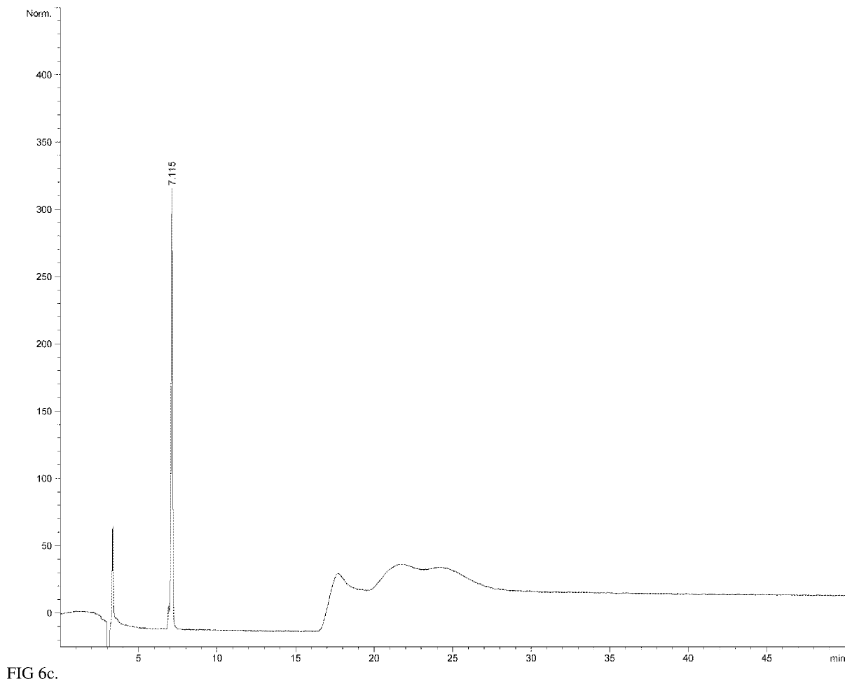

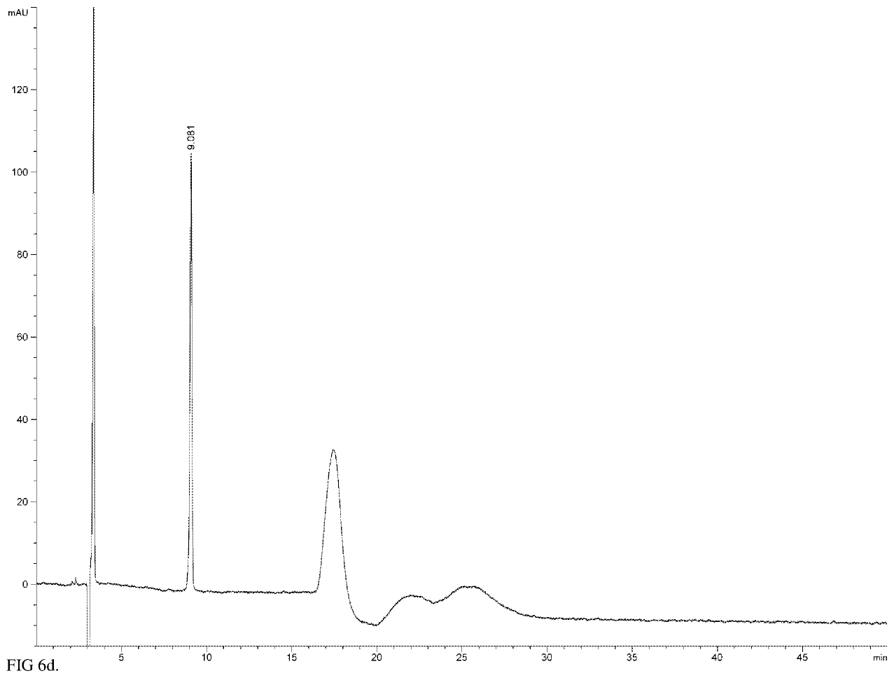

Rebaudioside D9, rebaudioside M4 and various steviol glycoside molecules were synthesized directly from stevioside (see FIG. 6a) in a one-pot reaction, utilizing the three enzymes (see EXAMPLES 1, 2, 3 and 4): UGTS12 (variant of SEQ ID 4), SuSy_At (variant of SEQ ID 1) and UGT76G1 (variant of SEQ ID 5).

The final reaction solution contained 348 U/L UGTS12, 1341 U/L SuSy_At, 10 U/L UGT76G1, 47 mM stevioside, 0.32 mM uridine diphosphate (UDP), 0.99 M sucrose, 3.9 mM MgCl₂ and potassium phosphate buffer (pH 6.6). First, 206 mL of distilled water were mixed with 0.24 g MgCl₂·6H₂O, 102 g sucrose, 9.8 mL of 1.5 M potassium phosphate buffer (pH 6.6) and 15 g stevioside. The final volume of the reaction mixture was adjusted to 300 mL.

After dissolving the components, the temperature was adjusted to 45° C. and UGTS12, SuSy_At, UGT76G1 and 39 mg UDP were added. The reaction mixture was incubated at 45° C. shaker for 24 hrs. Additional 39 mg UDP was added at 12 hours, 24 hours, and 36 hours. The content of reb D9, reb M4 and various steviol glycosides at the end of the reaction (48 hours) was analyzed by HPLC.

### Example 6

### HPLC Analysis

Reaction mixture samples were inactivated by adjusting the pH to pH5.5 using 17% H₃PO₄ and then boiled for 10 minutes. Resulting samples were filtered, the filtrates were diluted 10 times and used as samples for HPLC analysis. HPLC assay was carried out on Agilent HP 1200 HPLC system, comprised of a pump, a column thermostat, an auto sampler, a UV detector capable of background correction and a data acquisition system. Analytes were separated using Agilent Poroshell 120 SB—C18, 4.6 mm×150 mm, 2.7 μm at 40° C. The mobile phase consisted of two premixes:
premix 1 containing 75% 10 mM phosphate buffer (pH2.6) and 25% acetonitrile, and
premix 2 containing 68% 10 mM phosphate buffer (pH2.6) and 32% acetonitrile.

Elution gradient started with premix 1, changed to premix 2 to 50% at 12.5 minute, changed to premix 2 to 100% at 13 minutes. Total run time was 45 minutes. The column temperature was maintained at 40° C. The injection volume was 5 μL. Steviol glycoside species were detected by UV at 210 nm.

Table 1 shows for each time point the conversion of stevioside into identified steviol glycoside species (area percentage). The chromatograms of the starting material stevioside and the reaction mixture at 48 hours are shown in FIG. 6a and FIG. 6b respectively. Those with skill in the art will appreciate that retention times can occasionally vary with changes in solvent and/or equipment.

TABLE 1
Synthesis of reb D9 (rt 5.896) and reb M4 (rt 8.775)
and various steviol glycosides from stevioside

% conversion from stevioside

| | reaction time 0 | reaction time 48 |
|---|---|---|
| Peak | hr | hr |
| rt 2.651 | 0 | 0.83 |
| rt 2.874 | 0 | 6.79 |
| rt 3.275 | 0 | 0.15 |
| rt 3.570 | 0 | 0.05 |
| rt 3.798 | 0 | 0.18 |
| rt 4.279 | 0 | 0.08 |
| rt 4.477 | 0 | 0.05 |
| rt 4.798 | 0 | 0.04 |
| rt 5.089 | 0 | 0.68 |
| rt 5.350 | 0 | 0.86 |
| rt 5.758 | 0 | 0.28 |
| rt 5.896 | 0 | 1.06 |
| rt 6.261 | 0 | 0.22 |
| rt 6.459 | 0 | 1.37 |
| rt 6.842 | 0 | 0.57 |
| rt 7.952 | 0 | 0.5 |
| rt 8.775 | 0 | 1.07 |
| reb AM | 0 | 65.7 |
| rt 10.346 | 0 | 0.83 |
| rt 11.217 | 0 | 0.54 |
| rt 12.425 | 0 | 0.2 |
| reb M | 0 | 14.97 |
| rt 15.174 | 0 | 0.4 |
| reb A | 12.97 | 0 |
| stevioside | 82.83 | 0 |
| reb B | 0.38 | 1.47 |
| steviolbioside | 3.82 | 1.11 |

### Example 7

### Purification of Rebaudioside D9 and Rebaudioside M4

300 mL of the reaction mixture of EXAMPLE 5, (after 48 hrs), was inactivated by adjusting the pH to pH 5.5 with H₃PO₄ and then boiled for 10 minutes and filtered. The filtrate was loaded into a column containing 500 mL YWD03 (Cangzhou Yuanwei, China) resin pre-equilibrated with water. The resin was washed with 2.5 L water and the water effluent from this step was discarded.

The steviol glycosides were eluted from the YWD03 resin column with 2.5 L 70% v/v ethanol/water. The effluent from this step was collected and dried under vacuum at 60° C. to yield 20g of dried solid product.

The obtained dried solid was dissolved in 2:3:5 v:v:v water:1-propanol:ethyl acetate and loaded onto a column containing silica gel (300 mL) and eluted with the same solvent. The effluents were collected multiple fractions of 50 mL. Fractions containing Reb D9 were combined and dried by rotary evaporation until bulk solvent was removed. Other fractions containing Reb M4 were combined and dried by rotary evaporation until bulk solvent was removed.

The combined dried fractions were dissolved in water and subjected to further fractionation and separation by HPLC, using the conditions listed in Table 2 below. Reb D9 fractions from multiple HPLC separation runs were combined and freeze-dried. Reb M4 fractions from multiple HPLC separation runs were combined and freeze-dried.

TABLE 2
Conditions for preparative HPLC

| Column | Agilent Poroshell 120 SB-C18, | |
|---|---|---|
| | 4.6 mm × 150 mm, 2.7 μm | |
| Temperature | 40° | C. |
| Mobile Phase | Isocratic - Water 75% Acetonitrile 25% | |
| Flow rate | 0.5 | mL/min |
| Injection | 10 | μL |
| Stop time | 20 | mins |
| Autosampler | Ambient | |
| temperature | | |

| | |
|---|---|
| Detection | UV at 210 nm |

The purity of obtained rebaudioside D9 fraction was evaluated by LCMS method described in EXAMPLE 8. The chromatogram of purified rebaudioside D9 is shown in FIG. 6c. The purity of obtained rebaudioside M4 fraction was evaluated by LCMS method described in EXAMPLE 9. The chromatogram of purified rebaudioside M4 is shown in FIG. 6d.

### Example 8

### Structure Elucidation of Rebaudioside D9

NMR experiments were performed on a Bruker 800 MHz spectrometer, with the rebaudioside D9 sample, prepared according to EXAMPLE 7, dissolved in pyridine-d5. Along with signals from the sample, signals from pyridine-d5 at 8c 123.5, 135.5, 149.9 ppm and δ_{H} 7.19, 7.55, 8.71 ppm were observed. 1H-NMR and 13C-NMR spectra of rebaudioside D9 confirmed the excellent quality of the sample. HSQC shows the presence of an exo-methylene group in the sugar region with a long-range coupling to C-15, observable in the H,H—COSY. Correlation of the signals in the HSQC, HMBC and H,H—COSY reveal the presence of steviol glycoside with the following aglycone structure:

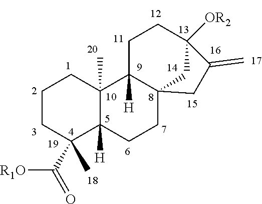

Correlation of HSQC and HMBC shows the presence five anomeric signals, marked as 1a, 1b, 1c, 1d, and 1e. The coupling constant of the anomeric protons of about 8 Hz and the NOE-correlations of the anomeric protons allow the identification of these five sugars as β-D-glucopyranosides.

Combined data from HSQC and HMBC reveal the sugar-sugar linkages and sugar-aglycone linkages. The assignment of the sugar sequence was further supported by TOCSY and NOESY.

Altogether, results from NMR experiments above were used to assign the chemical shifts of the protons and carbons of the structure of rebaudioside D9 (see Table 3).

TABLE 3
Chemical shifts of rebaudioside D9

| | | | | |
|---|---|---|---|---|
| | | | J [Hz]/ | HMBC |
| Position | δ_{C} [ppm] | δ_{H} [ppm] | (INT) | (H→C) |

Aglycone moiety

| | | | | | | |
|---|---|---|---|---|---|---|
| 1 | 40.59 | CH2 | 0.69 | m | | 2, 9, 10, 20 |
| | | | 1.70 | m | | |
| 2 | 20.00 | CH2 | 1.42 | m | | 1, 3, 10 |
| | | | 2.13 | m | | |
| 3 | 37.73 | CH2 | 1.05 | m | | 2, 4, 18, 19 |
| | | | 2.75 | m | | |
| 4 | 44.34 | C | — | — | | — |
| 5 | 57.39 | CH | 0.94 | m | | 4, 6, 7, 9, 10, 18, 19 |
| 6 | 22.05 | CH2 | 1.82 | m | | 5, 7, 8, 10 |
| | | | 2.08 | m | | |
| 7 | 41.61 | CH2 | 1.23 | m | | 5, 6, 8, 9, 14, 15 |
| | | | 1.31 | m | | |
| 8 | 42.64 | C | — | — | | — |
| 9 | 54.01 | CH | 0.84 | m | | 1, 5, 8, 10, 11, 12, |
| | | | | | | 14, 15, 20 |
| 10 | 39.67 | C | — | — | | —, |
| 11 | 20.59 | CH2 | 1.62 | m | | 8, 9, 10, 12, 13 |
| 12 | 37.40 | CH2 | 1.95 | m | | 11, 13, 16 |
| | | | 2.16 | m | | |
| 13 | 86.08 | C | — | | | — |
| 14 | 44.32 | CH2 | 1.75 | d | 11.6 | 7, 8, 9, 12, 13, 15, |
| | | | 2.51 | d | 11.6 | 16 |
| 15 | 47.97 | CH2 | 2.00 | d | 16.6 | 7, 8, 13, 16 |
| | | | 2.06 | d | 16.6 | |
| 16 | 154.54 | C | — | — | | — |
| 17 | 104.72 | CH2 | 5.04 | br s | | 13, 15 |
| | | | 5.69 | br s | | |
| 18 | 29.26 | CH3 | 1.39 | s | (3H) | 3, 4, 5, 19 |
| 19 | 175.69 | C | — | — | | — |
| 20 | 16.75 | CH3 | 1.12 | s | (3H) | 1, 5, 9, 10 |

Sugar moiety
Sugar A: β-D-Glucopyranoside

| | | | | | | |
|---|---|---|---|---|---|---|
| 1^{a} | 93.32 | CH | 6.19 | d | 7.8 | 19, 5^{a} |
| 2^{a} | 80.57 | CH | 4.29 | m | | |
| 3^{a} | 78.03 | CH | 4.22 | m | | |
| 4^{a} | 70.58 | CH | 4.21 | m | | |
| 5^{a} | 77.69 | CH | 3.96 | m | | |
| 6^{a} | 69.70 | CH2 | 4.26 | m | | 5^{a}, 1^{e} |
| | | | 4.70 | m | | |

Sugar B: β-D-Glucopyranoside

| | | | | | | |
|---|---|---|---|---|---|---|
| 1^{b} | 105.45 | CH | 5.41 | d | 7.7 | 2^{a} |
| 2^{b} | 76.17 | CH | 4.01 | m | | |
| 3^{b} | 78.17 | CH | 4.20 | m | | |
| 4^{b} | 71.90 | CH | 4.21 | m | | |
| 5^{b} | 78.53 | CH | 3.95 | m | | |
| 6^{b} | 62.96 | CH2 | 4.42 | m | | |
| | | | 4.55 | m | | |

Sugar C: β-D-Glucopyranoside

| | | | | | | |
|---|---|---|---|---|---|---|
| 1^{c} | 106.59 | CH | 5.27 | d | 7.8 | 2^{d} |
| 2^{c} | 77.19 | CH | 4.13 | m | | |
| 3^{c} | 77.91 | CH | 4.23 | m | | |
| 4^{c} | 71.47 | CH | 4.34 | m | | |
| 5^{c} | 78.60 | CH | 3.92 | m | | |
| 6^{c} | 62.55 | CH2 | 4.44 | m | | |
| | | | 4.51 | m | | |

Sugar D: β-D-Glucopyranoside

| | | | | | | |
|---|---|---|---|---|---|---|
| 1^{d} | 97.87 | CH | 5.20 | d | 7.7 | 13 |
| 2^{d} | 84.43 | CH | 4.17 | m | | |
| 3^{d} | 77.88 | CH | 4.43 | m | | |
| 4^{d} | 71.30 | CH | 4.23 | m | | |
| 5^{d} | 77.39 | CH | 3.83 | m | | |
| 6^{d} | 62.27 | CH2 | 4.27 | m | | |
| | | | 4.33 | m | | |

Sugar E: β-D-Glucopyranoside

| | | | | | | |
|---|---|---|---|---|---|---|
| 1^{e} | 105.34 | CH | 4.95 | d | 7.8 | 6^{a} |
| 2^{e} | 75.15 | CH | 3.98 | m | | |
| 3^{e} | 78.29 | CH | 4.14 | m | | |
| 4^{e} | 71.52 | CH | 4.13 | m | | |
| 5^{e} | 78.33 | CH | 3.85 | m | | |
| 6^{e} | 62.67 | CH2 | 4.30 | m | | |
| | | | 4.46 | m | | |

Correlation of all NMR results indicates rebaudioside D9 with five β-D-glucoses attached to steviol aglycone, as depicted with the following chemical structure:

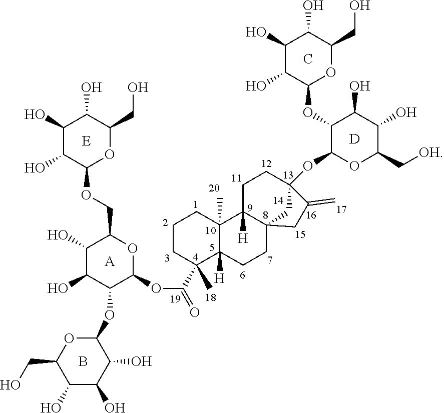

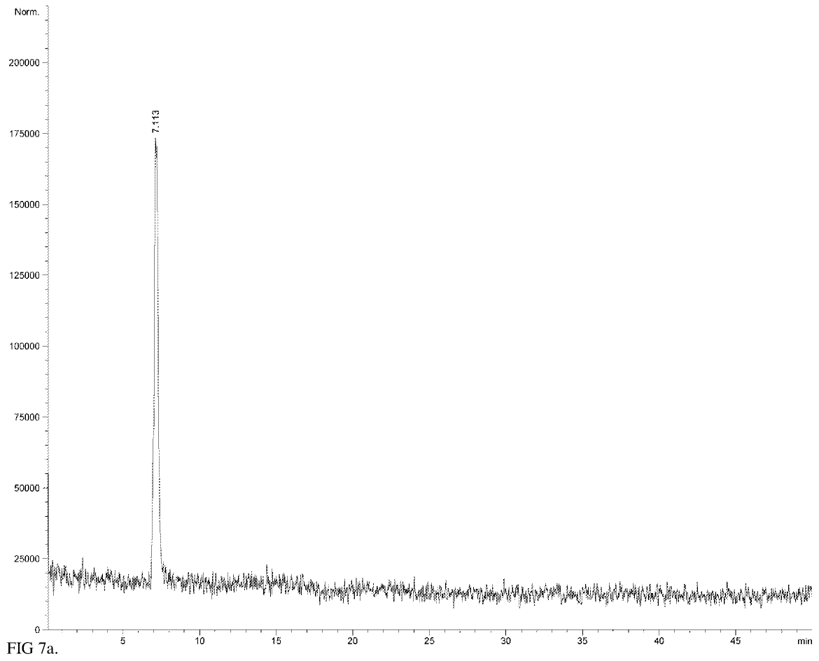

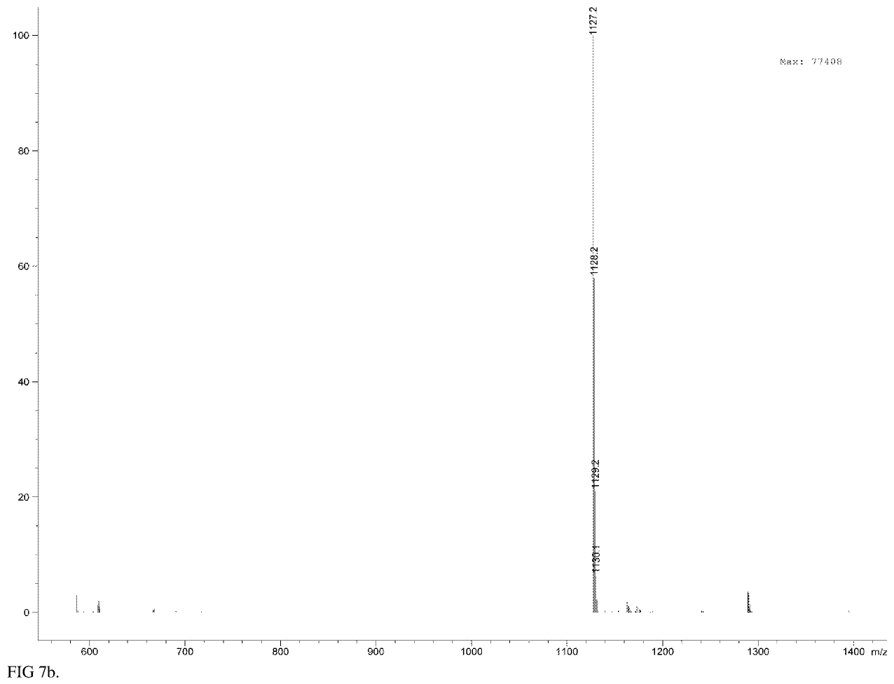

LCMS (FIG. 7a and FIG. 7b) analysis of rebaudioside D9 showed a [M−H]⁻ ion at m/z 1127.2, in good agreement with the expected molecular formula of C₅₀H₈₀O₂₈ (calculated for [C₅₀H₇₉O₂₈]⁻ monoisotopic ion: 1127.5). The MS data confirms that rebaudioside D9 has a molecular formula of C₅₀H₈₀O₂₈. LCMS analysis was performed in the following conditions listed in Table 4.

TABLE 4
Conditions for LCMS analysis

| Column | Agilent Poroshell 120 SB-C18, 4.6 mm × 150 mm, | | |
|---|---|---|---|
| | 2.7 μm | | |
| Temperature | 40° C. | | |
| Mobile Phase | A: Mobile Phase Premix Solution | | |
| | 25% Acetonitrile: 75% Formic Acid (0.1% in Water) | | |
| | B: Mobile Phase Premix Solution | | |
| | 32% Acetonitrile: 68% Formic Acid (0.1% in Water) | | |
| Gradient | Time (min) | A (%) | B (%) |
| | 0 | 100 | 0 |
| | 12.0 | 100 | 0 |
| | 12.5 | 50 | 50 |
| | 13.0 | 0 | 100 |
| | 60.0 | 0 | 100 |

| | |
|---|---|
| Flow rate | 0.5 mL/min |
| Injection | 2 μL |
| Run time | 45 mins |
| Post time | 5 mins |
| Autosampler | Ambient |
| temperature | |
| Detection | DAD and MSD at Negative Scan mode |
| DAD Settings | UV at 210 nm (4 nm bandwidth), Reference: 360 nm |
| | (100 nm bandwidth) |
| MSD Setting | Mode: ES-API, Negative Polarity |
| | Drying gas flow: 13.0 L/min |
| | Nebulizer Pressure: 30 psig |
| | Drying gas temperature: 270° C. |
| | Fragmentor: 50 V |
| | Scan ranges: 500 to 1500 of mass |
| Sample | 1 mg/ml (30% ACN in water) |
| Preparation | |

### Example 9

### Structure Elucidation of Rebaudioside M4

NMR experiments were performed on a Bruker 800 MHz spectrometer, with the rebaudioside M4 sample, prepared according to EXAMPLE 7, dissolved in pyridine-d5. Along with signals from the sample, signals from pyridine-d5 at δ_{C} 123.5, 135.5, 149.9 ppm and δ_{H} 7.19, 7.55, 8.71 ppm were observed. 1H-NMR and 13C-NMR spectra of rebaudioside M4 confirmed the excellent quality of the sample. HSQC shows the presence of an exo-methylene group in the sugar region with a long-range coupling to C-15, observable in the H,H—COSY. Correlation of the signals in the HSQC, HMBC and H,H—COSY reveal the presence of steviol glycoside with the following aglycone structure:

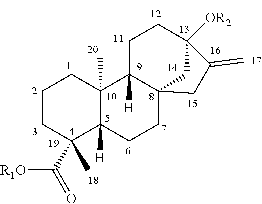

Correlation of HSQC and HMBC shows the presence six anomeric signals, marked as 1a, 1b, 1c, 1d, 1e and 1f. The coupling constant of the anomeric protons of about 8 Hz and/or the NOE-correlations of the anomeric protons allow the identification of these six sugars as β-D-glucopyranosides.

Combined data from HSQC and HMBC reveal the sugar-sugar linkages and sugar-aglycone linkages. The assignment of the sugar sequence was further supported by TOCSY, NOESY and H2BC.

Altogether, results from NMR experiments above were used to assign the chemical shifts of the protons and carbons of the structure of rebaudioside M4 (see Table 5).

TABLE 5
Chemical shifts of rebaudioside M4

| | | | | | |
|---|---|---|---|---|---|
| | δ_{C} | δ_{H} | | HMBC | H2BC |
| Position | [ppm] | [ppm] | Int | (H→C) | (H→C) |

Aglycone moiety

| | | | | | | | |
|---|---|---|---|---|---|---|---|
| 1 | 40.45 | CH2 | 1.62 | m | | 3 | |
| | | | 0.67 | m | | | 2 |
| 2 | 19.93 | CH2 | 2.07 | m | | | 1, 3 |
| | | | 1.37 | m | | | |
| 3 | 37.46 or | CH2 | 2.78 | m | | 1, 5 | |
| | 37.52 | | 1.04 | m | | 4 | 2 |
| 4 | 44.28 | C | — | — | | | |
| 5 | 57.31 | CH | 0.94 | m | | 4, 6, 7, 10, | 6 |
| | | | | | | 18, 19, 20 | |
| 6 | 22.16 | CH2 | 2.11 | m | | | 5, 7 |
| | | | 1.9 | m | | | |
| 7 | 41.62 | CH2 | 1.36 | m | | | |
| | | | 1.25 | m | | | 6 |
| 8 | 42.36 | C | — | — | | | |
| 9 | 53.8 | CH | 0.81 | m | | 8, 10 | |
| 10 | 39.6 | C | — | — | | | |
| 11 | 20.45 | CH2 | 1.58 | m | (2H) | | 9, 12 |
| 12 | 37.46 or | CH2 | 2.03 | m | | 11, 13 | |
| | 37.52 | | 1.88 | m | | | |
| 13 | 86.24 | C | — | — | | | |
| 14 | 44.02 | CH2 | 2.39 | m | | 13, 15, 16 | |
| | | | 1.68 | m | | 8, 9, 12, 13 | |
| 15 | 47.66 | CH2 | 1.99 | m | (2H) | 7, 8, 9, 13, | |
| | | | | | | 16 | |
| 16 | 154.01 | C | — | — | | | |
| 17 | 104.8 | CH2 | 5.61 | br s | | 13, 15 | |
| | | | 4.98 | br s | | 13, 15 | |
| 18 | 28.93 | CH3 | 1.39 | s | (3H) | 3, 4, 5, 19 | |
| 19 | 175.76 | C | — | — | | | |
| 20 | 16.62 | CH3 | 0.99 | s | (3H) | 1, 5, 9, 10 | |

| | | | | | | | | |
|---|---|---|---|---|---|---|---|---|
| | | | J | HMBC | H2BC | | | |
| Position | δ_{C} [ppm] | δ_{H} [ppm] | [Hz] | (H→C) | (H→C) | NOESY | COSY | TOCSY |

Sugar A: β-D-Glucopyranoside

| | | | | | | | | | | |
|---|---|---|---|---|---|---|---|---|---|---|
| 1a | 93.25 | CH | 6.23 | d | 7.6 | 19, 5a | 2a | | 2a | |
| 2a | 77.22 | CH | 4.48 | m | | 1b | 1a, 3a | | | 5a |
| 3a | 88.67 | CH | 4.3 | m | | 2a, 1c | 4a | 5a | | |
| 4a | 69.3 | CH | 4.14 | m | | 6a | 3a | | | |
| 5a | 78.79 | CH | 3.84 | m | | | 4a, 6a | | | |
| 6a | 61.76 | CH2 | 4.36 | m | | | | | | |
| | | | ~4.22 | m | | | | | | |

Sugar B: β-D-Glucopyranoside

| | | | | | | | | | | |
|---|---|---|---|---|---|---|---|---|---|---|
| 1b | 103.69 | CH | 5.78 | d | 7.4 | 2a | 2b | 5b | 2b | |
| 2b | 75.9 | CH | 3.99 | m | | | 1b | | | |
| 3b | 71.2 or | CH | 4.29 | m | | 2b, 4b | | | | |
| | 77.7-78.7 | | | | | | | | | |
| 4b | 72.78 | CH | 4.10 | m | | 6b | | | | |
| 5b | 71.2 or | CH | 3.96 | m | | | 4b, 6b | | | |
| | 77.7-78.7 | | | | | | | | | |
| 6b | 63.52 | CH2 | 4.57 | m | | | | | 5b | 4b, 5b |
| | | | 4.34 | m | | | | | 5b | 4b, 5b |

Sugar C: β-D-Glucopyranoside

| | | | | | | | | | |
|---|---|---|---|---|---|---|---|---|---|
| 1c | 104.55 | CH | 5.32 | d | 7.7 | 3a | 2c | 2c | |
| 2c | 75.4 | CH | 4.01 | m | | | 1c | | |
| 3c | 71.2 or | CH | ~4.20 | m | | | | | |
| | 77.7-78.7 | | | | | | | | |
| 4c | 71.51 | CH | 4.13 | m | | 6c | | | |
| 5c | 71.2 or | CH | 4.02 | m | | | 4c, 6c | | |
| | 77.7-78.7 | | | | | | | | |
| 6c | 62.24 | CH2 | 4.50 | m | | | | | 5c |
| | | | ~4.22 | m | | | | | |

Sugar D: β-D-Glucopyranoside

| | | | | | | | | | |
|---|---|---|---|---|---|---|---|---|---|
| 1d | 106.12 | CH | 5.26 | d | 7.3 | 2f | 2d | 2d | |
| 2d | 77.08 | CH | 4.078 | m | | 1d, 3d | 1d | | |
| 3d | 71.2 or | CH | ~4.20 | m | | | | | |
| | 77.7-78.7 | | | | | | | | |
| 4d | 71.67 | CH | 4.29 | m | | 3d, 6d | 3d | | |
| 5d | 71.2 or | CH | 3.91 | m | | | 4d, 6d | | |
| | 77.7-78.7 | | | | | | | | |
| 6d | 62.77 | CH2 | 4.50 | m | | | | 5d | 5d |
| | | | 4.41 | m | | | | 5d | 5d |

Sugar E: β-D-Glucopyranoside

| | | | | | | | | | |
|---|---|---|---|---|---|---|---|---|---|
| 1e | 104.8 | CH | 5.09 | H2O | 4f | 2e | 5e | 2e | |
| | | | | overlap | | | | | |
| 2e | 74.82 | CH | 4.03 | m | 1e | 1e | | | |
| 3e | 71.2 or | CH | ~4.20 | m | | | | | |
| | 77.7-78.7 | | | | | | | | |
| 4e | 71.35 | CH | ~4.20 | m | | | | | |
| 5e | 71.2 or | CH | 4.02 | m | | 4e, 6e | 1e | | |
| | 77.7-78.7 | | | | | | | | |
| 6e | 62.2 | CH2 | 4.47 | m | | | | 5e | 5e |
| | | | 4.27 | m | | | | | |

Sugar F: β-D-Glucopyranoside

| | | | | | | | | | |
|---|---|---|---|---|---|---|---|---|---|
| 1f | 97.54 | CH | 4.99 | H2O | 13 | 2f | 3f, 5f | 2f | 5f |
| | | | | overlap | | | | | |
| 2f | 82.87 | CH | 4.13 | m | 1d | 1f, 3f | | | |
| 3f | 76.18 | CH | 4.3 | m | | 2f, 4f | 1f, 5f | | |
| 4f | 80.33 | CH | ~4.22 | m | 1e, 3f, | | | | |
| | | | | | 6f | | | | |
| 5f | 75.77 | CH | 3.53 | m | | 6f, 4f | 1f, 3f | | |
| 6f | 61.57 | CH2 | 4.38 | m | | | | | |
| | | | ~4.22 | m | | | | | |

Correlation of all NMR results indicates rebaudioside M4 with six β-D-glucoses attached to steviol aglycone, as depicted with the following chemical structure:

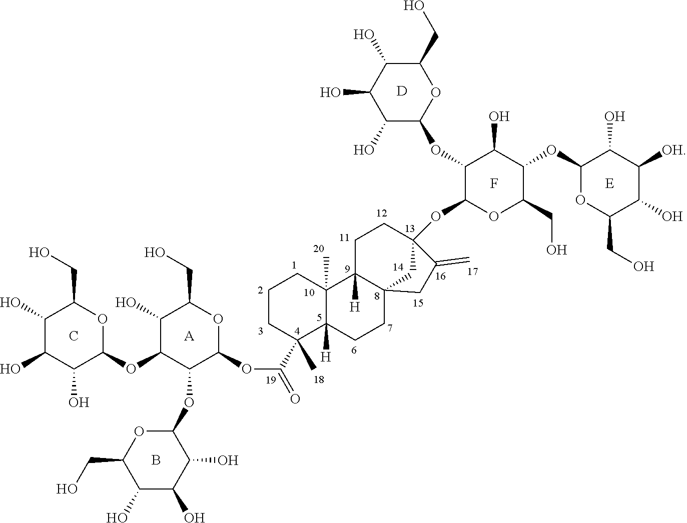

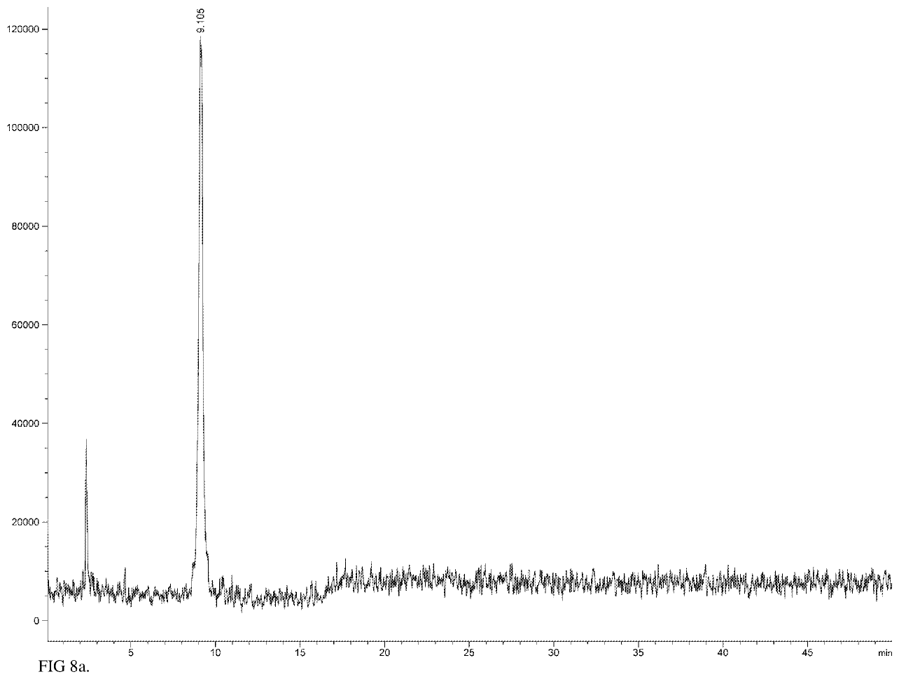

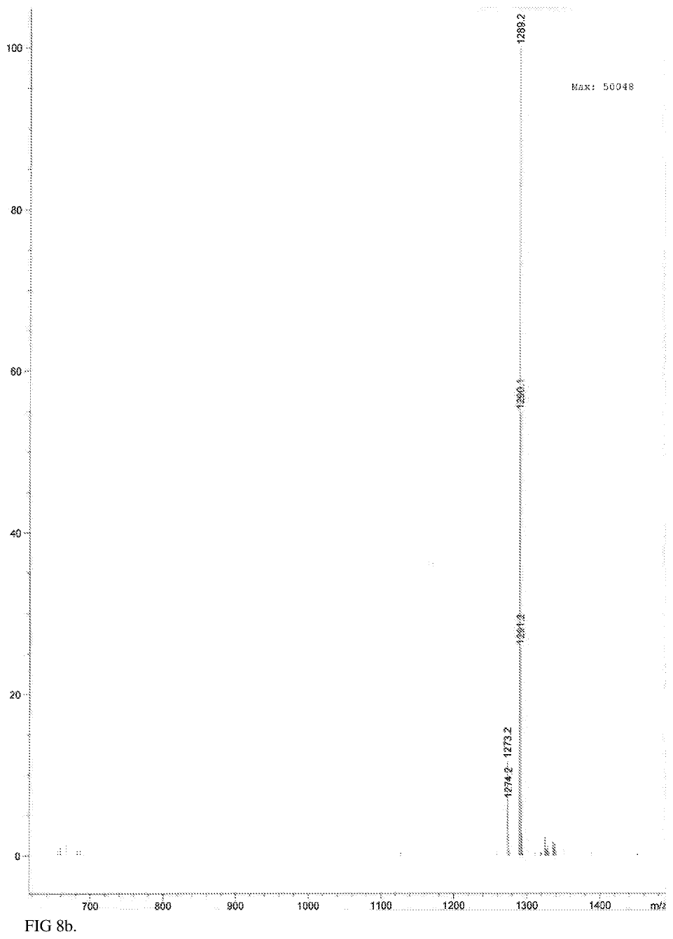

LCMS (FIG. 8a and FIG. 8b) analysis of rebaudioside M4 showed a [M−H]⁻ ion at m/z 1289.2, in good agreement with the expected molecular formula of C56H90033 (calculated for [C₅₆H₈₉O₃₃]⁻ monoisotopic ion: 1289.5). The MS data confirms that rebaudioside M4 has a molecular formula of C₅₆H₉₀O₃₃. LCMS analysis was performed in the conditions listed in Table 4.

We claim:

1. Rebaudioside D9 comprising the formula:

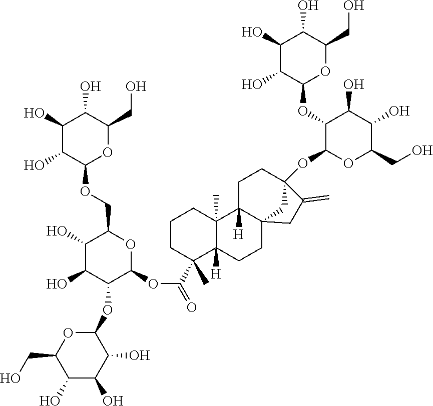

2. A method for producing rebaudioside D9 of claim 1, comprising the steps of:
a. providing a starting composition comprising an organic compound with at least one carbon atom;
b. providing an enzyme preparation or microorganism containing at least one enzyme selected from the group consisting of steviol biosynthesis enzymes, and NDP-glucosyltransferases and optionally NDP-glucose recycling enzymes;
c. contacting the enzyme preparation or microorganism with a medium containing the starting composition to produce a medium comprising rebaudioside D9.

3. A method for producing rebaudioside D9 of claim 1, comprising the steps of:
a. providing a starting composition comprising an organic compound with at least one carbon atom;
b. providing a biocatalyst comprising at least one enzyme selected from the group consisting of steviol biosynthesis enzymes and NDP-glucosyltransferases and optionally NDP-glucose recycling enzymes; and
c. contacting the biocatalyst with a medium containing the starting composition to produce a medium comprising rebaudioside D9.

4. The method of claim 2 further comprising the step of:
d. separating rebaudioside D9 from the medium to provide a highly purified composition of rebaudioside D9.

5. The method of claim 2, wherein the starting composition is selected from the group consisting of steviol, steviolmonoside, steviolmonoside A, steviolbioside, steviolbioside A, steviolbioside G, rubusoside, stevioside, stevioside A, stevioside K, stevioside L, rebaudioside E, rebaudioside E8, rebaudioside E12, other steviol glycosides, polyols, carbohydrates, and combinations thereof.

6. The method of claim 2, wherein the microorganism is selected from the group consisting of E. coli, Saccharomyces sp., Aspergillus sp., Pichia sp., Bacillus sp., and Yarrowia sp.

7. The method of claim 3, wherein the biocatalyst is an enzyme, or a cell comprising one or more enzymes, capable of converting the starting composition to rebaudioside D9.

8. The method of claim 2, wherein the enzyme is selected from the group consisting of a mevalonate (MVA) pathway enzyme, a 2-C-methyl-D-erythritol-4-phosphate pathway (MEP/DOXP) enzyme, geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase, UGT74G1, UGT85C2, UGTS12, UGT76G1, EUGT11, UGT91D2 or mutant variant thereof having >85% amino-acid sequence identity with SEQ ID 2, SEQ ID 3, SEQ ID 4, SEQ ID 5, SEQ ID 6 and SEQ ID 7, respectively.

9. The method of claim 4, wherein the content of rebaudioside D9 in the highly purified composition of rebaudioside D9 is greater than about 80% by weight on a dried basis.

10. A consumable product comprising rebaudioside D9 of claim 1, wherein the product is selected from the group consisting of food, beverages, pharmaceutical compositions, tobacco products, nutraceutical compositions, oral hygiene compositions, and cosmetic compositions.

11. The consumable product of claim 10, wherein the product is selected from the group consisting of foodstuffs, beverages, pharmaceutical compositions, cosmetics, chewing gums, table top products, cereals, dairy products, toothpastes, other oral cavity compositions, alcoholic beverages such as vodka, wine, beer, liquor, and sake, etc., natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, frozen beef, frozen pork, frozen goat, frozen lamb, frozen mutton, frozen poultry like frozen chicken, frozen duck and frozen turkey, frozen venison, frozen fish, frozen crustaceans like frozen crab and frozen lobster, frozen molluscs like frozen clams, frozen oysters, frozen scallops, and frozen mussels, frozen shrimps, frozen octopus, frozen squid, fresh beef, fresh pork, fresh goat, fresh lamb, fresh mutton, fresh poultry like fresh chicken, fresh duck and fresh turkey, fresh venison, fresh fish, fresh crustaceans like fresh crab and fresh lobster, fresh molluscs like fresh clams, fresh oysters, fresh scallops, and fresh mussels, fresh shrimps, fresh octopus, fresh squid, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, medicinal products, and lipsticks.

12. The consumable product of claim 10, further comprising at least one additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, caffeine, flavorants and flavoring ingredients, flavorings with modifying properties (FMP), astringent compounds, proteins or protein hydrolysates, surfactants, emulsifiers, flavonoids, alcohols, polymers and combinations thereof.

13. The consumable product of claim 10, further comprising at least one functional ingredient selected from the group consisting of saponins, antioxidants, dietary fiber sources, fatty acids, vitamins, glucosamine, minerals, preservatives, hydration agents, probiotics, prebiotics, postbiotics, weight management agents, osteoporosis management agents, phytoestrogens, long chain primary aliphatic saturated alcohols, phytosterols and combinations thereof.

14. The consumable product of claim 10, further comprising a compound selected from the group consisting of dulcoside A, dulcoside B, dulcoside C, dulcoside D, rebaudioside 1a, rebaudioside 1b, rebaudioside 1c, rebaudioside 1d, rebaudioside 1e, rebaudioside 1f rebaudioside 1g, rebaudioside 1h, rebaudioside 1i, rebaudioside 1j, rebaudioside 1k, rebaudioside 1l, rebaudioside 1m, rebaudioside 1n, rebaudioside 1o, rebaudioside 1p, rebaudioside 1q, rebaudioside 1r, rebaudioside 1s, rebaudioside 1t, rebaudioside 2a, rebaudioside 2b, rebaudioside 2c, rebaudioside 2d, rebaudioside 2e, rebaudioside 2f rebaudioside 2g, rebaudioside 2h, rebaudioside 2i, rebaudioside 2j, rebaudioside 2k, rebaudioside 2l, rebaudioside 2m, rebaudioside 2n, rebaudioside 2o, rebaudioside 2p, rebaudioside 2q, rebaudioside 2r, rebaudioside 2s, rebaudioside A, rebaudioside AIG, rebaudioside A2, rebaudioside A3, rebaudioside A4, rebaudioside AM, rebaudioside B, rebaudioside B2, rebaudioside C, rebaudioside C2, rebaudioside C3, rebaudioside C4, rebaudioside C5, rebaudioside C6, rebaudioside C7, rebaudioside D, rebaudioside D2, rebaudioside D3, rebaudioside D4, rebaudioside D5, rebaudioside D6, rebaudioside D7, rebaudioside D8, rebaudioside D9, rebaudioside D10, rebaudioside D11, rebaudioside D12, rebaudioside D13, rebaudioside E, rebaudioside E2, rebaudioside E3, rebaudioside E4, rebaudioside E5, rebaudioside E6, rebaudioside E7, rebaudioside E8, rebaudioside E9, rebaudioside E10, rebaudioside E11, rebaudioside E12, rebaudioside E13, rebaudioside F, rebaudioside F1, rebaudioside F2, rebaudioside F3, rebaudioside G, rebaudioside H, rebaudioside H1, rebaudioside H2, rebaudioside H3, rebaudioside H4, rebaudioside H5, rebaudioside H6, rebaudioside H7, rebaudioside I, rebaudioside I2, rebaudioside I3, rebaudioside IX, rebaudioside IXa, rebaudioside IXb, rebaudioside IXc, rebaudioside IXd, rebaudioside J, rebaudioside K, rebaudioside K2, rebaudioside KA, rebaudioside L, rebaudioside L1, rebaudioside M, rebaudioside M2, rebaudioside M3, rebaudioside M4, rebaudioside M5, rebaudioside N, rebaudioside N2, rebaudioside N3, rebaudioside N4, rebaudioside N5, rebaudioside N6, rebaudioside N7, rebaudioside O, rebaudioside O2, rebaudioside O3, rebaudioside O4, rebaudioside O5, rebaudioside O6, rebaudioside O7, rebaudioside Q, rebaudioside Q2, rebaudioside Q3, rebaudioside R, rebaudioside R1, rebaudioside S, rebaudioside T, rebaudioside T1, rebaudioside U, rebaudioside U2, rebaudioside U3, rebaudioside V, rebaudioside V2, rebaudioside VIII, rebaudioside VIIIa, rebaudioside VIIIb, rebaudioside W, rebaudioside W2, rebaudioside W3, rebaudioside WB1, rebaudioside WB2, rebaudioside Y, rebaudioside Z1, rebaudioside Z2, rubusoside, steviolbioside, steviolbioside A, steviolbioside B, steviolbioside C, steviolbioside D, steviolbioside E, steviolbioside F, steviolbioside G, steviolmonoside, steviolmonoside A, stevioside, stevioside A, stevioside B, stevioside C, stevioside D, stevioside E, stevioside E2, stevioside F, stevioside G, stevioside H, stevioside I, stevioside J, stevioside K, stevioside L, stevioside M, SvG7, NSF-02, Mogroside V, siratose, Luo Han Guo, allulose, D-allose, D-tagatose, erythritol, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, siamenoside, sucralose, potassium acesulfame, aspartame, alitame, saccharin, cyclamate, neotame, dulcin, suosan advantame, gymnemic acid, hodulcin, ziziphin, lactisole, glutamate, aspartic acid, glycine, alanine, threonine, proline, serine, lysine, tryptophan, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols, sugar alcohols, L-sugars, L-sorbose, L-arabinose, trehalose, galactose, rhamnose, various cyclodextrins, cyclic oligosaccharides, various types of maltodextrins, dextran, sucrose, glucose, ribulose, fructose, threose, xylose, lyxose, altrose, mannose, idose, lactose, maltose, invert sugar, isotrehalose, neotrehalose, isomaltulose, erythrose, deoxyribose, gulose, talose, erythrulose, xylulose, cellobiose, amylopectin, glucosamine, mannosamine, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, isomerized liquid sugars such as high fructose corn syrups, coupling sugars, soybean oligosaccharides, D-psicose, D-ribose, L-glucose, L-fucose, D-turanose, D-leucrose, 5-ketofructose and combinations thereof.

15. A method for enhancing the sweetness of a beverage or food product, comprising a sweetener comprising the step of:
a. adding a sweetness enhancer comprising rebaudioside D9 of claim 1 to a beverage or food product comprising a sweetener, wherein rebaudioside D9 is present in a concentration at or below the sweetness recognition threshold.

16. The method of claim 15 further comprising the step of adding a sweetness enhancer comprising rebaudioside D9, wherein rebaudioside D9 is present in an amount from about 0.0001% to about 12% by weight.

17. A method for stabilizing the flavor of a beverage or food product, comprising the step of:
a. adding a flavor stabilizer comprising rebaudioside D9 of claim 1 to a beverage or food product, wherein rebaudioside D9 is present in an amount from about 0.0001% to about 12% by weight.

18. A method for modification of flavor and/or taste profile of a beverage or food product, comprising the step of:
a. adding a flavoring with modifying properties (FMP) comprising rebaudioside D9 of claim 1 to a beverage or food product, wherein rebaudioside D9 is present in an amount from about 0.0001% to about 12% by weight.

19. A method for suppressing foaming of a beverage or food product, comprising the step of:
a. adding a foam suppressor comprising rebaudioside D9 of claim 1 to a beverage or food product, wherein rebaudioside D9 is present in an amount from about 0.0001% to about 12% by weight.

20. A method for enhancing the solubility of insoluble material in a beverage or food product, comprising the step of:
a. adding a solubility enhancing agent comprising rebaudioside D9 of claim 1 to a beverage or food product containing insoluble material, wherein rebaudioside D9 is present in an amount from about 0.0001% to about 12% by weight.
